(19) United States
(12) Patent Application Publication
(10) Pub. No.: US 2022/0195053 A1
(43) Pub. Date: Jun. 23, 2022

(54) MODULATION OF WNT SIGNALING IN GASTROINTESTINAL DISORDERS

(71) Applicant: Surrozen Operating Inc., South San Francisco, CA (US)

(72) Inventors: Yang LI, Mountain View, CA (US); Chenggang LU, Foster City, CA (US); Hélène BARIBAULT, Redwood City, CA (US); Wen-Chen YEH, Belmont, CA (US); Liqin XIE, Elmsford, NY (US); I-Chieh WANG, San Bruno, CA (US); Weixu MENG, Arcadia, CA (US)

(73) Assignee: Surrozen Operating Inc., South San Francisco, CA (US)

(21) Appl. No.: 17/438,303

(22) PCT Filed: Mar. 11, 2020
(86) PCT No.: PCT/US2020/022183

§ 371 Date: Sep. 10, 2021

Related U.S. Application Data
(60) Provisional application No. 62/888,749, filed on Aug. 19, 2019.
(60) Provisional application No. 62/816,720, filed on Mar. 11, 2019.

(51) Int. Cl. C07K 16/28; A61P 1/00

(52) U.S. Cl. CPC C07K 16/2863; A61K 2039/505; A61P 1/00

(57) ABSTRACT

The present invention provides methods of treating gastrointestinal disorders with modulators of the WNT signaling pathway. Also provided are methods of dosing and pharmaceutical compositions.

### CROSS REFERENCE TO RELATED APPLICATIONS

This application claims priority to U.S. Provisional Application No. 62/816,729, filed Mar. 11, 2019, and U.S. Provisional Application No. 62/888,749, filed Aug. 19, 2019, each of which is incorporated by reference herein in its entirety.

### FIELD OF THE INVENTION

The present invention provides WNT signal modulators as a treatment for gastrointestinal disorders, in particular, inflammatory bowel diseases.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is SRZN_014_02WO_ST25.txt. The text file is about 102 KB, created on Mar. 11, 2020, and is being submitted electronically via EFS-Web.

### BACKGROUND OF THE INVENTION

The adult intestinal epithelium is characterized by continuous replacement of epithelial cells through a stereo-typed cycle of cell division, differentiation, migration and exfoliation occurring during a 5-7 day crypt-villus transit time. The putative growth factors regulating proliferation within the adult intestinal stem cell niche have not yet been fully identified, although studies have implicated the cell-intrinsic action of β-catenin/Lef/Tcf signaling within the proliferative crypt compartment.

A number of pathological conditions affect the cells of the intestines. Inflammatory bowel disease (IBD) can involve either or both the small and large bowel. Crohn's disease and ulcerative colitis are the best-known forms of IBD, and both fall into the category of “idiopathic” inflammatory bowel disease because the etiology for them is unknown. “Active” IBD is characterized by acute inflammation. “Chronic” IBD is characterized by architectural changes of crypt distortion and scarring. Crypt abscesses can occur in many forms of IBD.

Ulcerative colitis (UC) involves the colon as a diffuse mucosal disease with distal predominance. The rectum is virtually always involved, and additional portions of colon may be involved extending proximally from the rectum in a continuous pattern. The etiology for UC is unknown. Patients with prolonged UC are at increased risk for developing colon cancer. Patients with UC are also at risk for development of liver diseases including sclerosing cholangitis and bile duct carcinoma.

Crohn's disease can involve any part of the GI tract, but most frequently involves the distal small bowel and colon. Inflammation is typically transmural and can produce anything from a small ulcer over a lymphoid follicle (aphthoid ulcer) to a deep fissuring ulcer to transmural scarring and chronic inflammation. One third of cases have granulomas, and extracolonic sites such as lymph nodes, liver, and joints may also have granulomas. The transmural inflammation leads to the development of fistulas between loops of bowel and other structures. Inflammation is typically segmental with uninvolved bowel separating areas of involved bowel. The etiology is unknown, though infectious and immunologic mechanisms have been proposed.

WNT proteins form a family of highly conserved secreted signaling molecules that regulate cell-to-cell interactions during embryogenesis. WNT genes and WNT signaling are also implicated in cancer. Insights into the mechanisms of WNT action have emerged from several systems: genetics in Drosophila and Caenorhabditis elegans; biochemistry in cell culture and ectopic gene expression in Xenopus embryos. Many WNT genes in the mouse have been mutated, leading to very specific developmental defects. As currently understood, WNT proteins bind to receptors of the Frizzled family on the cell surface. Through several cytoplasmic relay components, the signal is transduced to beta-catenin, which then enters the nucleus and forms a complex with TCF to activate transcription of WNT target genes. Expression of WNT proteins varies, but is often associated with developmental process, for example in embryonic and fetal tissues.

The exploration of physiologic functions of WNT proteins in adult organisms has been hampered by functional redundancy and the necessity for conditional inactivation strategies. Dickkopf-1 (Dkk1) has been recently identified as the founding member of a family of secreted proteins that potently antagonize WNT signaling (see Glinka et al. (1998) Nature 391:357-62; Fedi et al. (1999) J Biol Chem 274:19465-72; and Bafico et al. (2001) Nat Cell Biol 3:683-6). Dkk1 associates with both the WNT co-receptors LRPS/6 and the transmembrane protein Kremen, with the resultant ternary complex engendering rapid LRP6 internalization and impairment of WNT signaling through the absence of functional Frizzled/LRP6 WNT receptor complexes Mao et al. (2001) Nature 411:321-5; Semenov et al. (2001) Curr Biol 11:951-61; and Mao et al. (2002) Nature 417:664-7).

Transgenic mice that have a knock-out of the Tcf locus show a loss of proliferative stem cell compartments in the small intestine during late embryogenesis. However, the knockout is lethal, and so has not been studied in adults. In chimeric transgenic mice that allow analysis of adults, expression of constitutively active NH2-truncated β-catenin stimulated proliferation in small intestine crypts, although either NH2-truncated β-catenin or Lef-1/β-catenin fusions induced increased crypt apoptosis as well. Because diverse factors regulate β-catenin/Lef/Tcf-dependent transcription, including non-Frizzled GPCRs and PTEN/PI-3-kinase, the cause of intestinal stem cell defect is not known. Developing pharmacologic agents for the regulation of intestinal epithelium growth is of great interest for clinical purposes.

Exploration of WNT agonists has been hampered by the fact that they are not naturally soluble, diffusible molecules. The present invention provides methods to specifically modulate WNT signaling through particular FZD receptors with engineered soluble WNT agonists to achieve differential effect of epithelial regeneration.

### SUMMARY OF THE INVENTION

The present invention is based, in part, upon the use of WNT agonists to regulate gastrointestinal epithelium proliferation, in particular, in inflammatory bowel diseases.

In one embodiment, the present invention provides a method of treating a subject suffering from a gastrointestinal disorder comprising administering to the subject, an engineered WNT signaling modulator. In certain embodiments, the WNT signaling modulator is an engineered WNT agonist. In further embodiments, the engineered WNT agonist is selected from the group consisting of an engineered polypeptide, an engineered antibody containing at least one epitope binding domain, a small molecule, an siRNA, and an antisense nucleic acid molecule. In another embodiment, the engineered WNT agonist comprises binding compositions that bind to one or more FZD receptors (FZD1-10) and binding compositions that bind to one or more LRP (LRPS-6) receptors. In yet a further embodiment, the binding compositions of the engineered WNT agonist comprise: one or more binding compositions that bind to FZD5, FZD8, FZD1, FZD2, FZD7, FZD5,8, FZD1, 2, 7 or FZD1, 2, 7, 5, 8; FZD4; FZD9; or FZD10; and one or more binding compositions that bind to LRPS, LRP6, or LRPS and 6. In a further embodiment, the engineered WNT agonist comprises one or more binding compositions that bind to FZD5 and/or FZD8; and one or more binding compositions that bind to LRP5 and/or LRP6. In still a further embodiment the engineered WNT agonist comprises a binding composition that binds to FZD5 and FZD8, and a binding composition that binds LRP6. In further embodiments, the WNT agonist has a variable heavy chain sequence of SEQ ID NO: 1, 3, 5, 7, 9, 11, or 13; and a variable light chain sequence of SEQ ID NO: 2, 4, 6, 8, 10, 12, or 14. In another embodiment, the engineered WNT agonist reduces inflammatory cytokine expression in the intestine or colon and/or repairs intestinal epithelium. In some embodiments, the engineered WNT agonist comprises a tissue targeting molecule. In a further embodiment, the tissue targeting molecule is an antibody or fragment thereof that binds to a tissue specific cell surface antigen. In some embodiments, the tissue targeting molecule is selected from the group consisting of Cell surface A33 antigen (GPA33; representative sequence is NCBI polypeptide reference sequence NP_005805.1), Cadherin-17 (CDH17; representative sequence is NCBI polypeptide reference sequence NP_004054.3), and Mucin 13 (cell surface associated (Muc-13; representative sequence is NCBI polypeptide reference sequence NP_149038.3), or a functional fragment or variant thereof. In certain embodiments, the WNT agonist is administered with a binding composition that specifically binds an inflammatory molecule. In further embodiments, the binding composition specifically binding the inflammatory molecule is an antagonist of the inflammatory molecule. In a further embodiment, the antagonist of the inflammatory molecule is an antagonist of TNFα, IL-12, IL-12 and IL-23, or IL-23. In certain embodiments, the gastrointestinal disease is inflammatory bowel disease. In further embodiments, the inflammatory bowel disease is selected from the group consisting of: Crohn's disease (CD), CD with fistula formation, and ulcerative colitis (UC).

The present invention also provides a method of treating a subject suffering from a gastrointestinal disorder comprising administering to the subject, a tissue-specific WNT signal enhancing molecule. In certain embodiments, the WNT signal enhancing molecule comprises: a) a first domain that binds to one or more E3 ubiquitin ligases; and b) a second domain that binds to a tissue specific receptor. In a further embodiment, the E3 ubiquitin ligases are selected from the group consisting of Zinc and Ring Finger Protein 3 (ZNRF3) and Ring Finger Protein 43 (RNF43). In another embodiment, the first domain comprises an R-spondin (RSPO) polypeptide. In a further embodiment, the RSPO polypeptide is selected from the group consisting of RSPO-1, RSPO-2, RSPO-3, and RSPO-4. In certain embodiments, the RSPO polypeptide comprises a first furin domain and a second furin domain. In certain embodiments, the second furin domain is wild-type or is mutated to have lower binding to Leucine-rich repeat-containing G protein coupled receptors 4-6 (LGR4-6). In certain embodiments, the engineered agonist or Wnt signal enhancing molecule incorporates a tissue targeting molecule. In further embodiments, the tissue targeting molecule is an antibody or fragment thereof that binds to a tissue specific cell surface antigen. In certain embodiments, the tissue targeting molecule is selected from the group consisting of GPA33, CDH17, and MUC-13, or a functional fragment or variant thereof. In some embodiments, the WNT agonist is administered with a binding composition that specifically binds an inflammatory molecule. In certain embodiments, the binding composition specific for the inflammatory molecule is an antagonist of the inflammatory molecule. In further embodiments, the antagonist of the inflammatory molecule is an antagonist of TNFα, IL-12, IL-12 and IL-23, or IL-23. In some embodiments, the gastrointestinal disease is inflammatory bowel disease. In further embodiments, the inflammatory bowel disease is selected from the group consisting of: Crohn's disease (CD), CD with fistula formation, and ulcerative colitis (UC).

In another embodiment, the present invention provides for a method of treating a subject suffering from a gastrointestinal disorder comprising administering to the subject, an engineered WNT agonist and an engineered tissue specific WNT signal enhancing molecule. The engineered WNT agonist and the engineered tissue specific WNT signal enhancing molecule may be administered at the same time or at different times. In some embodiments, the subject comprises an effective amount of both during an overlapping time period. In certain embodiments, the engineered WNT agonist comprises one or more binding compositions that bind to FZD5, FZD8, FZD1, FZD2, FZD7, FZD Sand 8, or FZD1, 2, and 7, and one or more binding compositions that bind to LRP5, LRP6, or LRP5, In some embodiments, the engineered WNT agonist comprises a tissue targeting molecule. In certain embodiments, the tissue targeting molecule is an antibody or fragment thereof that binds to a tissue specific cell surface antigen. In further embodiments, the tissue targeting molecule is selected from the group consisting of GPA33, CDH17, and MUC-13, or a functional fragment or variant thereof. In certain embodiments, the engineered WNT signal enhancing molecule comprises a first domain that binds to one or more E3 ubiquitin ligases, and a second domain that binds to a tissue specific receptor. In further embodiments, the E3 ubiquitin ligases are selected from the group consisting of Zinc and Ring Finger Protein 3 (ZNRF3) and Ring Finger Protein 43 (RNF43). In some embodiments, the first domain comprises an R-spondin (RSPO) polypeptide. In other embodiments, the RSPO polypeptide is selected from the group consisting of RSPO-1, RSPO-2, RSPO-3, and RSPO-4. In a further embodiment, the RSPO polypeptide comprises a first furin domain and a second furin domain. In yet a further embodiment, the second furin domain is wild-type or is mutated to have lower binding to Leucine-rich repeat-containing G protein coupled receptors 4-6 (LGR4-6). In further embodiments, the WNT signal enhancing molecule has a heavy chain sequence of SEQ ID NO: 17, 20, or 23; and a light chain sequence of SEQ ID NO: 16, 19, or 22. In some embodiments, the engineered WNT agonist and the engineered tissue specific WNT signal enhancing molecule are administered with a binding composition that specifically binds an inflammatory molecule. In further embodiments, the binding composition specific for the inflammatory molecule is an antagonist of the inflammatory molecule. In yet further embodiments, the antagonist of the inflammatory molecule is an antagonist of TNFα, IL-12, IL-12 and IL-23, or IL-23. In certain embodiments, the gastrointestinal disease is inflammatory bowel disease. In further embodiments, the inflammatory bowel disease is selected from the group consisting of: Crohn's disease (CD), CD with fistula formation, and ulcerative colitis (UC).

In another embodiment, the present invention provides for a method of treating a subject suffering from a gastrointestinal disorder comprising administering to the subject, an engineered WNT agonist and an engineered tissue specific WNT signal enhancing combination molecule. In certain embodiments, the combination molecule comprises: a) the engineered WNT agonist comprising one or more binding compositions that bind to FZD5, FZD8, FZD1, FZD2, FZD7, FZD Sand 8, or FZD1, 2, and 7, and one or more binding compositions that bind to LRP5, LRP6, or LRP5 and b) the engineered WNT signal enhancing molecule comprising a first domain that binds to one or more E3 ubiquitin ligases, and a second domain that binds to a tissue specific receptor. In further embodiments, the E3 ubiquitin ligases are selected from the group consisting of Zinc and Ring Finger Protein 3 (ZNRF3) and Ring Finger Protein 43 (RNF43). In some embodiments, the first domain comprises an R-spondin (RSPO) polypeptide. In other embodiments, the RSPO polypeptide is selected from the group consisting of RSPO-1, RSPO-2, RSPO-3, and RSPO-4. In a further embodiment, the RSPO polypeptide comprises a first furin domain and a second furin domain. In yet a further embodiment, the second furin domain is wild-type or is mutated to have lower binding to Leucine-rich repeat-containing G protein coupled receptors 4-6 (LGR4-6). In some embodiments, combination molecule incorporates a tissue targeting molecule. In certain embodiments, the tissue targeting molecule is an antibody or fragment thereof that binds to a tissue specific cell surface antigen. In further embodiments, the tissue targeting molecule is selected from the group consisting of GPA33, CDH17, and MUC-13, or a functional fragment or variant thereof. In further embodiments, the WNT signal enhancing molecule has a heavy chain sequence of SEQ ID NO: 17, 20, or 23; and a light chain sequence of SEQ ID NO: 16, 19, or 22. In some embodiments, the combination molecule is administered with a binding composition that specifically binds an inflammatory molecule. In further embodiments, the binding composition specific for the inflammatory molecule is an antagonist of the inflammatory molecule. In yet further embodiments, the antagonist of the inflammatory molecule is an antagonist of TNFα, IL-12, IL-12 and IL-23, or IL-23. In certain embodiments, the gastrointestinal disease is inflammatory bowel disease. In further embodiments, the inflammatory bowel disease is selected from the group consisting of: Crohn's disease (CD), CD with fistula formation, and ulcerative colitis (UC).

In particular embodiments of any of the methods disclosed herein, the WNT agonist is selected from those disclosed in any of the following: PCT Application Publication No. WO 2016/040895; US Application Publication No. US 2017-0306029; US Application Publication No. US 2017-0349659; PCT Application Publication No. WO 2019/126398; or PCT Application Publication No. WO 2020/01030. In particular embodiments of any of the methods disclosed herein, the tissue-specific WNT signal enhancing molecule is selected from those disclosed in any of the following: PCT Application Publication No. WO 2018/140821; US Application Publication No. US 2020-0048324; or PCT Application Publication No. WO 2020/14271, all of which are herein incorporated by reference in their entireties.

In another embodiment, the disclosure provides a polypeptide that specifically binds Frizzed 5 (FZD5) and Frizzled 8 (FZD8), wherein the polypeptide comprises a sequence having at least 80%, at least 90%, or at least 95% homology to a sequence set forth in any of SEQ ID NOs: 33-40. In some embodiments, the polypeptide comprises an antibody or antibody binding fragment. In some embodiments, the polypeptide comprises at least 5 or all six of the CDRs present in any of the sequences set forth in any one of SEQ ID NOs: 33-40. In some embodiments, said polypeptide comprises six of the CDRs present in any of the sequences set forth in any one of SEQ ID NOs: 33-40, wherein one or more of the CDRs optionally comprises one, two, or three amino acid modifications, optionally a point mutation, an amino acid deletion, or an amino acid insertion.

In a related embodiment, the disclosure provides an engineered WNT agonist comprising: (a) one or more binding domains that bind to FZD5 and FZD8, wherein at least one of the one or more binding domains comprises a polypeptide comprising a sequence having at least 80%, at least 90%, or at least 95% homology to a sequence set forth in any of SEQ ID NOs: 33-40; and (b) one or more binding domains that bind to LRP5, LRP6, or both LRP5 and LRP6. In some embodiments, the engineered WNT agonist comprises a polypeptide sequence having at least 80%, at least 90%, at least 95%, or at least 98% homology to any one of SEQ ID NOs: 7-14. In some embodiments, the engineered WNT agonist comprises: a polypeptide sequence having at least 80%, at least 90%, or at least 95% homology to SEQ ID NO: 7 and a polypeptide sequence having at least 80%, at least 90%, or at least 95% homology to SEQ ID NO:8; a polypeptide sequence having at least 80%, at least 90%, or at least 95% homology to SEQ ID NO: 9 and a polypeptide sequence having at least 80%, at least 90%, or at least 95% homology to SEQ ID NO:10; a polypeptide sequence having at least 80%, at least 90%, or at least 95% homology to SEQ ID NO: 11 and a polypeptide sequence having at least 80%, at least 90%, or at least 95% homology to SEQ ID NO:12; or a polypeptide sequence having at least 80%, at least 90%, or at least 95% homology to SEQ ID NO: 13 and a polypeptide sequence having at least 80%, at least 90%, or at least 95% homology to SEQ ID NO:14.

In another related embodiment, the disclosure provides a combination molecule comprising: a) an engineered WNT agonist disclosed herein; and b) an engineered WNT signal enhancing molecule comprising a first domain that binds to one or more E3 ubiquitin ligases; and a second domain that binds to a tissue specific receptor. In another embodiment, the disclosure provides a pharmaceutical composition comprising a polypeptide, engineered WNT agonist, or combination molecule disclosed herein.

In a related embodiments, the disclosure provides a polypeptide that specifically binds Frizzed 5 (FZD5) and Frizzled 8 (FZD8), wherein the polypeptide comprises one or more sequence having at least 80%, at least 90%, at least 95%, or at least 98% homology to a sequence set forth in any of SEQ ID NOs: 33-40 or encoded by any of SEQ ID NOs: 33-40. In some embodiments, the polypeptide of claim 49, wherein said polypeptide comprises an antibody or antibody binding fragment. In some embodiments, said antibody or antibody binding fragment comprises at least 5 or all six of the CDRs present in any of the following combinations of sequence: SEQ ID NOs:33 and 34; SEQ ID NOs:35 and 36; SEQ ID NOs:37 and 38; or SEQ ID NOs:39 and 40. In some embodiments, said polypeptide comprises six of the CDRs present in any of the of the following combinations of sequence: SEQ ID NOs:33 and 34; SEQ ID NOs:35 and 36; SEQ ID NOs:37 and 38; or SEQ ID NOs:39 and 40, wherein one or more of the CDRs comprises one, two, or three amino acid modifications, optionally a point mutation, an amino acid deletion, or an amino acid insertion. In another embodiment, the disclosure provides an engineered WNT agonist comprising: one or more binding domains that bind to FZD5 and FZD8, wherein at least one of the one or more binding domains comprises a polypeptide that specifically binds Frizzed 5 (FZD5) and Frizzled 8 (FZD8), e.g., any disclosure herein; and one or more binding domains that bind to LRP5, LRP6, or both LRP5 and LRP6. The disclosure also provides a combination molecule comprising: an engineered WNT agonist disclosed herein; and an engineered WNT signal enhancing molecule comprising a first domain that binds to one or more E3 ubiquitin ligases; and a second domain that binds to a tissue specific receptor.

In a related embodiment, the disclosure provides a method of treating a subject suffering from a gastrointestinal disorder comprising administering to the subject an engineered WNT agonist, an engineered WNT signal enhancing molecule, and/or a combination molecule disclosed herein, or a pharmaceutical composition comprising an engineered WNT agonist or combination molecule disclosed herein. In some embodiments, the gastrointestinal disorder is an inflammatory bowel disease, optionally selected from the group consisting of: Crohn's disease (CD), CD with fistula formation, and ulcerative colitis (UC). Any of the methods disclosed herein may be practiced using any of the engineered WNT agonists, engineered WNT signal enhancing molecules, and/or combination molecules disclosed herein.

### BRIEF DESCRIPTION OF THE DRAWINGS

FIGS. 1A-1L shows the expression of Frizzled receptors (FZD) 1, 2, 3, 4, 5, 6, 7, 8, 9 and 10 in mouse small intestine (FIG. 1A-1J), as well as FZD5 and FZD7 in human colon (FIGS. 1K and 1L), as detected by RNAscope® 2.5 HD Assay-Red. The number of red dots in the images indicates FZD receptor expression levels. Enlarged view of selected regions are shown in the insets of FIGS. 1K (FZD5) and 1L (FZD7).

FIG. 2 shows the activity of recombinant, soluble WNT agonists in tissue culture cells. Signaling activities of the WNT agonists were tested by Super TOPFlash luciferase reporter (STF) assay. Dose response curves for R2M3-26, 1RC07-03, and R2M13-03luciferase reporter activities were measured as indicated on the graph.

FIGS. 3A-3E shows the activities of different FZD receptor specific recombinant WNT agonists on mouse intestinal organoids are shown. Mouse small intestinal organoids were treated with R2M3-26 (FIG. 3A), 18R5-DKK1c scFv (FIG. 3B), C) R2M13-03 (FIGS. 3C and D) 1RC07-03 (FIG. 3D), in the presence of 1 μM IWP2 (Porcupine inhibitor) in basal medium. FIG. 3E shows control organoids treated with only 1 μM IWP2. F: normal organoids grown in basal media. Scale bars in FIGS. 3A, 3B, and 3E are at 200 μm and FIGS. 3C and 3D: are at 400 μm.

FIG. 4 shows immunohistochemical staining of a mouse small intestinal organoid after treatment with 100 nM of R2M3-26 are shown stained with anti-Ki67 (red) and anti-E-Cadherin (green) to illustrate cell proliferation upon WNT agonist treatment.

FIG. 5A shows a schematic diagram of experimental protocol used for in vivo studies in a Dextran Sulfate Sodium (DSS) induced acute colitis mouse model. Red arrows indicate daily body weight (BW), fecal score and fecal occult blood tests. Top arrows (days 4 and 7) and bottom arrows (days 4, 5, 6, 7, 8 and 9) above the bar indicate times of treatment twice weekly and daily, respectively. FIGS. 5B and 5C show graphs of body weight and fecal scores over time with treatment of WNT agonists and/or R-Spondin 2 (RSPO2-Fc). For FIG. 5B, the lines from top to bottom at day 9 correspond to: No DSS, RSPO2-hFc/R2M3 26 daily, RSPO2-hFc/R2M3 26 2/wk, R2M3-26 (10 mpk) 2/wk, RSPO2-hFc 3 mpk daily, anti-GFP, and RSPO2-hFc 3 mpk 2/wk. For FIG. 5C, the lines from top to bottom at day 9 correspond to RSPO2-hFc 3 mpk daily, RSPO2-hFc 3 mpk 2/wk, anti-GFP, R2M-26 (10 mpk) 2/wk, RSPO2-hFc/R2M3 26 2/wk, and RSPO2-hFc/R2M3 26 daily. The RSPO2-Fc/R2M3-26 combo treatment, twice weekly or daily, significantly improved DAI at day 9 compared to negative controls. R2M3-26 alone and combo treatments significantly improved body weight at day 10 (*P value, 0.05; **P value <0.01, ***P value <0.001, ****P value <0.0001)

FIGS. 6A-6E show pathology image analysis of colitis models with the treatment of R2M3-26 and RSPO2-Fc, alone and in combination.

FIGS. 7A-7E show semi-quantitative analysis of degree of colitis following treatment of R2M3-26 and RSPO2-Fc, alone and in combination. R2M3-26 treatment significantly decreased the histology scores on mucosa erosion, inflammatory severity, crypt hyperplasia, and goblet cell loss at day 10 (*P value, 0.05; **P value <0.01, ***P value). Histology scoring was assessed as described in, e.g., Geboes, et al. (2000) Gut 47:404-409.

FIG. 8 shows histological images of transverse sections of small intestine. R2M3-26 alone did not cause small intestine hyperplasia, while RSPO2-Fc alone and combination treatment of R2M3-26 and induced hyperplasia.

FIGS. 9A-9J show that R2M3-26, RSPO2-Fc and combination of R2M3-26 and RSPO2-Fc treatments both reduced serum inflammatory cytokine levels of IFN-γ, IL-113, IL-12p70, and TNF-α (*P value, 0.05; **P value <0.01, ***P value <0.001, ****P value <0.0001). For each graph, the bars from left to right are as follows: blue—no DSS treatment; green—aGFP control; purple—R2M3-26 (10 mpk) 2×/week; orange—RSPO2-hFc (3 mpk) 2×/week; black—RSPO2-hFc (3 mpk) daily; brown—RSPO2-hFc (3 mpk)+R2M3-26 (10 mpk) 2×/week; and dark blue—RSPO2-hFc (3 mpk)+R2M3-26 (10 mpk) daily.

FIGS. 10A-10B show body weight loss and fecal score in DSS induced acute colitis model (4% DSS for 7 days followed by 1% DSS until termination). For FIG. 10A, the lines from top to bottom at day 10 correspond to: No DDS, R2M3-26 (10 mpk) 2/wk, R2M13-26 (10 mpk) 2/wk, C07-26 3 mpk 2/wk, RSPO2/R2M3-26 2/wk, DSS PBS, and anti-GFP. For FIG. 10B, the lines from top to bottom at day 10 correspond to: RSPO2/R2M3-26 2/wk, anti-GFP, DSS PBS, R2M3-26 (10 mpk) 2/wk, R2M3-26 (10 mpk) daily, C07-26 3 mpk 2/wk, and R2M13-26 10 mpk 2/wk. Among the DSS treated groups, the R2M3-26, R2M13-26, and 1RC07-26 treatments, twice weekly, significantly improved body weight (FIG. 10A) and fecal score (FIG. 10B) at day 10 compared to negative controls (PBS or aGFP). (*P value, 0.05; **P value <0.01, ***P value <0.001, ****P value <0.0001).

FIGS. 11A-11H show that WNT agonist treatment repaired colon epithelium damage in DSS model. Histological evaluation of the transverse colon of DSS model mice showed colon epithelial damage including inflammation extending from the mucosa to the serosa, crypt hyperplasia, goblet cell loss and ulceration. The R2M3-26, R2M13-26, and 1RC07-26 treatments effectively repaired the colon epithelium, decreasing the epithelial erosion, goblet cell loss and neutrophils migration.

FIGS. 12A-12H show transverse sections of small intestine in DSS colitis model mice untreated, treated with WNT agonists, or a combination of WNT agonists and RSPO2-hFc. R2M3-26, R2M13-26, or 1RC07-3 did not cause small intestine hyperplasia, while the combination treatment of R2M3-26 and RSPO2-Fc induced small intestine hyperplasia.

FIGS. 13A-13F show that WNT agonist treatments reduced inflammatory cytokine levels of TNF-α, IL-6 and IL-8 in the serum and in colon tissue (*P value, 0.05; **P value <0.01, ***P value <0.001, ****P value <0.0001). For each graph, the bars from left to right are as shown below.

FIGS. 14A-14B show that R2M13-26 treatment showed dose dependent efficacy in DAI in the DSS model. R2M13-26 treatment at 0.3, 1, 3, 10 mpk, twice weekly. and treatment at 1, 3, 10, 30 mpk, once weekly, both reduced DAI with a dose response pattern (FIG. 14B) (*P value, 0.05; **P value <0.01, ***P value <0.001, ****P value <0.0001). For FIG. 14A, the lines at the ten day time point correlate from top to bottom with the figure legend from top to bottom. For FIG. 14B, the lines at the ten day time point correlate from top to bottom to: DSS anti-GFP 10 mpk 2/wk, R2M13-26 1 mpk 1/wk, R2M13-26 30 mpk 1/wk, R2M13-26 10 mpk 1/wk, and R2M13-26 3 mpk 1/wk.

FIGS. 15A-15J show histological evaluation of the cross sections of transverse colon of DSS model mice. Colon epithelial damage included neutrophils infiltration, edema, crypt hyperplasia, goblet cell loss and ulceration (FIG. 15B). The R2M13-26 treatments, with different dose and frequency, all showed improved colon histology, repair of the epithelial erosion as well as decreased goblet cell loss and neutrophils migration in the DSS colitis mice.

FIGS. 16A-16C show that R2M13-26 treatments, with different dose and frequency, all reduced inflammatory cytokine levels of TNF-α, IL-6, and IL-8 in the serum (*P value, 0.05; **P value <0.01, ***P value <0.001, ****P value <0.0001).

FIG. 17A-17C show that R2M13-26 treatments, with different dose levels and frequencies, all reduced inflammatory cytokine levels of TNF-α, IL-6, and IL-8 in the colon tissue (*P value, 0.05; **P value <0.01, ***P value <0.001, ****P value <0.0001).

FIG. 18: Activity of four FZD5,8-specific WNT agonists. Signaling activities of the FZD5,8-specific agonists were tested by Super TOPFlash luciferase reporter (STF) assay. Dose response curves for 57SE8-26, 57SB8-26, 174R-E01-26 and 57SA10-26 luciferase reporter activities were measured as indicated and compared to the activity of R2M13-26 in the same assay.

FIGS. 19A-19D show efficacy of four FZD5,8-specific WNT agonists in acute DSS model. FIG. 19A shows that treatment with the four FZD5,8-specific WNT agonists all showed efficacy by lowering the Disease Activity Index (DAI; see Geboes, et al. (2000) Gut 47:404-409) in DSS model. WNT agonist treatment at 10 mpk, twice weekly, significantly reduced DAI (*P value, 0.05; **P value <0.01, ***P value <0.001, ****P value <0.0001) as compared to anti-GFP control. The lines from top to bottom at day 8 correspond to: anti-GFP, 57SE8-26, 57SB8-26, 174RE01-26, R2M13-26, 57SA10-26, and No DSS.

FIGS. 19B-19D show that treatments of four different FZD5,8-specific WNT agonists, comparing to R2M13-26 at the same dose, all reduced inflammatory cytokine levels of TNF-α, IL-6, and IL-8 in the serum (*P value, 0.05; **P value <0.01, ***P value <0.001, ****P value <0.0001).

FIG. 20 shows antibody clone, C14, in an IgG format (see, e.g., WO2016168607A1), binding to human intestine HT29 cell line, which expresses MUC-13. Two additional MUC-13 binders C4 and C7, (see, e.g., WO2016168607A1), which were also expressed as full-length antibodies, failed to exhibit specific binding to the human intestine cell HT29 (FIGS. 20A-20C). Non-specific binding was assessed using the HEK293 cell line (FIGS. 20D-20F) which does not express MUC-13. Cell surface binding of the MUC-13 antibodies was examined by FACS at 10 nM. C14 showed a distinct FACS shift on HT29 cells but not on HEK293 cells, suggesting specific binding.

FIG. 21 shows signaling activities of MUC-13 targeted mutant RSPO2 (mutRSPO2) fusions were tested by Super TOPFlash luciferase reporter (STF) assay in HT29 cells or HEK293 cells. MutRSPO2 has amino acid mutations in the Furin2 binding domain, thus reducing binding to LGR1-4 (see, e.g., WO2020014271). Dose response curves for C4-mutRSPO2, C7-mutRSPO2, and C14-mutRSPO2 luciferase reporter activities were measured as indicated on the graph. C14-mutRSPO demonstrated a specific left shift of the dose response curve only in HT29 cells, with an EC50 comparable to wildtype Fc-RSPO2.

FIG. 22 shows that growth of human small intestine organoids was maintained when wildtype RSPO was replaced with C14-mutRSPO in the media. Human small intestine organoids were grown in basal media in which RSPO-1 was replaced by a non-epithelial cell (e.g., hepatocytes) targeted mutRSPO1 (ASGR1-mutRSPO1; see, e.g. WO2020014271; and WO2018140821) at the concentration dilution series indicated (FIGS. 22A-22C) or by C14-mutRSPO2 at the same concentration (FIGS. 22D-22F). While organoids grown in ASGR1-mutRSPO1 stopped growing and started to degenerate, similar to what observed when growing in basal media without any RSPO (FIG. 22G), C14-mutRSPO was able to maintain organoid growth similar to IntestiCult™ (StemCell Technologies) media which contains wildtype RSPO (FIG. 22H).

### DETAILED DESCRIPTION

As used herein, including the appended claims, the singular forms of words such as “a,” “an,” and “the,” include their corresponding plural references unless the context clearly dictates otherwise.

All references cited herein are incorporated by reference to the same extent as if each individual publication, patent application, or patent, was specifically and individually indicated to be incorporated by reference.

### I. Definitions

“Activity” of a molecule may describe or refer to the binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

The terms “administering” or “introducing” or “providing”, as used herein, refer to delivery of a composition to a cell, to cells, to tissues, to tissue organoids, and/or to organs of a subject, or to a subject. Such administering or introducing may take place in vivo, in vitro or ex vivo.

As used herein, the term “antibody” means an isolated or recombinant binding agent that comprises the necessary variable region sequences to specifically bind an antigenic epitope. Therefore, an antibody is any form of antibody or fragment thereof that exhibits the desired biological activity, e.g., binding the specific target antigen. Thus, it is used in the broadest sense and specifically covers monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to scFv, Fab, and Fab2, so long as they exhibit the desired biological activity.

“Antibody fragments” comprise a portion of an intact antibody, for example, the antigen-binding or variable region of the intact antibody. Examples of antibody fragments include Fab, Fab′, F(ab′)2, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

The term “antigen” refers to a molecule or a portion of a molecule capable of being bound by a selective binding agent, such as an antibody, and 30 additionally capable of being used in an animal to produce antibodies capable of binding to an epitope of that antigen. In certain embodiments, a binding agent (e.g., a WNT surrogate molecule or binding region thereof, or a WNT antagonist) is said to specifically bind an antigen when it preferentially recognizes its target antigen in a complex mixture of proteins and/or macromolecules.

The term “antigen-binding fragment” as used herein refers to a polypeptide fragment that contains at least one CDR of an immunoglobulin heavy and/or light chain, or of a Nanobody® (Nab), that binds to the antigen of interest, in particular to one or more FZD receptors, or to LRP5 and/or LRP6. In this regard, an antigen-binding fragment of the herein described antibodies may comprise 1, 2, 3, 4, 5, or all 6 CDRs of a VH and VL from antibodies that bind one or more FZD receptors or LRP5 and/or LRP6.

As used herein, the terms “biological activity” and “biologically active” refer to the activity attributed to a particular biological element in a cell. For example, the “biological activity” of an WNT agonist, or fragment or variant thereof refers to the ability to mimic or enhance WNT signals. As another example, the biological activity of a polypeptide or functional fragment or variant thereof refers to the ability of the polypeptide or functional fragment or variant thereof to carry out its native functions of, e.g., binding, enzymatic activity, etc. In some embodiments, a functional fragment or variant retains at least 20%, at least 30%, at least 40%, at least 50%, at least 60%, at least 70%, at least 80%, at least 90%, or at least 100% of an activity of the corresponding native protein or nucleic acid. As a third example, the biological activity of a gene regulatory element, e.g. promoter, enhancer, Kozak sequence, and the like, refers to the ability of the regulatory element or functional fragment or variant thereof to regulate, i.e. promote, enhance, or activate the translation of, respectively, the expression of the gene to which it is operably linked.

The term “bifunctional antibody,” as used herein, refers to an antibody that comprises a first arm having a specificity for one antigenic site and a second arm having a specificity for a different antigenic site, i.e., the bifunctional antibodies have a dual specificity.

“Bispecific antibody” is used herein to refer to a full-length antibody that is generated by quadroma technology (see Milstein et al., Nature, 305(5934): 537-540 (1983)), by chemical conjugation of two different monoclonal antibodies (see, Staerz et al., Nature, 314(6012): 628-631 (1985)), or by knob-into-hole or similar approaches, which introduce mutations in the Fc region (see Holliger et al., Proc. Natl. Acad. Sci. USA, 90(14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences), and is monovalent for each antigen to which it binds.

By “comprising,” it is meant that the recited elements are required in, for example, the composition, method, kit, etc., but other elements may be included to form the, for example, composition, method, kit etc. within the scope of the claim. For example, an expression cassette “comprising” a gene encoding a therapeutic polypeptide operably linked to a promoter is an expression cassette that may include other elements in addition to the gene and promoter, e.g. poly-adenylation sequence, enhancer elements, other genes, linker domains, etc.

By “consisting essentially of,” it is meant a limitation of the scope of the, for example, composition, method, kit, etc., described to the specified materials or steps that do not materially affect the basic and novel characteristic(s) of the, for example, composition, method, kit, etc. For example, an expression cassette “consisting essentially of” a gene encoding a therapeutic polypeptide operably linked to a promoter and a polyadenylation sequence may include additional sequences, e.g. linker sequences, so long as they do not materially affect the transcription or translation of the gene. As another example, a variant, or mutant, polypeptide fragment “consisting essentially of” a recited sequence has the amino acid sequence of the recited sequence plus or minus about 10 amino acid residues at the boundaries of the sequence based upon the full length naïve polypeptide from which it was derived, e.g. 10, 9, 8, 7, 6, 5, 4, 3, 2 or 1 residue less than the recited bounding amino acid residue, or 1, 2, 3, 4, 5, 6, 7, 8, 9, or 10 residues more than the recited bounding amino acid residue.

By “consisting of,” it is meant the exclusion from the composition, method, or kit of any element, step, or ingredient not specified in the claim. For example, a polypeptide or polypeptide domain “consisting of” a recited sequence contains only the recited sequence.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

An “epitope” is specific region on an antigen that an antibody recognizes and binds to, and is also referred to as the “antigenic determinant”. An epitope is usually 5-8 amino acids long on the surface of the protein. Proteins are three dimensionally folded structures, and an epitope may only be recognized in its form as it exists in solution, or its native form. When an epitope is made up of amino acids that are brought together by the three-dimensional structure, the epitope is conformational, or discontinuous. If the epitope exists on a single polypeptide chain, it is a continuous, or linear epitope. Depending on the epitope an antibody recognizes, it may bind only fragments or denatured segments of a protein, or it may also be able to bind the native protein.

The portion of an antibody or antibody fragment thereof that recognizes an epitope is referred to as the “epitope binding domain” or “antigen binding domain”. The epitope or antigen binding domain of an antibody or antibody fragment is in the Fab fragment and the effector functions in the Fc fragment. Six segments, known as complementarity determining regions (CDRs) within the variable regions (V_{H} and V_{L}) of the heavy and light chains loop out from the framework (FR regions) globular structure of the rest of the antibody and interact to form an exposed surface at one end of the molecule. This is the antigen binding domain. Generally, 4-6 of the CDRs will be directly involved in binding antigen, although fewer can provide the main binding motifs.

An “expression vector” is a vector, e.g. plasmid, minicircle, viral vector, liposome, and the like as discussed herein or as known in the art, comprising a region which encodes a gene product of interest, and is used for effecting the expression of the gene product in an intended target cell. An expression vector also comprises control elements, e.g. promoters, enhancers, UTRs, miRNA targeting sequences, etc., operatively linked to the encoding region to facilitate expression of the gene product in the target. The combination of control elements and a gene or genes to which they are operably linked for expression is sometimes referred to as an “expression cassette,” a large number of which are known and available in the art or can be readily constructed from components that are available in the art.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (scFv), Nanobodies®, variants thereof, fusion proteins comprising an antigen-binding fragment of a monoclonal antibody, humanized monoclonal antibodies, chimeric monoclonal antibodies, and any other modified configuration of the immunoglobulin molecule that comprises an antigen-binding fragment (epitope recognition site) of the required specificity and the ability to bind to an epitope, including WNT surrogate molecules disclosed herein. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”.

The term “native” or “wild-type” as used herein refers to a nucleotide sequence, e.g. gene, or gene product, e.g. RNA or protein, that is present in a wild-type cell, tissue, organ or organism. The term “variant” as used herein refers to a mutant of a reference polynucleotide or polypeptide sequence, for example a native polynucleotide or polypeptide sequence, i.e. having less than 100% sequence identity with the reference polynucleotide or polypeptide sequence. Put another way, a variant comprises at least one amino acid difference (e.g., amino acid substitution, amino acid insertion, amino acid deletion) relative to a reference polynucleotide sequence, e.g. a native polynucleotide or polypeptide sequence. For example, a variant may be a polynucleotide having a sequence identity of 50% or more, 60% or more, or 70% or more with a full length native polynucleotide sequence, e.g. an identity of 75% or 80% or more, such as 85%, 90%, or 95% or more, for example, 98% or 99% identity with the full length native polynucleotide sequence. As another example, a variant may be a polypeptide having a sequence identity of 70% or more with a full length native polypeptide sequence, e.g. an identity of 75% or 80% or more, such as 85%, 90%, or 95% or more, for example, 98% or 99% identity with the full length native polypeptide sequence. Variants may also include variant fragments of a reference, e.g. native, sequence sharing a sequence identity of 70% or more with a fragment of the reference, e.g. native, sequence, e.g. an identity of 75% or 80% or more, such as 85%, 90%, or 95% or more, for example, 98% or 99% identity with the native sequence.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the worldwide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970)

Of interest is the BestFit program using the local homology algorithm of Smith and Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

A “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

“Recombinant,” as applied to a polynucleotide means that the polynucleotide is the product of various combinations of cloning, restriction or ligation steps, and other procedures that result in a construct that is distinct from a polynucleotide found in nature.

“RNA interference” as used herein refers to the use of agents that decrease the expression of a target gene by degradation of a target mRNA through endogenous gene silencing pathways (e.g., Dicer and RNA-induced silencing complex (RISC)). RNA interference may be accomplished using various agents, including shRNA and siRNA. “Short hair-pin RNA” or “shRNA” refers to a double stranded, artificial RNA molecule with a hairpin turn that can be used to silence target gene expression via RNA interference (RNAi). Expression of shRNA in cells is typically accomplished by delivery of plasmids or through viral or bacterial vectors. shRNA is an advantageous mediator of RNAi in that it has a relatively low rate of degradation and turnover. Small interfering RNA (siRNA) is a class of double-stranded RNA molecules, usually 20-25 base pairs in length, similar to miRNA, and operating within the RNA interference (RNAi) pathway. It interferes with the expression of specific genes with complementary nucleotide sequences by degrading mRNA after transcription, preventing translation. In certain embodiments, an siRNA is 18, 19, 20, 21, 22, 23 or 24 nucleotides in length and has a 2 base overhang at its 3′ end. siRNAs can be introduced to an individual cell and/or culture system and result in the degradation of target mRNA sequences. “Morpholino” as used herein refers to a modified nucleic acid oligomer wherein standard nucleic acid bases are bound to morpholine rings and are linked through phosphorodiamidate linkages. Similar to siRNA and shRNA, morpholinos bind to complementary mRNA sequences. However, morpholinos function through steric-inhibition of mRNA translation and alteration of mRNA splicing rather than targeting complementary mRNA sequences for degradation.

The terms “treatment”, “treating” and the like are used herein to generally mean obtaining a desired pharmacologic and/or physiologic effect. The effect may be prophylactic in terms of completely or partially preventing a disease or symptom thereof, e.g. reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The practice of the present invention will employ, unless otherwise indicated, conventional techniques of cell biology, molecular biology techniques), microbiology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991), each of which is expressly incorporated by reference herein.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

All publications mentioned herein are incorporated herein by reference to disclose and describe the methods and/or materials in connection with which the publications are cited. It is understood that the present disclosure supersedes any disclosure of an incorporated publication to the extent there is a contradiction.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Unless otherwise indicated, all terms used herein have the same meaning as they would to one skilled in the art and the practice of the present invention will employ, conventional techniques of microbiology and recombinant DNA technology, which are within the knowledge of those of skill of the art.

### II. General

The present invention provides methods of modulating WNT signals to ameliorate gastrointestinal disorders, including but limited to, inflammatory bowel disease, including but not limited to, Crohn's disease, Crohn's disease with fistula formation, and ulcerative colitis. In particular the present invention provides a WNT/β-catenin signaling agonist to enhance regeneration of the intestinal epithelium as a result of injury from these disorders.

WNT (“Wingless-related integration site” or “Wingless and Int-1” or “Wingless-Int”) ligands and their signals play key roles in the control of development, homeostasis and regeneration of many essential organs and tissues, including bone, liver, skin, stomach, intestine, kidney, central nervous system, mammary gland, taste bud, ovary, cochlea, lung, and many other tissues (reviewed, e.g., by Clevers, Loh, and Nusse, 2014; 346:1248012). Modulation of WNT signaling pathways has potential for treatment of degenerative diseases and tissue injuries.

One of the challenges for modulating WNT signaling as a therapeutic is the existence of multiple WNT ligands and WNT receptors, Frizzled 1-10 (FZD1-10), with many tissues expressing multiple and overlapping FZDs. Canonical WNT signals also involve Low-density lipoprotein (LDL) receptor-related protein 5 (LRP5) or Low-density lipoprotein (LDL) receptor-related protein 6 (LRP6) as co-receptors, which are broadly expressed in various tissues, in addition to FZDs.

R-spondins 1˜4 are a family of ligands that amplify WNT signals. Each of the R-spondins work through a receptor complex that contains Zinc and Ring Finger 3 (ZNRF3) or Ring Finger Protein 43 (RNF43) on one end and a Leucine-rich repeat-containing G-protein coupled receptor 4-6 (LGR4-6) on the other (reviewed, e.g., by Knight and Hankenson 2014, Matrix Biology; 37: 157-161). R-spondins might also work through additional mechanisms of action. ZNRF3 and RNF43 are two membrane-bound E3 ligases specifically targeting WNT receptors (FZD1-10 and LRP5 or LRP6) for degradation. Binding of an R-spondin to ZNRF3/RNF43 and LGR4-6 causes clearance or sequestration of the ternary complex, which removes E3 ligases from WNT receptors and stabilizes WNT receptors, resulting in enhanced WNT signals. Each R-spondin contains two Furin domains (1 and 2), with Furin domain 1 binding to ZNRF3/RNF43, and Furin domain 2 binding to LGR4-6. Fragments of R-spondins containing Furin domains 1 and 2 are sufficient for amplifying WNT signaling. While R-spondin effects depend on WNT signals, since both LGR4-6 and ZNRF3/RNF43 are widely expressed in various tissues, the effects of R-spondins are not tissue-specific.

Activating WNT signaling by RSPO or by a WNT agonist may be used for the treatment of gastrointestinal disorders. Previous work in the literature suggests RSPO may be used for the treatment of experimental colon colitis (J. Zhao et. al., 2007). A WNT agonist molecule may also be used for the treatment of gastrointestinal disorders. In particular, active WNT signaling can provide a major stem cell maintenance signal and plays a key role in regulating regeneration of the intestinal epithelium in homeostasis and in injury. The two intestinal epithelial lineages, absorptive and secretory, define the two main functions of the gut apparatus: secretory cells secrete hormones and provide an important barrier against food-borne microorganisms, toxins, and antigens, mainly through the secretion of mucus and anti-microbial peptides. In contrast, the absorptive cells conduct uptake of dietary nutrients, as they localize mainly at the tips of the villi in the small intestine or at the top of the colonic crypts, thus constituting the majority of luminal cells across the intestinal surface area (see, e.g., Santos, et. al (2018) Trends in Cell Biol. in press, https://doi.org/10.1016/j.tcb.2018.08.001). Under homeostasis conditions, all cells in the intestinal epithelium regenerate in 3-10 days.

Different niche factors maintain ISC activity and distinct non-epithelial and/or epithelial cells elaborate various signals that make up a cellular niche. Such niche factors include canonical signals such as WNT, R-spondin, Notch, and Bone Morpohogenetic Protein (BMP), but also inflammatory and dietary influences. Upon injury, the ISC niche adapts beyond its homeostatic state to interpret pathogenic stimuli and translate them into regeneration of the epithelium. This regeneration is mediated by either surviving Lgr5+ISCs or other mature cell types such as enterocytes, enteroendocrine, or Paneth cells that can convert back to Lgr5+ISCs to aid epithelial regeneration (Beumer and Clevers (2016), Development 143: 3639-3649).

Intestinal Stem Cells (ISCs) at the bottom of the intestinal crypt, also known as columnar base cells (CBCs), are intercalated with WNT secreting Paneth cells (Cheng and Leblond (1974) Am. J. Anat. 141: 537-561). Mesenchymal cells surrounding the intestinal epithelium also secret some WNT proteins, serving an overlapping stem cell niche function in vivo (Farin, el. al (2012) Gastroenterol. 143: 1518-1529). In the presence of WNT signaling, ISCs divide to produce self-renewing stem cells and differentiating daughter cells which first go through a few fast transit amplifying (TA) divisions before differentiating into functional cell types. There is also a quiescent stem cell population in the intestinal crypt, +4 cells, which can contribute to epithelial regeneration when CBCs are damaged (Tian, el. al (2011) Nature 478: 255-259). Commitment to individual lineage and terminal differentiation take place as the TA cells migrate out along the crypt-villus axis, away from the WNT producing cells.

In some embodiments, the WNT/β-catenin signaling agonist can include binding agents or epitope binding domains that bind one or more FZD receptors and inhibit or enhance WNT signaling. In certain embodiments, the agent or antibody specifically binds to the cysteine-rich domain (CRD) within the human frizzled receptor(s) to which it binds. Additionally, binding agents containing epitope binding domains against LRP can also be used. In some embodiments, the WNT/β-catenin agonist possesses binding agents or epitope binding domains that bind E3 ligases ZNRF3/RNF43. The E3 ligase agonist antibodies or fragments thereof can be single molecules or combined with other WNT antagonists, e.g., FZD receptor antagonists, LRP receptor antagonists, etc.

As is well known in the art, an antibody is an immunoglobulin molecule capable of specific binding to a target such as a carbohydrate, polynucleotide, lipid, polypeptide, etc., through at least on epitope binding domain, located on the variable region of the immunoglobulin molecule. As used herein, the term encompasses not only intact polyclonal or monoclonal antibodies, but also fragments thereof containing epitope binding domains (e.g., dAb, Fab, Fab′, (F(ab′)2, Fv, single chain (scFv), Nanobodies® (Nabs; also known as sdAbs or VHH domains), DVD-Igs, synthetic variants thereof, naturally occurring variants, fusion proteins comprising and epitope binding domain, humanized antibodies, chimeric antibodies, and any other modified configuration of the immunoglobulin molecule that comprises an antigen-binding site or fragment (epitope recognition site) of the required specificity. “Diabodies,” multivalent or multispecific fragments constructed by gene fusion (WO94/13804; P. Holliger et al., Proc. Natl. Acad. Sci. USA 90 6444-6448, 1993) are also a particular form of antibody contemplated herein. Minibodies comprising a scFv joined to a CH3 domain are also included herein (S. Hu et al., Cancer Res., 56, 3055-3061, 1996). See e.g., Ward, E. S. et al., Nature 341, 544-546 (1989); Bird et al., Science, 242, 423-426, 1988; Huston et al., PNAS USA, 85, 5879-5883, 1988); PCT/US92/09965; WO94/13804; P. Holliger et al., Proc. Natl. Acad. Sci. USA 90 6444-6448, 1993; Y. Reiter et al., Nature Biotech, 14, 1239-1245, 1996; S. Hu et al., Cancer Res., 56, 3055-3061, 1996.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv fragment for use according to certain embodiments of the present disclosure can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH::VL heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

In certain embodiments, single chain Fv or scFV antibodies are contemplated. For example, Kappa bodies (Ill et al., Prot. Eng. 10: 949-57 (1997)); minibodies (Martin et al., EMBO J 13: 5305-9 (1994)); diabodies (Holliger et al., PNAS 90: 6444-8 (1993)); or Janusins (Traunecker et al., EMBO J 10: 3655-59 (1991) and Traunecker et al., Int. J. Cancer Suppl. 7: 51-52 (1992)), may be prepared using standard molecular biology techniques following the teachings of the present application with regard to selecting antibodies having the desired specificity. In still other embodiments, bispecific or chimeric antibodies may be made that encompass the ligands of the present disclosure. For example, a chimeric antibody may comprise CDRs and framework regions from different antibodies, while bispecific antibodies may be generated that bind specifically to one or more FZD receptors through one binding domain and to a second molecule through a second binding domain. These antibodies may be produced through recombinant molecular biological techniques or may be physically conjugated together.

A single chain Fv (scFv) polypeptide is a covalently linked VH::VL heterodimer which is expressed from a gene fusion including VH- and VL-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an scFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In certain embodiments, an antibody as described herein is in the form of a diabody. Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associate with each other to form an antigen binding site: antigen binding sites are formed by the association of the first domain of one polypeptide within the multimer with the second domain of another polypeptide within the multimer (WO94/13804).

A dAb fragment of an antibody consists of a VH domain (Ward, E. S. et al., Nature 341, 544-546 (1989)).

Where bispecific antibodies are to be used, these may be conventional bispecific antibodies, which can be manufactured in a variety of ways (Holliger, P. and Winter G., Current Opinion Biotechnol. 4, 446-449 (1993)), e.g., prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knob s-into-holes engineering (J. B. B. Ridgeway et al., Protein Eng., 9, 616-621 (1996)).

In certain embodiments, the antibodies described herein may be provided in the form of a UniBody®. A UniBody® is an IgG4 antibody with the hinge region removed (see GenMab Utrecht, The Netherlands; see also, e.g., US20090226421). This proprietary antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells.

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are

denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (scFv), Nanobodies®, variants thereof, fusion proteins comprising an antigen-binding fragment of a monoclonal antibody, humanized monoclonal antibodies, chimeric monoclonal antibodies, and any other modified configuration of the immunoglobulin molecule that comprises an antigen-binding fragment (epitope recognition site) of the required specificity and the ability to bind to an epitope, including WNT surrogate molecules disclosed herein. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv fragment for use according to certain embodiments of the present disclosure can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH::VL heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

In certain embodiments, single chain Fv or scFV antibodies are contemplated. For example, Kappa bodies (Ill et al., Prot. Eng. 10: 949-57 (1997)); minibodies (Martin et al., EMBO J 13: 5305-9 (1994)); diabodies (Holliger et al., PNAS 90: 6444-8 (1993)); or Janusins (Traunecker et al., EMBO J 10: 3655-59 (1991) and Traunecker et al., Int. J. Cancer Suppl. 7: 51-52 (1992)), may be prepared using standard molecular biology techniques following the teachings of the present application with regard to selecting antibodies having the desired specificity. In still other embodiments, bispecific or chimeric antibodies may be made that encompass the ligands of the present disclosure. For example, a chimeric antibody may comprise CDRs and framework regions from different antibodies, while bispecific antibodies may be generated that bind specifically to one or more FZD receptors through one

binding domain and to a second molecule through a second binding domain. These antibodies may be produced through recombinant molecular biological techniques or may be physically conjugated together.

A single chain Fv (scFv) polypeptide is a covalently linked VH::VL heterodimer which is expressed from a gene fusion including VH- and VL-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an scFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In certain embodiments, an antibody as described herein is in the form of a diabody. Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associate with each other to form an antigen binding site: antigen binding sites are formed by the association of the first domain of one polypeptide within the multimer with the second domain of another polypeptide within the multimer (WO94/13804).

A dAb fragment of an antibody consists of a VH domain (Ward, E. S. et al., Nature 341, 544-546 (1989)). Where bispecific antibodies are to be used, these may be conventional bispecific antibodies, which can be manufactured in a variety of

ways (Holliger, P. and Winter G., Current Opinion Biotechnol. 4, 446-449 (1993)), e.g., prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knob s-into-holes engineering (J. B. B. Ridgeway et al., Protein Eng., 9, 616-621 (1996)).

In certain embodiments, the antibodies described herein may be provided in the form of a UniBody®. A UniBody® is an IgG4 antibody with the hinge region removed (see GenMab Utrecht, The Netherlands; see also, e.g., US20090226421). This proprietary antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells.

In certain embodiments, the antibodies of the present disclosure may take the form of a single domain (sdAb) or VHH antibody fragment (also known as a Nanobody®). The sdAb or VHH technology was originally developed following the discovery and identification that camelidae (e.g., camels and llamas) possess fully functional antibodies that consist of heavy chains only and therefore lack light chains. These heavy-chain only antibodies contain a single variable domain (VHH) and two constant domains (CH2, CH3). The cloned and isolated single variable domains have full antigen binding capacity and are very stable. These single variable domains, with their unique structural and functional properties, form the basis of “Nanobodies®”. The sdAbs or VHHs are encoded by single genes and are efficiently produced in almost all prokaryotic and eukaryotic hosts, e.g., E. coli (see, e.g., U.S. Pat. No. 6,765,087), molds (for example Aspergillus or Trichoderma) and yeast (for example Saccharomyces, Kluyvermyces, Hansenula or Pichia (see, e.g., U.S. Pat. No. 6,838,254). The production process is scalable and multi-kilogram quantities of Nanobodies® have been produced. sdAbs or VHHs may be formulated as a ready-to-use solution having a long shelf life. The Nanoclone® method (see, e.g., WO 06/079372) is a proprietary method for generating Nanobodies® against a desired target, based on automated high-throughput selection of B-cells. sdAbs or VHHs are single-domain antigen-binding fragments of camelid-specific heavy-chain only antibodies.

Another antibody fragment contemplated is a dual-variable domain-immunoglobulin (DVD-Ig) is an engineered protein that combines the function and specificity of two monoclonal antibodies in one molecular entity. A DVD-Ig is designed as an IgG-like molecule, except that each light chain and heavy chain contains two variable domains in tandem through a short peptide linkage, instead of one variable domain in IgG. The fusion orientation of the two variable domains and the choice of linker sequence are critical to functional activity and efficient expression of the molecule. A DVD-Ig can be produced by conventional mammalian expression systems as a single species for manufacturing and purification. A DVD-Ig has the specificity of the parental antibodies, is stable in vivo, and exhibits IgG-like physicochemical and pharmacokinetic properties. DVD-Igs and methods for making them are described in Wu, C., et al., Nature Biotechnology, 25:1290-1297 (2007)).

In certain embodiments, the antibodies or antigen-binding fragments thereof as disclosed herein are humanized. This refers to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio, A. F. et al., (1989) Proc Natl Acad Sci USA 86:4220-4224; Queen et al., PNAS (1988) 86:10029-10033; Riechmann et al., Nature (1988) 332:323-327). Illustrative methods for humanization of the anti-FZD or LRP antibodies disclosed herein include the methods described in U.S. Pat. No. 7,462,697.

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato, K., et al., (1993) Cancer Res 53:851-856; Riechmann, L., et al., (1988) Nature 332:323-327; Verhoeyen, M., et al., (1988) Science 239:1534-1536; Kettleborough, C. A., et al., (1991) Protein Engineering 4:773-3783; Maeda, H., et al., (1991) Human Antibodies Hybridoma 2:124-134; Gorman, S. D., et al., (1991) Proc Natl Acad Sci USA 88:4181-4185; Tempest, P. R., et al., (1991) Bio/Technology 9:266-271; Co, M. S., et al., (1991) Proc Natl Acad Sci USA 88:2869-2873; Carter, P., et al., (1992) Proc Natl Acad Sci USA 89:4285-4289; and Co, M. S. et al., (1992) J Immunol 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies of the present disclosure may be chimeric antibodies. In this regard, a chimeric antibody is comprised of an antigen-binding fragment of an antibody operably linked or otherwise fused to a heterologous Fc portion of a different antibody. In certain embodiments, the heterologous Fc domain is of human origin. In other embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

The structures and locations of immunoglobulin CDRs and variable domains may be determined by reference to Kabat, E. A. et al., Sequences of Proteins of Immunological Interest. 4th Edition. US Department of Health and Human Services. 1987, and updates thereof, now available on the Internet (immuno.bme.nwu.edu).

In some embodiments, WNT surrogate molecule comprises one or more Fab or antigen-binding fragment thereof and one or more VHH or sdAb or antigen-binding fragment thereof (or alternatively, one or more scFv or antigen-binding fragment thereof). In certain embodiments, the Fab specifically binds one or more Fzd receptor, and the VHH or sdAb (or scFv) specifically binds LRPS and/or LRP6. In certain embodiments, the Fab specifically binds LRPS and/or LRP6, and the VHH or sdAb (or scFv) specifically binds one or more Fzd receptor. In certain embodiments, the VHH or sdAb (or scFv) is fused to the N-terminus of the Fab, while in some embodiments, the VHH or sdAb (or scFv) is fused to the C-terminus of the Fab. In particular embodiments, the Fab is present in a full IgG format, and the VHH or sdAb (or scFv) is fused to the N-terminus and/or C-terminus of the IgG light chain. In particular embodiments, the Fab is present in a full IgG format, and the VHH or sdAb (or scFv) is fused to the N-terminus and/or C-terminus of the IgG heavy chain. In particular embodiments, two or more VHH or sdAb (or scFvs) are fused to the IgG at any combination of these locations.

Fabs may be converted into a full IgG format that includes both the Fab and Fc fragments, for example, using genetic engineering to generate a fusion polypeptide comprising the Fab fused to an Fc region, i.e., the Fab is present in a full IgG format. The Fc region for the full IgG format may be derived from any of a variety of different Fcs, including but not limited to, a wild-type or modified IgG1, IgG2, IgG3, IgG4 or other isotype, e.g., wild-type or modified human IgG1, human IgG2, human IgG3, human IgG4, human IgG4Pro (comprising a mutation in core hinge region that prevents the formation of IgG4 half molecules), human IgA, human IgE, human IgM, or the modified IgG1 referred to as IgG1 LALAPG. The L235A, P329G (LALA-PG) variant has been shown to eliminate complement binding and fixation as well as Fc-γ dependent antibody-dependent cell-mediated cytotoxity (ADCC) in both murine IgG2a and human IgG1. These LALA-PG substitutions allow a more accurate translation of results generated with an “effectorless” antibody framework scaffold between mice and primates. In particular embodiments of any of the IgG disclosed herein, the IgG comprises one or more of the following amino acid substitutions: N297G, N297A, N297E, L234A, L235A, or P236G.

Non-limiting examples of bivalent and bispecific WNT surrogate molecules that are bivalent towards both the one or more Fzd receptor and the LRP5 and/or LRP6 are provided. The VHH or sdAb (or scFvs) may be fused to the N-termini of both light chains, to the N-termini of both heavy chains, to the C-termini of both light chains, or to the C-termini of both heavy chains. It is further contemplated, e.g., that VHH or sdAb (or scFvs) could be fused to both the N-termini and C-termini of the heavy and/or light chains, to the N-termini of the light chains and the heavy chains, to the C-termini of the heavy and light chains, to the N-termini of the heavy chains and C-termini of the light chains, or to the C-termini of the heavy chains and the N-termini of the light chains. In other related embodiments, two or more VHH or sdAb (or scFvs) may be fused together, optionally via a linker moiety, and fused to the Fab or IgG at one or more of these locations. In a related embodiment, the WNT surrogate molecule has a Hetero-IgG format, whereas the Fab is present as a half antibody, and one or more VHH or sdAb (or scFv) is fused to one or more of the N-terminus of the Fc, the N-terminus of the Fab, the C-terminus of the Fc, or the C-terminus of the Fab. In certain embodiments, the Fab or antigen-binding fragment (or IgG) thereof is fused directly to the VHH or sdAb (or scFv) or antigen-binding fragment thereof, whereas in other embodiments, the binding regions are fused via a linker moiety.

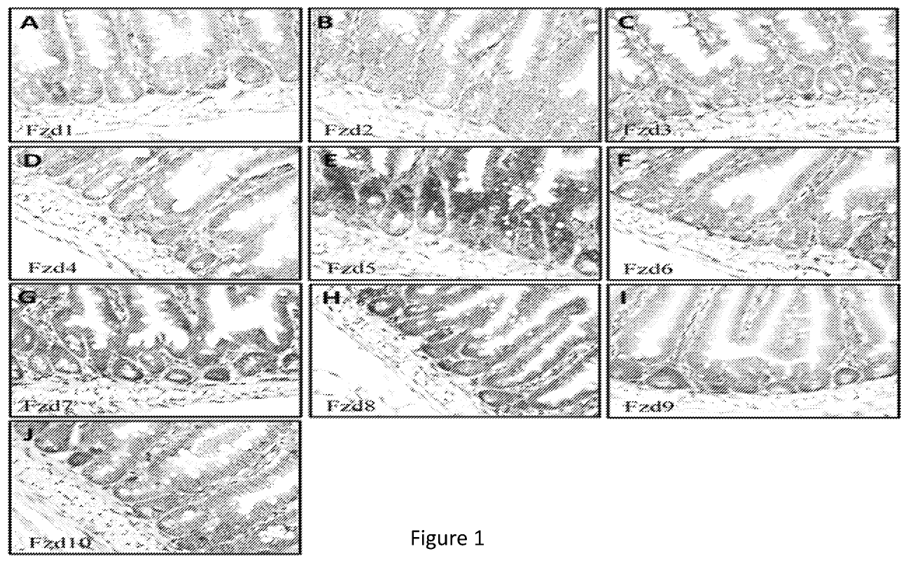

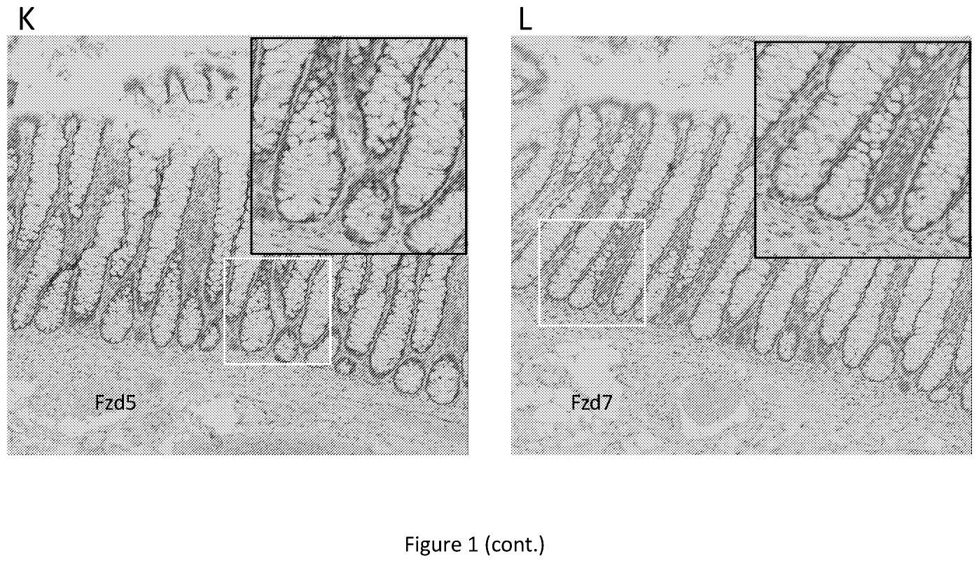

In various embodiments, a WNT surrogate molecule comprises one or more Fab or antigen-binding fragment thereof that binds one or more FZD receptor and one or more Fab or antigen-binding fragment thereof that binds LRP5 and/or LRP6. In certain embodiments, it comprises two Fab or antigen-binding fragments thereof that bind one or more FZD receptor and/or two Fab or antigen-binding fragments thereof that bind LRP5 and/or LRP6. In particular embodiments, one or more of the Fab is present in a full IgG format, and in certain embodiments, both Fab are present in a full IgG format. In certain embodiments, the Fab in full IgG format specifically binds one or more FZD receptor, and the other Fab specifically binds LRP5 and/or LRP6. In certain embodiments, the Fab specifically binds one or more FZD receptor, and the Fab in full IgG format specifically binds LRP5 and/or LRP6. In certain embodiments, the Fab specifically binds LRP5 and/or LRP6, and the Fab in full IgG format specifically binds one or more FZD receptor. In certain embodiments, the Fab is fused to the N-terminus of the IgG, e.g., to the heavy chain or light chain N-terminus, optionally via a linker. In certain embodiments, the Fab is fused to the N-terminus of the heavy chain of the IgG and not fused to the light chain. In particular embodiments, the two heavy chains can be fused together directly or via a linker. An example of such a bispecific and bivalent with respect to both receptors is shown at the top of FIG. 1B. In other related embodiments, two or more VHH or sdAb may be fused together, optionally via a linker moiety, and fused to the Fab or IgG at one or more of these locations. In a related embodiment, the WNT surrogate molecule has a Hetero-IgG format, whereas one of the Fab is present as a half antibody, and the other Fab is fused to one or more of the N-terminus of the Fc, the N-terminus of the Fab, or the C-terminus of the Fc. In certain embodiments, the Fab or antigen-binding fragment thereof is fused directly to the other Fab or IgG or antigen-binding fragment thereof, whereas in other embodiments, the binding regions are fused via a linker moiety.

In certain embodiments, the WNT agonists of the present invention can have, comprise, or consist of any of the sequences provided in Table 2, Table 4, Table 5, Table 6, or Table 7, or functional fragments or variants thereof.

In certain embodiments, the antagonist or agonist binding agent binds with a dissociation constant (K_{D}) of about 1 μM or less, about 100 nM or less, about 40 nM or less, about 20 nM or less, or about 10 nM or less. For example, in certain embodiments, a FZD binding agent or antibody described herein that binds to more than one FZD, binds to those FZDs with a K_{D} of about 100 nM or less, about 20 nM or less, or about 10 nM or less. In certain embodiments, the binding agent binds to one or more its target antigen with an EC50 of about 1 μM or less, about 100 nM or less, about 40 nM or less, about 20 nM or less, about 10 nM or less, or about 1 nM 20 or less.

The antibodies or other agents of the present invention can be assayed for specific binding by any method known in the art. The immunoassays which can be used include, but are not limited to, competitive and non-competitive assay systems using techniques such as BIAcore analysis, FACS analysis, immunofluorescence, immunocytochemistry, Western blots, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety).

For example, the specific binding of an antibody to a target antigen may be determined using ELISA. An ELISA assay comprises preparing antigen, coating wells of a 96 well microtiter plate with antigen, adding the antibody or other binding agent conjugated to a detectable compound such as an enzymatic substrate (e.g. horse-radish peroxidase or alkaline phosphatase) to the well, incubating for a period of time and detecting the presence of the antigen. In some embodiments, the antibody or agent is not conjugated to a detectable compound, but instead a second conjugated antibody that recognizes the first antibody or agent is added to the well. In some embodiments, instead of coating the well with the antigen, the antibody or agent can be coated to the well and a second antibody conjugated to a detectable compound can be added following the addition of the antigen to the coated well. One of skill in the art would be knowledgeable as to the parameters that can be modified to increase the signal detected as well as other variations of ELISAs known in the art (see e.g. Ausubel et al, eds, 1994, Current Protocols in Molecular Biology, Vol. 1, John Wiley & Sons, Inc., New York at 11.2.1).

The binding affinity of an antibody or other agent to a target antigen and the off-rate of the antibody-antigen interaction can be determined by competitive binding assays. One example of a competitive binding assay is a radioimmunoassay comprising the incubation of labeled antigen (e.g., FZD, LRP), or fragment or variant thereof, with the antibody of interest in the presence of increasing amounts of unlabeled antigen followed by the detection of the antibody bound to the labeled antigen. The affinity of the antibody and the binding off-rates can be determined from the data by scatchard plot analysis. In some embodiments, BIAcore kinetic analysis is used to determine the binding on and off rates of antibodies or agents. BIAcore kinetic analysis comprises analyzing the binding and dissociation of antibodies from chips with immobilized antigens on their surface.

WNT surrogate molecules of the present invention are biologically active in binding to one or more FZD receptor and to one or more of LRP5 and LRP6, and in activation of WNT signaling, i.e., the WNT surrogate molecule is a WNT agonist. The term “WNT agonist activity” refers to the ability of an agonist to mimic the effect or activity of a WNT protein binding to a frizzled protein and/or LRP5 or LRP6. The ability of the WNT surrogate molecules and other WNT agonists disclosed herein to mimic the activity of WNT can be confirmed by a number of assays. WNT agonists typically initiate a reaction or activity that is similar to or the same as that initiated by the receptor's natural ligand. In particular, the WNT agonists disclosed herein activate, enhance or increase the canonical WNT/β-catenin signaling pathway. As used herein, the term “enhances” refers to a measurable increase in the level of WNT/β-catenin signaling compared with the level in the absence of a WNT agonist, e.g., a WNT surrogate molecule disclosed herein. In particular embodiments, the increase in the level of WNT/β-catenin signaling is at least 10%, at least 20%, at least 50%, at least two-fold, at least five-fold, at least 10-fold, at least 20-fold, at least 50-fold, or at least 100-fold as compared to the level of WNT/β-catenin signaling in the absence of the WNT agonist, e.g., in the same cell type. Methods of measuring WNT/β-catenin signaling are known in the art and include those described herein.

In particular embodiments, WNT surrogate molecules disclosed herein are bispecific, i.e., they specifically bind to two or more different epitopes, e.g., one or more FZD receptor, and LRPS and/or LRP6. In certain embodiments the WNT surrogate molecules bind to FZD 5 and/or FZD 8, and LRPS and/or LRP6.

In particular embodiments, WNT surrogate molecules disclosed herein are multivalent, e.g., they comprise two or more regions that each specifically bind to the same epitope, e.g., two or more regions that bind to an epitope within one or more FZD receptor and/or two or more regions that bind to an epitope within LRPS and/or LRP6. In particular embodiments, they comprise two or more regions that bind to an epitope within one or more FZD receptor and two or more regions that bind to an epitope within LRPS and/or LRP6. In certain embodiments, WNT surrogate molecules comprise a ratio of the number of regions that bind one or more FZD receptor to the number of regions that bind LRPS and/or LRP6 of or about: 1:1, 2:1, 3:1, 4:1, 5:1, 6:1, 2:3, 2:5, 2:7, 7:2, 5:2, 3:2, 3:4, 3:5, 3:7, 3:8, 8:3, 7:3, 5:3, 4:3, 4:5, 4:7, 4:9, 9:4, 7:4, 5:4, 6:7, 7:6, 1:2, 1:3, 1:4, 1:5, or 1:6. In certain embodiments, WNT surrogate molecules are bispecific and multivalent.

In certain aspects, the present disclosure provides novel tissue-specific WNT signal enhancing molecules capable of enhancing WNT activity in a tissue- or cell-specific manner. In certain embodiments, the tissue-specific WNT signal enhancing molecules are bi-functional molecules comprising a first domain that binds to one or more ZNRF3 and/or RNF43 ligases, and a second domain that binds to one or more targeted tissue or cell type in a tissue- or cell-specific manner. Each of the first domain and the second domain may be any moiety capable of binding to the ligase complex or targeted tissue or cell, respectively. For example, each of the first domain and the second domain may be, but are not limited to, a moiety selected from: a polypeptide (e.g., an antibody or antigen-binding fragment thereof or a peptide or polypeptide different from an antibody), a small molecule, and a natural ligand or a variant, fragment or derivative thereof. In certain embodiments, the natural ligand is a polypeptide, a small molecule, an ion, an amino acid, a lipid, or a sugar molecule. The first domain and the second domain may be the same type of moiety as each other, or they may be different types of moieties. In certain embodiments, the tissue-specific WNT signal enhancing molecules bind to a tissue- or cell-specific cell surface receptor. In particular embodiments, the tissue-specific WNT signal enhancing molecules increase or enhance WNT signaling by at least 50%, at least two-fold, at least three-fold, at least five-fold, at least ten-fold, at least twenty-fold, at least thirty-fold, at least forty-fold, or at least fifty-fold, e.g., as compared to a negative control.

Tissue-specific WNT signal enhancing molecules may have different formats. In particular embodiments, the tissue-specific WNT signal enhancing molecules are fusion proteins comprising a first polypeptide sequence that binds to ZNRF3/RNF43 and a second polypeptide sequence that binds to one or more targeted tissue or cell type in a tissue- or cell-specific manner. In certain embodiments, the two polypeptide sequences may be fused directly or via a linker. In certain embodiments, the tissue-specific WNT signal enhancing molecules comprise two or more polypeptides, such as dimers or multimers comprising two or more fusion proteins, each comprising the first domain and the second domain, wherein the two or more polypeptides are linked, e.g., through a linker moiety or via a bond between amino acid residues in each of the two or more polypeptides, e.g., an intermolecular disulfide bond between cysteine residues.

In particular embodiments, a tissue-specific WNT signal enhancing molecule is an antibody comprising antibody heavy and light chains (or antigen-binding fragments thereof) that constitute either the first domain or the second domain, wherein the other domain (i.e., the second domain or first domain) is linked to the antibody heavy chain or light chain, either as a fusion protein or via a linker moiety. In particular embodiments, the other domain is linked to the N-terminus of the heavy chain, the C-terminus of the heavy chain, the N-terminus of the light chain, or the C-terminus of the light chain. Such structures may be referred to herein as appended IgG scaffolds or formats. For example, a tissue-specific WNT signal enhancing molecule can be an antibody that binds ZNRF3/RNF43, wherein a binding domain that binds a tissue- or cell-specific receptor is fused or appended to either the heavy chain or light chain of the antibody that binds ZNRF3/RNF43. In another example, a tissue-specific WNT signal enhancing molecule can be an antibody that binds a tissue- or cell-specific receptor, wherein a binding domain that binds ZNRF3/RNF43 is fused or appended to either the heavy chain or light chain of the antibody that binds the tissue- or cell-specific receptor.

In particular embodiments, an intestine-specific WNT signal enhancing molecule is an antibody or antigen-binding fragment thereof that binds GPA33, CDH17, MUC-13, wherein a binding domain that binds ZNRF3/RNF43 is fused or appended to either the heavy chain or light chain of the antibody or antigen-binding fragment thereof. In particular embodiments, the binding domain that bind ZNRF3/RNF43 comprises Fu1 and Fu2 domains, wherein the Fu1 and Fu2 domains optionally comprise one or more amino acid modifications, including any of those disclosed herein, e.g., F105R and/or F109A.

In certain embodiments, the tissue-specific WNT signal enhancing molecules comprise a first domain (“action module”) that binds ZNRF3/RNF43 and a second domain (“targeting module”) that binds a tissue- or cell-specific receptor, e.g., with high affinity. In certain embodiments, each of these two domains has substantially reduced activity or is inactive in enhancing WNT signals by itself. However, when the tissue-specific WNT signal enhancing molecules engage with target tissues that express the tissue-specific receptor, E3 ligases ZNRF3/RNF43 are recruited to a ternary complex with the tissue-specific receptors, leading them to be sequestered, and/or cleared from the cell surface via receptor-mediated endocytosis. The net result is to enhance WNT signals in a tissue-specific manner.

In certain embodiments, the action module is a binder to ZNRF3/RNF43 E3 ligases, and it can be designed based on R-spondins, e.g., R-spondins-1-4, including but not limited to human R-spondins-1-4. In certain embodiments, the action module is an R-spondin, e.g., a wild-type R-spondin-1-4, optionally a human R-spondin-1-4, or a variant or fragment thereof. In particular embodiments, it is a variant of any of R-spondins-1-4 having at least 80%, at least 85%, at least 90%, at least 95%, at least 98%, or at least 99% sequence identity to the corresponding wild-type R-spondin-1-4 sequence. In certain embodiments, the action module comprises or consists of a Furin domain 1 of an R-spondin, e.g., any of R-spondins 1-4, which bind ZNRF3/RNF43. Extended versions of Furin domain 1 (including, but not limited to, those with a mutated Furin domain 2 that no longer binds to LGR4-6 or has reduced binding to LGR4-6) or engineered antibodies or any other derivatives or any engineered polypeptides different from antibodies that are able to bind specifically to ZNRF3/RNF43 can also be used. In certain embodiments, the action module comprises one or more Furin domain 1 of an R-spondin.

In certain embodiments, the action module does not comprise a Furin domain 2 of an R-spondin, or it comprises a modified or variant Furin domain 2 of an R-spondin, e.g., a Furin domain 2 with reduced activity as compared to the wild-type Furin domain 2. In certain embodiments, an action module comprises a Furin domain 1 but not a Furin domain 2 of R-spondin. In certain embodiments, an action module comprises two or more Furin domain 1 or multimers of a Furin domain 1. The action domain may comprise one or more wild-type Furin domain 1 of an R-spondin. In particular embodiments, the action module comprises a modified or variant Furin domain 1 of an R-spondin that has increased activity, e.g., binding to ZNRF3/RNF43, as compared to the wild-type Furin domain 1. Variants having increased binding to ZNRF3/RNF43 may be identified, e.g., by screening a phage or yeast display library comprising variants of an R-spondin Furin domain 1. Peptides or polypeptides unrelated to R-spondin Furin domain 1 but with increased binding to ZNRF3/RNF43 may also be identified through screening. Action modules may further comprise additional moieties or polypeptide sequences, e.g., additional amino acid residues to stabilize the structure of the action module or tissue-specific WNT signal enhancing molecule in which it is present.

In further embodiments, the action module comprises another inhibitory moiety, such as a nucleic acid molecule, which reduces or prevents ZNRF3/RNF43 activity or expression, such as, e.g., an anti-sense oligonucleotide; a small interfering RNA (siRNA); a short hairpin RNA (shRNA); a microRNA (miRNA); or a ribozyme. As used herein, “antisense” refers to a nucleic acid sequence, regardless of length, that is complementary to a nucleic acid sequence. In certain embodiments, antisense RNA refers to single-stranded RNA molecules that can be introduced to an individual cell, tissue, or subject and results in decreased expression of a target gene through mechanisms that do not necessarily rely on endogenous gene silencing pathways. An antisense nucleic acid can contain a modified backbone, for example, phosphorothioate, phosphorodithioate, or others known in the art, or may contain non-natural internucleoside linkages. Antisense nucleic acid can comprise, e.g., locked nucleic acids (LNA). In particular embodiments, the other inhibitor moiety inhibits an activity of one or both of ZNRF3/RNF43, or it inhibits the gene, mRNA or protein expression of one or both of ZNRF3/RNF43. In certain embodiments, the inhibitory moiety is a nucleic acid molecule that binds to a ZNRF3/RNF43 gene or mRNA, or a complement thereof.

In certain embodiments, the targeting module specifically binds to a cell-specific surface molecule, e.g., a cell-specific surface receptor, and can be, e.g., natural ligands, antibodies, or synthetic chemicals. In particular embodiments, the cell-specific surface molecule is preferentially expressed on a target organ, tissue or cell type, e.g., an organ, tissue or cell type in which it is desirous to enhance WNT signaling, e.g., to treat or prevent a disease or disorder. In particular embodiments, the cell-specific surface molecule has increased or enhanced expression on a target organ, tissue or cell type, e.g., an organ, tissue or cell type in which it is desirous to enhance WNT signaling, e.g., to treat or prevent a disease or disorder, e.g., as compared to one or more other non-targeted organs, tissues or cell types. In certain embodiments, the cell-specific surface molecule is preferentially expressed on the surface of the target organ, tissue or cell type as compared to one or more other organ, tissue or cell types, respectively. For example, in particular embodiments, a cell surface receptor is considered to be a tissue-specific or cell-specific cell surface molecule if it is expressed at levels at least two-fold, at least five-fold, at least 10-fold, at least 20-fold, at least 30-fold, at least 40-fold, at least 50-fold, at least 100-fold, at least 500-fold, or at least 1000-fold higher in the target organ, tissue or cell than it is expressed in one or more, five or more, all other organs, tissues or cells, or an average of all other organs, tissue or cells, respectively. In certain embodiments, the tissue-specific or cell-specific cell surface molecule is a cell surface receptor, e.g., a polypeptide receptor comprising a region located within the cell surface membrane and an extracellular region to which the targeting module can bind. In various embodiments, the methods described herein may be practiced by specifically targeting cell surface molecules that are only expressed on the target tissue or a subset of tissues including the target tissue, or by specifically targeting cell surface molecules that have higher levels of expression on the target tissue as compared to all, most, or a substantial number of other tissues, e.g., higher expression on the target tissue than on at least two, at least five, at least ten, or at least twenty other tissues.

Tissue-specific and cell-specific cell surface receptors are known in the art. Examples of tissue- and cell-specific surface receptors include but are not limited to GPA33, CDH17, and MUC-13. In certain embodiments, the targeting module comprises an antibody or antigen-binding fragment thereof that specifically binds these intestine specific receptors.

In certain embodiments, components of the WNT surrogate and WNT signal enhancing molecules may be combined to confer more tissue specificity.

### III. Pharmaceutical Compositions

Pharmaceutical compositions comprising a WNT agonist molecule described herein and one or more pharmaceutically acceptable diluent, carrier, or excipient are also disclosed.

In further embodiments, pharmaceutical compositions comprising a polynucleotide comprising a nucleic acid sequence encoding a WNT agonist molecule described herein and one or more pharmaceutically acceptable diluent, carrier, or excipient are also disclosed. In certain embodiments, the polynucleotides are DNA or mRNA, e.g., a modified mRNA. In particular embodiments, the polynucleotides are modified mRNAs further comprising a 5′ cap sequence and/or a 3′ tailing sequence, e.g., a polyA tail. In other embodiments, the polynucleotides are expression cassettes comprising a promoter operatively linked to the coding sequences.

In some embodiments the WNT agonist is an engineered recombinant polypeptide incorporating various epitope binding fragments that bind to various molecules in the WNT signaling pathway. For example The FZD and LRP antibody fragments (e.g., Fab, scFv, sdAbs, VHH, etc) may be joined together directly or with various size linkers, on one molecule. Similarly, a polypeptide such as RSPO, may be engineered to contain an antibody or fragment thereof against a tissue specific cell surface antigen, e.g, MUC-13. RSPO may also be administered concurrently or sequentially with an enhancer of the E3 ligases, ZNRF3/RNF43. The E3 ligase enhancer may be an agonist antibody or fragment that binds ZNRF3/RNF43 and enhances the E3 ligase activity.

Conversely, WNT agonists can also be recombinant polypeptides incorporating epitope binding fragments that bind to various molecules in the WNT signaling pathway and enhance WNT signaling. For example, a WNT agonist can be an antibody or fragment thereof that binds to FZD receptor and/or an LRP receptor and enhances WNT signaling. The FZD and LRP antibody fragments (e.g., Fab, scFv, sdAbs or VHHs, etc) may be joined together directly or with various size linkers, on one molecule.

In further embodiments, pharmaceutical compositions comprising an expression vector, e.g., a viral vector, comprising a polynucleotide comprising a nucleic acid sequence encoding a WNT agonist molecule described herein and one or more pharmaceutically acceptable diluent, carrier, or excipient are also disclosed.

The present disclosure further contemplates a pharmaceutical composition comprising a cell comprising an expression vector comprising a polynucleotide comprising a promoter operatively linked to a nucleic acid encoding a WNT agonist molecule and one or more pharmaceutically acceptable diluent, carrier, or excipient. In particular embodiments, the pharmaceutical composition further comprises a cell comprising an expression vector comprising a polynucleotide comprising a promoter operatively linked to a nucleic acid sequence encoding a WNT agonist. In particular embodiments, the cell is a heterologous cell or an autologous cell obtained from the subject to be treated.

The subject molecules, alone or in combination, can be combined with pharmaceutically-acceptable carriers, diluents, excipients and reagents useful in preparing a formulation that is generally safe, non-toxic, and desirable, and includes excipients that are acceptable for mammalian, e.g., human or primate, use. Such excipients can be solid, liquid, semisolid, or, in the case of an aerosol composition, gaseous. Examples of such carriers, diluents and excipients include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfate; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide. In particular embodiments, the pharmaceutical compositions are sterile.

Pharmaceutical compositions may further include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In some cases, the composition is sterile and should be fluid such that it can be drawn into a syringe or delivered to a subject from a syringe. In certain embodiments, it is stable under the conditions of manufacture and storage and is preserved against the contaminating action of microorganisms such as bacteria and fungi. The carrier can be, e.g., a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the WNT agonist antibody or antigen-binding fragment thereof (or encoding polynucleotide or cell comprising the same) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the pharmaceutical compositions are prepared with carriers that will protect the antibody or antigen-binding fragment thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers. These can be prepared according to methods known to those skilled in the art.

It may be advantageous to formulate the pharmaceutical compositions in dosage unit form for ease of administration and uniformity of dosage. Dosage unit form as used herein refers to physically discrete units suited as unitary dosages for the subject to be treated; each unit containing a predetermined quantity of active antibody or antigen-binding fragment thereof calculated to produce the desired therapeutic effect in association with the required pharmaceutical carrier. The specification for the dosage unit forms are dictated by and directly dependent on the unique characteristics of the antibody or antigen-binding fragment thereof and the particular therapeutic effect to be achieved, and the limitations inherent in the art of compounding such an active antibody or antigen-binding fragment thereof for the treatment of individuals.

The pharmaceutical compositions can be included in a container, pack, or dispenser, e.g. syringe, e.g. a prefilled syringe, together with instructions for administration.

The pharmaceutical compositions of the present disclosure encompass any pharmaceutically acceptable salts, esters, or salts of such esters, or any other compound which, upon administration to an animal comprising a human, is capable of providing (directly or indirectly) the biologically active antibody or antigen-binding fragment thereof.

The present disclosure includes pharmaceutically acceptable salts of a WNT agonist molecule described herein. The term “pharmaceutically acceptable salt” refers to physiologically and pharmaceutically acceptable salts of the compounds of the present disclosure: i.e., salts that retain the desired biological activity of the parent compound and do not impart undesired toxicological effects thereto. A variety of pharmaceutically acceptable salts are known in the art and described, e.g., in “Remington's Pharmaceutical Sciences”, 17th edition, Alfonso R. Gennaro (Ed.), Mark Publishing Company, Easton, Pa., USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66:2 (1977). Also, for a review on suitable salts, see “Handbook of Pharmaceutical Salts: Properties, Selection, and Use” by Stahl and Wermuth (Wiley-VCH, 2002). Pharmaceutically acceptable base addition salts are formed with metals or amines, such as alkali and alkaline earth metals or organic amines.

Metals used as cations comprise sodium, potassium, magnesium, calcium, and the like. Amines comprise N—N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present disclosure.

In some embodiments, the pharmaceutical composition provided herein comprise a therapeutically effective amount of a WNT agonist molecule or pharmaceutically acceptable salt thereof in admixture with a pharmaceutically acceptable carrier, diluent and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least six months at 4° C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer may be in the range of 6.5 to 7.75, preferably 7 to 7.5, and most preferably 7.2 to 7.4.

### V. Methods of Use

The present disclosure also provides methods for using the WNT agonist molecules and/or tissue-specific WNT signal enhancing molecules, e.g., to modulate a WNT signaling pathway, e.g., to increase WNT signaling, and the administration of a WNT agonist molecule and/or tissue-specific WNT signal enhancing molecule in a variety of therapeutic settings. Provided herein are methods of treatment using a WNT agonist molecule and/or a tissue-specific WNT signal enhancing molecule. Any of the methods disclosed herein may also be practiced using a combination of a WNT agonist molecule and a tissue-specific WNT signal enhancing molecule or a combination molecule comprising both a WNT agonist molecule and a tissue-specific WNT signal enhancing (combination molecule), e.g., as described herein. In one embodiment, a WNT agonist molecule and/or a tissue-specific WNT signal enhancing molecule, or combination molecule, is provided to a subject having a disease involving inappropriate or deregulated WNT signaling. In certain embodiments, methods disclosed herein comprise providing to a subject in need thereof a WNT agonist molecule and/or a tissue-specific WNT signal enhancing molecule, alone or in combination, or a combination molecule. In certain embodiments, a WNT agonist molecule and a tissue-specific WNT signal enhancing molecule are provided to the subject in the same or different pharmaceutical compositions. In some embodiments, the WNT agonist molecule and the tissue-specific WNT signal enhancing molecule are provided to the subject at the same time or at different times, e.g., either one before or after the other. In some embodiments, the methods comprise providing to the subject an effective amount of a WNT agonist molecule and/or tissue-specific WNT signal enhancing molecule. In some embodiments, an effective amount of the WNT agonist molecule and the tissue-specific WNT signal enhancing molecule are present in the subject during an overlapping time period, e.g., one day, two days, or one week. In other embodiments, methods disclosed herein comprise providing to a subject in need thereof a combination molecule comprising a WNT agonist molecule and a tissue-specific WNT signal enhancing molecule (combination molecule).

In certain embodiments, any of the methods disclosed herein may be practiced to reduce inflammation (e.g., inflammation associated with IBD or in a tissue affected by IBD, such as gastrointenstinal tract tissue, e.g., small intestine, large intestine, or colon), increase WNT signaling, reduce any of the histological symptoms of IBD (e.g., those disclosed herein), reduce cytokine levels in inflamed tissue (e.g., gastrointenstinal tract tissue), or reduce disease activity index as disclosed herein.

In certain embodiments, a WNT agonist molecule or tissue-specific WNT signal enhancing molecule or combination molecule may be used to enhance a WNT signaling pathway in a tissue or a cell. Agonizing the WNT signaling pathway may include, for example, increasing WNT signaling or enhancing WNT signaling in a tissue or cell. Thus, in some aspects, the present disclosure provides a method for agonizing a WNT signaling pathway in a cell, comprising contacting the tissue or cell with an effective amount of a WNT agonist molecule and/or a tissue-specific WNT signal enhancing molecule, or a combination molecule, or pharmaceutically acceptable salt thereof, disclosed herein, wherein the WNT agonist molecule and/or tissue-specific WNT signal enhancing molecule, or combination molecule is a WNT signaling pathway agonist. In some embodiments, contacting occurs in vitro, ex vivo, or in vivo. In particular embodiments, the cell is a cultured cell, and the contacting occurs in vitro.

The WNT agonist and/or tissue-specific WNT signal enhancing molecule, or combination molecule may be used for the treatment of gastrointestinal disorders, including but limited to, inflammatory bowel disease, including but not limited to, Crohn's disease, Crohn's disease with fistula formation, and ulcerative colitis. In particular the present invention provides a WNT/β-catenin signaling WNT/β-catenin agonist to enhance regeneration of the intestinal epithelium as a result of injury from these disorders.

In one embodiment, the WNT agonist molecule may also incorporate a tissue targeting moiety, e.g., an antibody or fragment thereof that recognizes a pulmonary tissue specific receptor or cell surface molecule.

The present invention also provides for combination treatment with known treatments gastrointestinal disorders, in particular inflammatory bowel diseases (IBD). For example, the WNT agonist and/or tissue-specific WNT signal enhancing molecule, or combination molecule can be combined with several known therapies for IBD, including, but not limited to, 5-Aminosalicylates (5-ASAs); immunosuppressants such as corticosteroids, azathioprine or 6-mercaptopurine, methotrexate, and ciclosporin-A or tacrolimus; TNFα inhibitors such as infliximab, adalimumab, and golimumab; anti-integrins such as vedolizumab; inflammatory cytokine antagonists such as ustekinumab; janus kinase (JAK) inhibitors such as tofacitinib; SMAD 7 inhibitors such as mongersen; and S1P modulators, such as ozanimod and etrasimod. The above therapeutic drugs can be administered sequentially or concurrently with the molecules of the present invention.

The therapeutic agent (e.g., a WNT agonist and/or tissue-specific WNT signal enhancing molecule or combination molecule) may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease. In some embodiments, the subject method results in a therapeutic benefit, e.g., preventing the development of a disorder, halting the progression of a disorder, reversing the progression of a disorder, etc. In some embodiments, the subject method comprises the step of detecting that a therapeutic benefit has been achieved. The ordinarily skilled artisan will appreciate that such measures of therapeutic efficacy will be applicable to the particular disease being modified, and will recognize the appropriate detection methods to use to measure therapeutic efficacy.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the present disclosure have been described herein for purposes of illustration, various modifications may be made without deviating from the spirit and scope of the present disclosure. Accordingly, the present disclosure is not limited except as by the appended claims.

The broad scope of this invention is best understood with reference to the following examples, which are not intended to limit the inventions to the specific embodiments.

### Examples

### I. General Methods

Standard methods in molecular biology are described. Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protocols in Immunology, Vol. 4, John Wiley, Inc., New York.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard methods of histology of the immune system are described. See, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.

Software packages and databases for determining, e.g., antigenic fragments, leader sequences, protein folding, functional domains, glycosylation sites, and sequence alignments, are available. See, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne et al. (2000) Bioinformatics 16: 741-742; Menne et al. (2000) Bioinformatics Applications Note 16:741-742; Wren et al. (2002) Comput. Methods Programs Biomed. 68:177-181; von Heijne (1983) Eur. J. Biochem. 133:17-21; von Heijne (1986) Nucleic Acids Res. 14:4683-4690.

### II. Expression of Frizzled Receptors in the Mouse Small Intestine and in Mouse and Human Colon

To determine expression pattern of each of the Frizzled receptors in the mouse small intestine and colon epithelium, mRNA for individual Frizzled receptors was detected by RNAscope® (ACD). RNAscope® probes used are listed in Table 1. Standard RNAscope® 2.5 HD Assay-Red protocol was followed.

TABLE 1

| ACD catelog # | Probes |
|---|---|
| 404871 | RNAscope ® Probe − Mm-FZD1 |
| 404881 | RNAscope ® Probe − Mm-FZD2 |
| 404891 | RNAscope ® Probe − Mm-FZD3 |
| 404901 | RNAscope ® Probe − Mm-FZD4 |
| 404911 | RNAscope ® Probe − Mm-FZD5 |
| 404921 | RNAscope ® Probe − Mm-FZD6 |
| 404931 | RNAscope ® Probe − Mm-FZD7 |
| 404941 | RNAscope ® Probe − Mm-FZD8 |
| 404951 | RNAscope ® Probe − Mm-FZD9 |
| 315781 | RNAscope ® Probe − Mm-FZD10 |

FZD1, FZD2, FZD3, FZD4, FZD5, FZD6, FZD7, FZD8, and FZD9 are expressed at different levels in the mouse intestinal epithelium (FIG. 1). FZD5 was expressed at the highest level in the intestinal crypts and villi. In the crypt, FZD 5 expression was much higher near the apical compartment where the Transit Amplifying (TA) cells reside. FZD1 was detected at low levels in both the intestinal epithelium and in lamina propria immediately surrounding intestinal crypts. FZD4, FZD6 and FZD7 were expressed at low levels and were evenly distributed in both the intestinal villi and crypts. Expression of FZD2, FZD3, FZD8, FZD9, and FZD10 was very low and was primarily detected in the intestinal crypts.

FZD1, FZD2, FZD3, FZD4, FZD5, FZD6, FZD7, FZD8, and FZD9 are also expressed at different levels in the mouse colon. FZD5 was expressed at the highest level in the colonic crypts, and the expression was higher towards the lumen side. FZD1 and FZD7 were detected at lower levels in the colon epithelium. FZD2, FZD3, FZD4, FZD 6, FZD 8 and FZD9 were expressed at low levels and were evenly distributed in colon crypts. There was no detectable expression of FZD10 in the intestine. Levels of FZD expression was affected in the colon of mouse DSS colitis IBD model and in human ulcerative colitis patient colon.

To determine expression pattern of each of the Frizzled receptors in the human colon epithelium, mRNA for individual Frizzled receptors was detected by RNAscope® (ACD). Standard RNAscope® 2.5 HD Assay-Red protocol was followed. FZD5 was expressed at the highest level in the colonic crypts. FZD7 was detected at lower levels in the colon epithelium and in the stromal cells encompassing the colon crypts.

### III. Activities of Engineered Soluble WNT Agonists

Activities of the three Frizzled biased WNT agonists, R2M3-26 (a FZD1, 2, 5, 7, 8 and LRP6 binder), 1RC07-03 (a FZD1, 2, 7 and LRP5 binder) and R2M13-03 (a FZD5, 8 and LRP5 binder), were examined in the human hepatic cell line, Huh7 cells to determine their ability to activate WNT signaling. The three WNT agonists were previously described in WO2019126398. Table 2 provides the sequences of the LC and HC chains of the three WNT agonists used.

TABLE 2
WNT Agonists Sequences

| WNT | SEQ ID | |
|---|---|---|
| AGONIST | NO: | SEQUENCE |
| R2M3- | 1 | EVQLVQSGAEVKKPGASVKVSCKASGYTFTSYGISWVRQAPGQG |
| 26HC | | LEWMGWISAYNGNTNYAQKLQGRVTMTTDTSTSTAYMELRSLRS |
| | | DDTAVYYCASSKEKATYYYGMDVWGQGTTVTVSSASTKGPSVFP |
| | | LAPSSKSTSGGTAALGCLVKDYFPEPVTVSWNSGALTSGVHTFPA |
| | | VLQSSGLYSLSSVVTVPSSSLGTQTYICNVNHKPSNTKVDKKVEPK |
| | | SCDKTHTCPPCPAPEAAGGPSVFLFPPKPKDTLMISRTPEVTCVVV |
| | | DVSHEDPEVKFNWYVDGVEVHNAKTKPREEQYNSTYRVVSVLTV |
| | | LHQDWLNGKEYKCKVSNKALGAPIEKTISKAKGQPREPQVYTLPP |
| | | SREEMTKNQVSLTCLVKGFYPSDIAVEWESNGQPENNYKTTPPVL |
| | | DSDGSFFLYSKLTVDKSRWQQGNVFSCSVMHEALHNHYTQKSLS |
| | | LSPGK |
| R2M3-26 | 2 | DVQLVESGGGLVQAGGSLRLACAGSGRIFAIYDIAWYRHPPGNQR |
| LC | | ELVAMIRPVVTEIDYADSVKGRFTISRNNAMKTVYLQMNNLKPED |
| | | TAVYYCNAKRPWGSRDEYWGQGTQVTVSSGSGSGQAVVLQEPS |
| | | LSVSPGGTVTLTCGLSSGSVSTNYYPSWYQQTPGQAPRTLIYYTNT |
| | | RSSDVPERFSGSIVGNKAALTITGAQPDDESVYFCLLYLGRGIWVF |
| | | GGGTKLTVLGQPKAAPSVTLFPPSSEELQANKATLVCLISDFYPGA |
| | | VTVAWKADSSPVKAGVETTTPSKQSNNKYAASSYLSLTPEQWKS |
| | | HRSYSCQVTHEGSTVEKTVAPTECS |
| R2M13-03 | 3 | EVQLLQSGAEVKKPGSSVKVSCKASGGTFTYRYLHWVRQAPGQG |
| HC | | LEWMGGIIPIFGTGNYAQKFQGRVTITADESTSTAYMELSSLRSED |
| | | TAVYYCASSMVRVPYYYGMDVWGQGTLVTVSSASTKGPSVFPLA |
| | | PSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSC |
| | | DKTHTCPPCPAPEAAGGPSVFLFPPKPKDTLMISRTPEVTCVVVDV |
| | | SHEDPEVKFNWYVDGVEVHNAKTKPREEQYNSTYRVVSVLTVLH |
| | | QDWLNGKEYKCKVSNKALGAPIEKTISKAKGQPREPQVYTLPPSR |
| | | EEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSP |
| | | GK |
| R2M13-03 | 4 | DVQLVESGGGLVQPGGSLRLSCTSSANINSIETLGWYRQAPG |
| LC | | KQRELIANMRGGGYMKYAGSLKGRFTMSTESAKNTMYLQ |
| | | MNSLKPEDTAVYYCYVKLRDDDYVYRGQGTQVTVSSGGS |
| | | GSGSGDIQMTQSPSSLSASVGDRVTITCRASQSISSYLNWYQ |
| | | QKPGKAPKLLIYAASSLQSGVPSRFSGSGSGTDFTLTISSLQP |
| | | EDFATYYCQQSYSTPLTFGGGTKVEIKRTVAAPSVFIFPPSDE |
| | | QLKSGTASVVCLLNNFYPREAKVQWKVDNALQSGNSQESV |
| | | TEQDSKDSTYSLSSTLTLSKADYEKHKVYACEVTHQGLSSP |
| | | VTKSFNRGEC |
| 1RC07-03 | 5 | QVQLQQWGAGLLKPSETLSLTCAVSGASFSGHYWTWIRQPP |
| HC | | GKGLEWIGEIDHTGSTNYEPSLRSRVTISVDTSKNQFSLNLKS |
| | | VTAADTAVYYCARGGQGGYDWGHYHGLDVWGQGTTVTV |
| | | SSASTKGPSVFPLAPSSKSTSGGTAALGCLVKDYFPEPVTVS |
| | | WNSGALTSGVHTFPAVLQSSGLYSLSSVVTVPSSSLGTQTYI |
| | | CNVNHKPSNTKVDKKVEPKSCDKTHTCPPCPAPEAAGGPSV |
| | | FLEPPKPKDTLMISRTPEVTCVVVDVSHEDPEVKFNWYVDG |
| | | VEVHNAKTKPREEQYNSTYRVVSVLTVLHQDWLNGKEYKC |
| | | KVSNKALGAPIEKTISKAKGQPREPQVYTLPPSREEMTKNQV |
| | | SLTCLVKGFYPSDIAVEWESNGQPENNYKTTPPVLDSDGSFF |
| | | LYSKLTVDKSRWQQGNVFSCSVMHEALHNHYTQKSLSLSP |
| | | GK |
| 1RC07-03 | 6 | DVQLVESGGGLVQPGGSLRLSCTSSANINSIETLGWYRQAPG |
| LC | | KQRELIANMRGGGYMKYAGSLKGRFTMSTESAKNTMYLQ |
| | | MNSLKPEDTAVYYCYVKLRDDDYVYRGQGTQVTVSSGSGS |
| | | GSYVLTQPPSVSVSPGQTASITCSGDKVGHKYASWYQQKPG |
| | | QSPVLVIYEDSQRPSGIPVRFSGSNSGNTATLTISGTQAMDEA |
| | | DYYCQAWDSSTDVVFGGGTKLTVLGQPKAAPSVTLFPPSSE |
| | | ELQANKATLVCLISDFYPGAVTVAWKADSSPVKAGVETTTP |
| | | SKQSNNKYAASSYLSLTPEQWKSHRSYSCQVTHEGSTVEKT |
| | | VAPTECS |

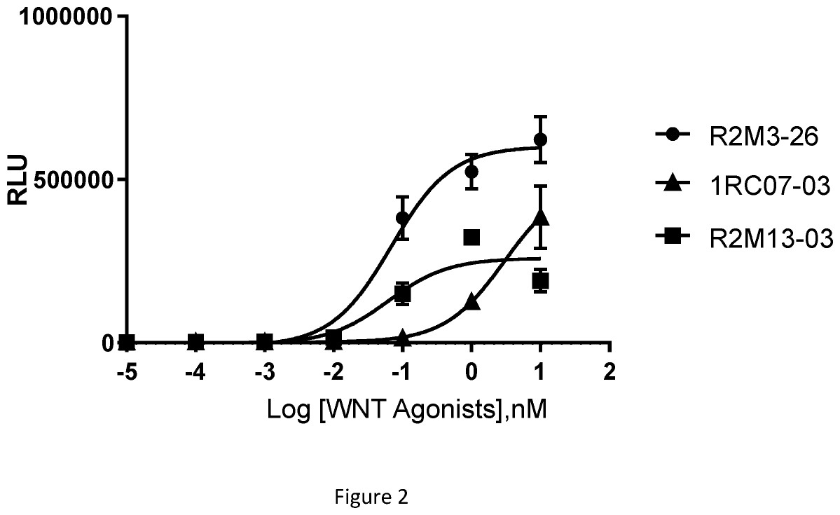

Cells were seeded at 1 million per 96-well plate on Day 1 and grown overnight. Proteins were added to the cells in 10-fold dilutions starting from 100 nM in triplicates in the presence of 20 nM R-spondin. Luciferase reporter activities were assayed in the wells with Luciferase Assay System (Promega) and read on a SpectraMax plate reader (Molecular Devices). Mean absolute RLU values of the triplicates for each protein dilution are shown (FIG. 2). R2M3-26 (FZD1, 2, 5, 7, 8) showed highest reporter activity. R2M13-03 (FZD5,8) showed the lowest activity in the STF assay.

### IV. Activation of WNT Signaling Specifically Through FZD5 and FZD8 Stimulated Mouse Small Intestine Organoid Proliferation

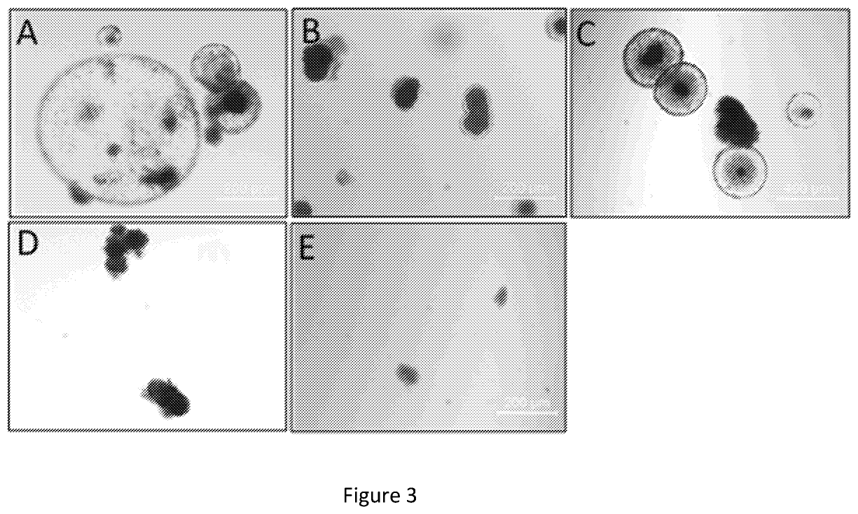

Mouse small intestinal organoids were maintained in mouse IntestiCult™ Organoid Growth Medium (StemCell Technologies). To assay for organoid proliferation, organoids were dissociated with Gentle Cell Dissociation Reagent (StemCell Technologies) for 10 min with shaking, washed twice in cold PBS (Gibco) and resuspended 1:1 in Matrigel (Corning) on ice. 25 μl of cell resuspension in Matrigel was seeded to the center of each well on a prewarmed 48-well tissue culture plate and let solidify for 5 min at 37° C. 300 μl of Basal Media (Table 3), Basal Media+IWP2 or Basal Media+IWP2+surrogate WNT agonist was applied to the wells. Each condition included 5-6 repeats. Media and treatments were changed once on Day 4 after plating. Images of the 3D cultured organoids acquired on Day 7 are shown in FIG. 3. Cell Titer Glow 3D (Promega) was performed on the treated organoids on Day 7.

TABLE 3
Basal Media composition

| | | |
|---|---|---|
| DMEM/F12K | Life technologies | |
| HEPES | Life technologies | 10 mM |
| Penicillin/streptomycin | Life technologies | 1X |
| GlutaMAX | Life technologies | 1X |
| N2 supplement 100× | Life technologies | 1X |
| B27 Supplement 50× | Life technologies | 1X |
| N-acetylcysteine | Sigma-Aldrich | 1.25 mM |
| Recombinant human EGF | Peprotech | 50 ng/mL |
| Recombinant human Noggin | Peprotech | 50 ng/mL |
| Recombinant human Rspondin-1 | R&D Systems | 500 ng/mL |

Intestinal organoids proliferate and became morphologically round in the presence of WNT agonist treatment. Endogenous WNT expression was inhibited in the assay with the Porcupine inhibitor IWP-2. Both R2M3-26 (FZD1, 2, 5, 7, 8) and R2M13-03 (FZD5,8) potently stimulated organoid proliferation, reflected by increasing numbers of organoids and enlargement of individual organoid (FIGS. 3A and 3C). 1RC07-03 (FZD1, 2, 7) also stimulated organoid proliferation but to a much lesser extent. All WNT agonists demonstrated higher activities than 18R5-Dkk1c (whose structure is described in Janda et al. (2017) Nature 545:234-237). FZD antagonists can be tested similarly in organoid cultures.

### VI. IHC Analysis of Mouse Organoid Cultures

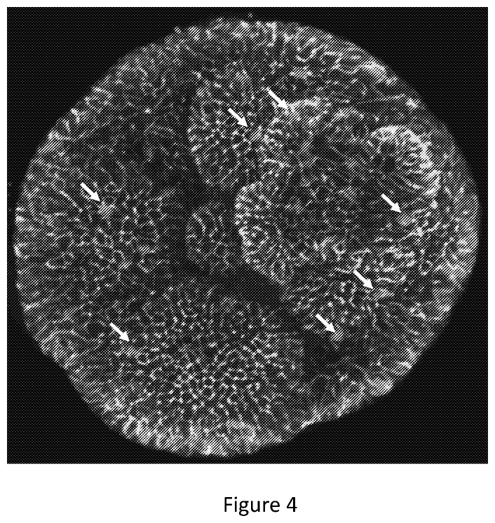

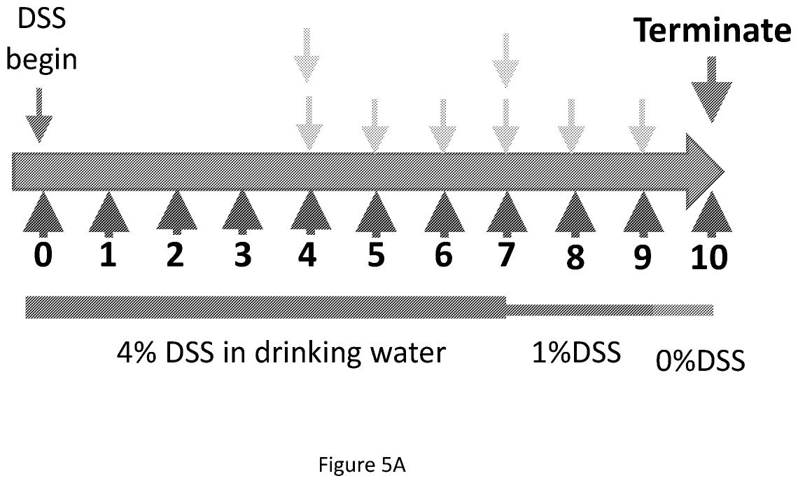

Activities of R2M3-26 on mouse small intestine organoids were demonstrated with the proliferation marker, Ki67, stain. Mouse small intestinal organoids grown in media submerged Matrigel in an 8-well chamber slide (Lab-Tek II, 154534) were treated with 100 nM R2M3-26 as describe above for 7 days. Organoids were then fixed in 4% PFA, permeabilized in PBS+0.2% Triton for 20 min and blocked in Blocking Buffer (PBS+0.2% Triton+3% BSA). Primary antibodies rabbit anti-Ki67 (Abeam ab15580, 1:1000) and goat anti-E-cadherin (R&D AF748, 1:2000) were mixed in Blocking Buffer and added to organoids. After 1 hour incubation with primary antibody at room temperature, organoids were washed with 3 times PBS+0.2% Triton before incubating with 1:1000 dilution of secondary antibodies donkey anti-rabbit Alexa568 (Abeam) and donkey anti-goat Alexa488 (Abeam) for 30 min at room temperature. Organoids were then washed 3 times with PBS+0.1% Tween and mounted in ProLong™ Gold Antifade Mountant (Thermo Fisher). Z-stack signal channel images were taken with a Zeiss DMi8 fluorescence microscope, digitally deconvoluted, projected on 2D and the two channels merged for illustration. WNT agonist treatment stimulated proliferation of mouse small intestine organoids. Mouse small intestinal organoid after treating with 100 nM R2M3-26 stained with anti-Ki67 (red) and anti-E-Cadherin (green) showing cell proliferation upon WNT agonist treatment (FIG. 4).

### VII. In Vivo Dextran Sulfate Sodium (“DSS”) IBD Mouse Model

Six-week old C57Bl/6J female mice were obtained from Jackson Laboratories (Bar Harbor, Me., USA) and were housed 4 per cage. All animal experimentation was in accordance with the criteria of the “Guide for the Care and Use of Laboratory Animals” prepared by the National Academy of Sciences. Protocols for animal experimentation were approved by the Surrozen Institutional Animal Care and Use Committee. Mice were acclimatized a minimum of two days prior to initiating experiments. Mice were kept 12/12-hour light/dark cycle in a 30% to 70% humidity environment and room temperature ranging from 20° C. to 26° C.

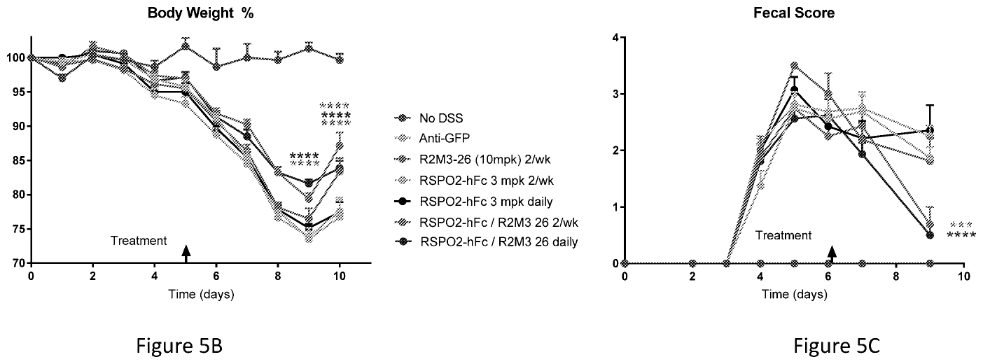

To induce acute colitis, 7- to 8-week-old female mice were given drinking water containing 4.0% (w/v) Dextran Sulfate Sodium (DSS, MP Biomedicals, MFCD00081551) ad libitum for 7 days and drinking water containing 1.0% (w/v) DSS for 2 days (FIG. 5A). Mice subjected to DSS developed severe colitis characterized by profound and sustained weight loss (FIG. 5B) and bloody diarrhea, resulting in the increase of the disease index (FIG. 5C) as represented by fecal score. The RSPO2-Fc (R-Spondin 2-Fc; SEQ ID NO: 24) plus R2M3-26 combination treatment, twice weekly or daily, significantly improved disease activity index (DAI) at day 9 compared to negative controls. R2M3-26 alone and R2M3-26 plus RSPO2-Fc treatments significantly improved body weight at day 10.

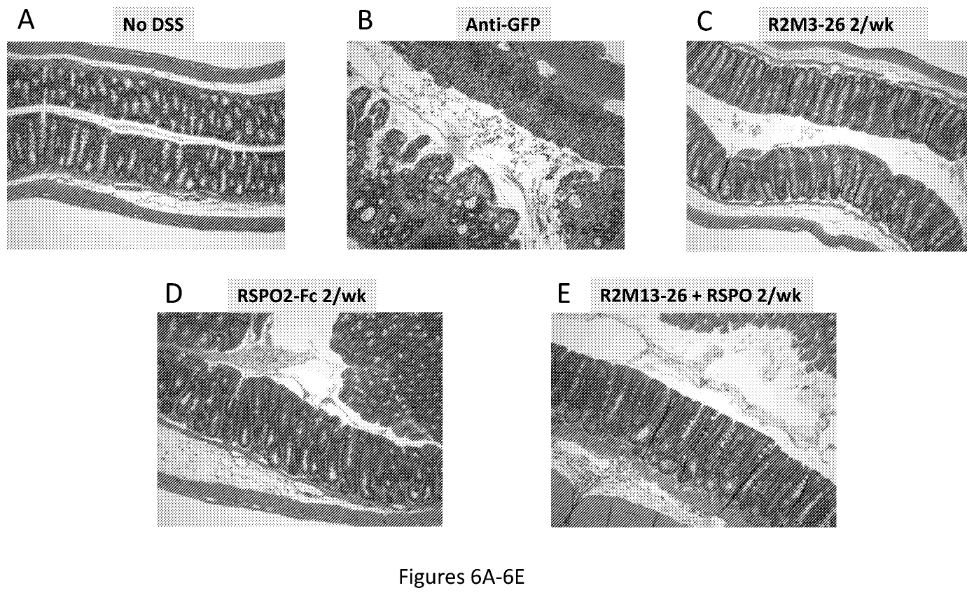

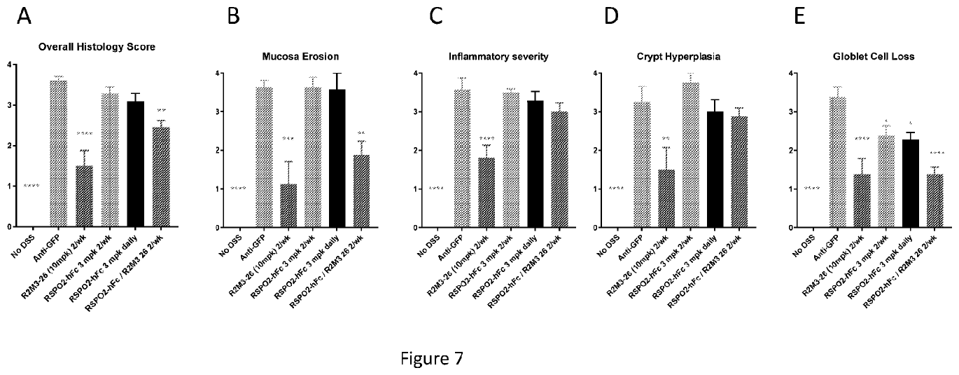

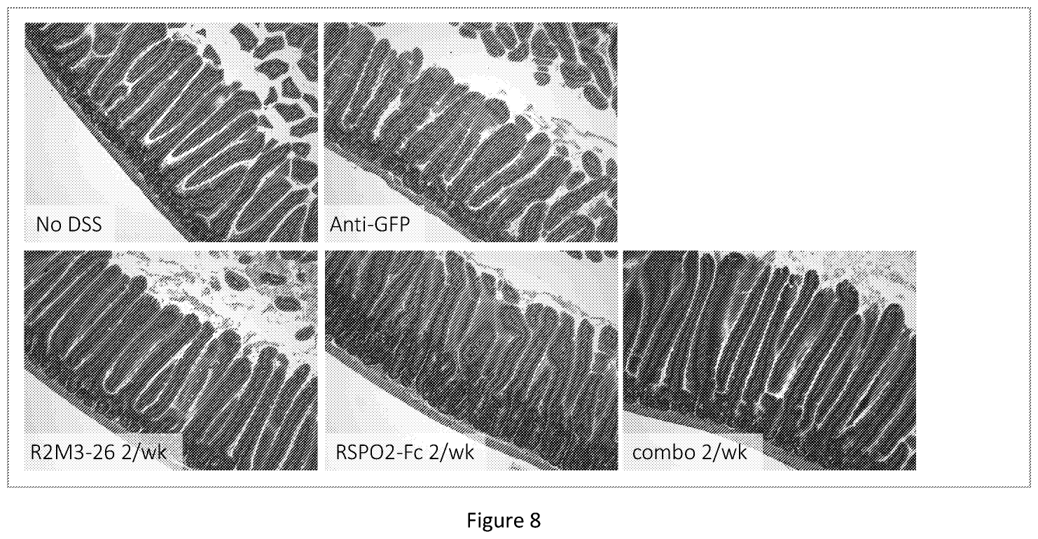

Histological evaluation of the transverse colon of DSS model mice showed inflammation extending from the mucosa to the serosa, crypt hyperplasia, goblet cell loss and ulceration (FIGS. 6A-6E); in contrast, the colon of WNT agonist-treated mice were almost normal, with the lowest histological score among all the treatment groups (FIG. 6C). RSPO2-Fc has no significant positive effect on histology score of colon tissue. The RSPO2-Fc plus R2M3-26 combo group has lower histology score of colon tissue compared with the control anti-GFP group (P<0.0, FIG. 7A). R2M3-26 did not appear to affect small intestine crypts or villi (FIG. 8C), while RSPO2-Fc and combo induced hyperplasia of villi and crypts (FIGS. 8D-8E). Histology scoring was assessed as described in Geboes, et al. (2000) supra.

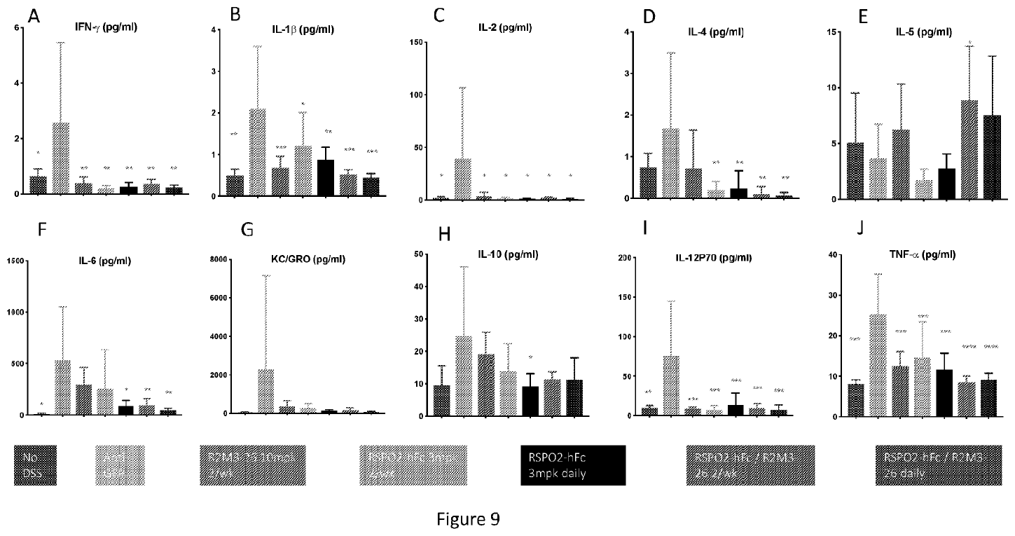

The serum inflammatory cytokines were analyzed by Proinflammatory Panel 1 kits (Meso Scale Diagnostics, K15048D), and the treatment of R2M3-26, RSPO2-Fc, and R2M3-26 plus RSPO2-Fc, all reduce the cytokine levels of IFN-γ, TNF-α, and IL-1β (FIGS. 9A-9J, specifically FIGS. 9A, 9J, and 9B, respectively).

RSPO2-Fc alone induced small intestine hyperplasia and had no significant benefit on body weight loss and DAI. The WNT agonist/RSPO2-Fc combo treatment reduced disease activity, repaired damaged colon epithelium, while induced hyperplasia in small intestine. R2M3-26 alone: a) improved body weight; b) repaired damaged colon epithelium; c) decreased serum inflammatory cytokine markers; and d) did not cause small intestine hyperplasia, thus demonstrating that the WNT agonist alone can be used to treat acute colon colitis by improving the epithelial barrier thereby reducing inflammation.

### VIII. Improvement of Intestinal Inflammation and Epithelial Tissue Repair

The previous study demonstrated that polyspecific WNT agonist, R2M3-26, was able to improve intestinal inflammation and repair epithelial damage in DSS colitis mouse model. Given the selective expression of FZD 5 and FZD 8 in the colon, a FZD5, 8 specific WNT agonist, R2M13-26, and FZD1, 2, 7 specific WNT agonist, 1RC07-26, were tested to ascertain if either or both were able to mitigate DSS induced colitis in a mouse model.

Six-week old C57Bl/6J female mice were obtained from Jackson Laboratories (Bar Harbor, Me., USA) and were housed 5 per cage. All animal experimentation was in accordance with the criteria of the “Guide for the Care and Use of Laboratory Animals” prepared by the National Academy of Sciences. Protocols for animal experimentation and were approved by the internal Institutional Animal Care and Use Committee.

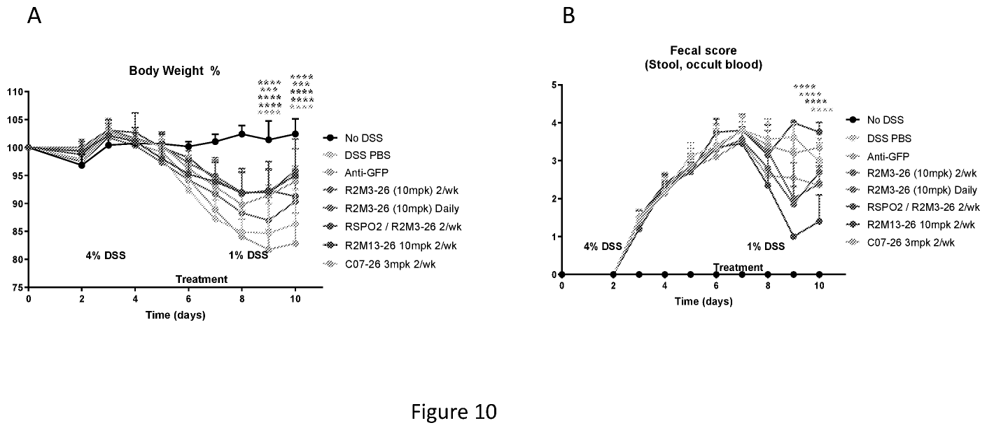

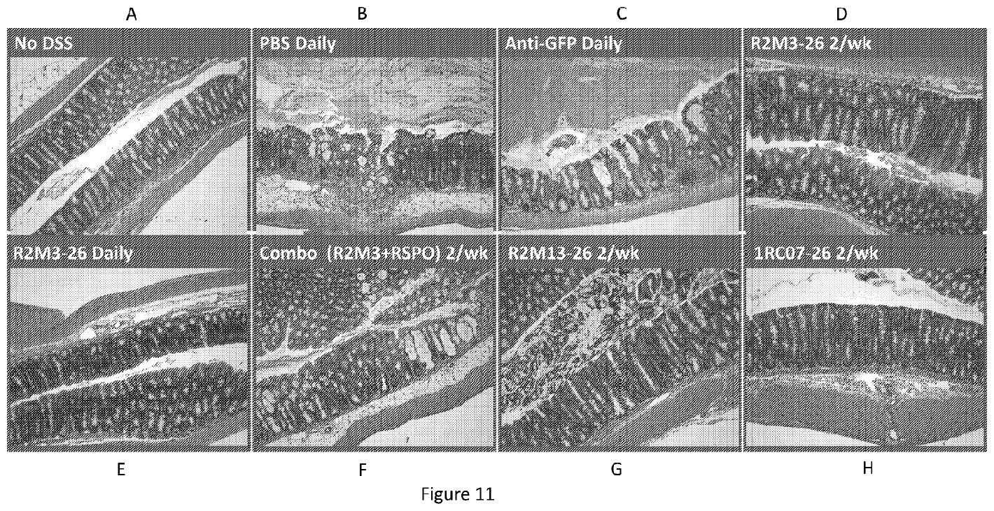

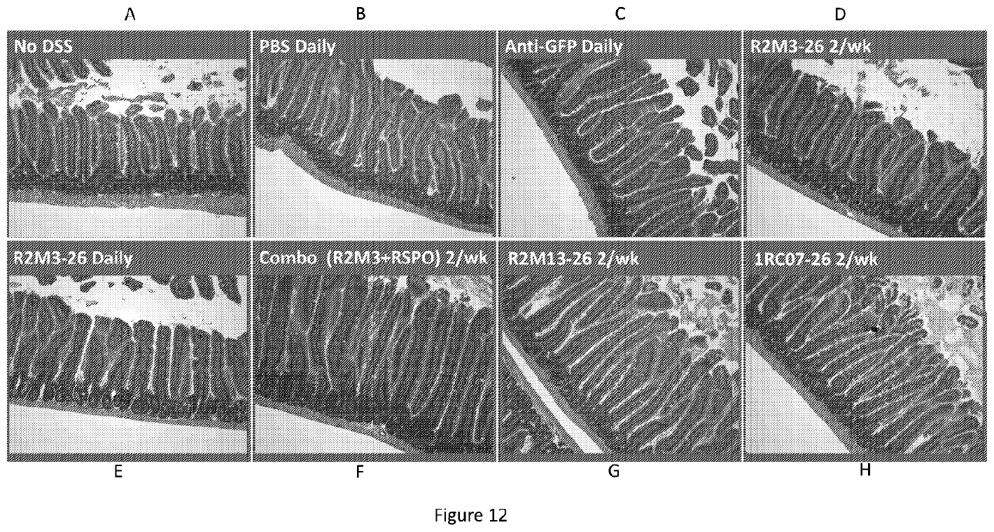

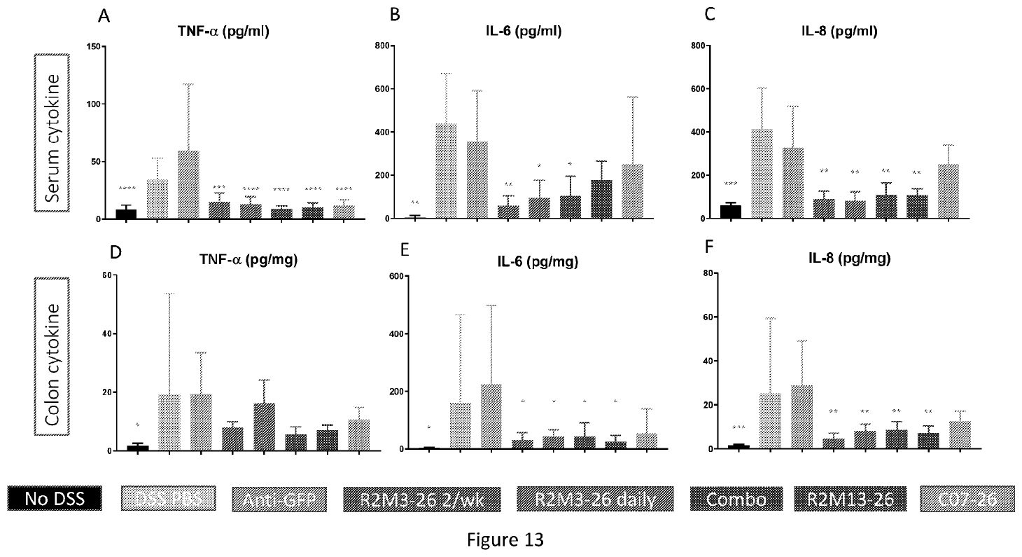

To induce acute colitis, 7- to 8-week-old female mice were given drinking water containing 4.0% (w/v) Dextran Sulfate Sodium (DSS, MP Biomedicals, MFCD00081551) ad libitum for 7 days and drinking water containing 1.0% (w/v) DSS for 3 days. Mice subjected to DSS developed severe colitis characterized by profound and sustained weight loss (FIG. 10A) and bloody diarrhea, resulting in the increase of the fecal score (FIG. 10B) and disease activity index (DAI). The R2M3-26, R2M13-26, and 1RC07-26, respectively, treatments, twice weekly, significantly improved body weight (FIG. 10A) and fecal score (FIG. 10B) at day 10 compared to negative controls (PBS or Anti-GFP). Histological evaluation of the transverse colon of DSS model mice showed neutrophils infiltration, crypt hyperplasia, goblet cell loss and ulceration (FIGS. 11B and 11C). The R2M3-26, R2M13-26, and 1RC07-26 treatments repaired colon histology, showing improvement on epithelial erosion, goblet cell loss and neutrophils migration (FIGS. 11D-H). R2M3-26, R2M13-26, or C07-3 did not cause small Intestine hyperplasia (FIGS. 11B and 11C), while R2M3-26/RSPO Combo treatment induces small Intestine hyperplasia (FIG. 12D-H). The inflammatory cytokines in the serum and colon tissue were analyzed using a Proinflammatory Panel 1 kits (Meso Scale Diagnostics, K15048D), and the results indicated that R2M3-26 and R2M13-26 treatment significantly reduced TNF-α and IL-8 level in the serum (FIGS. 13A and 13C), and IL-6 and IL-8 level in the colon tissue (FIGS. 13E and 13F).

As noted in Example IV above, R2M3-26 reduced intestinal inflammation and repaired epithelial damage in DSS colitis mouse. This study further demonstrated that FZD5, 8 specific WNT agonist, R2M13-26, and FZD1, 2, 7 specific WNT agonist, 1RC07-26, were able to improve DAI, repair damaged colon epithelium without small intestine hyperplasia, and reduce inflammatory cytokine levels in colon and serum.

### IX. Dose Response Analysis of R2M13-26 in Mouse DSS Model

To determine the optimum dose of R2M13-26 (FZD5, 8 binder) in the DSS mouse model of IBD, six-week old C57Bl/6J female mice were obtained from Jackson Laboratories (Bar Harbor, Me., USA) and were housed 5 per cage. All animal experimentation was in accordance with the criteria of the “Guide for the Care and Use of Laboratory Animals” prepared by the National Academy of Sciences. Protocols for animal experimentation were approved by the Surrozen Institutional Animal Care and Use Committee.

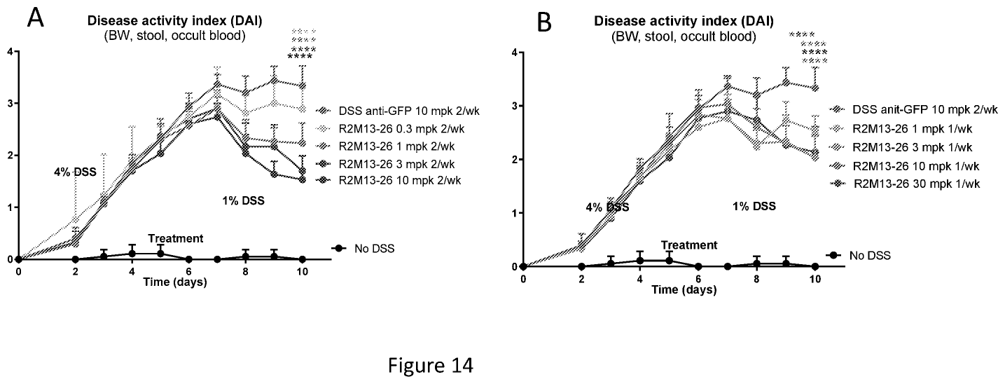

To induce acute colitis, 7- to 8-week-old female mice were given drinking water containing 4.0% (w/v) Dextran Sulfate Sodium (DSS, MP Biomedicals, MFCD00081551) ad libitum for 7 days and drinking water containing 1.0% (w/v) DSS for 3 days. Control mice subjected to DSS developed severe colitis characterized by profound and sustained weight loss and bloody diarrhea, resulting in the increase of disease activity index (DAI, FIGS. 14A-14B).

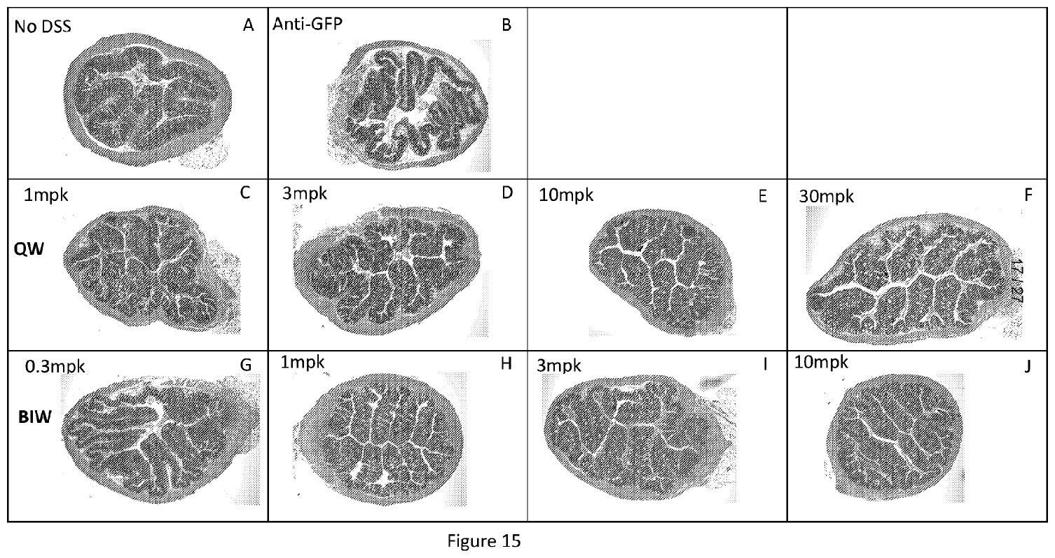

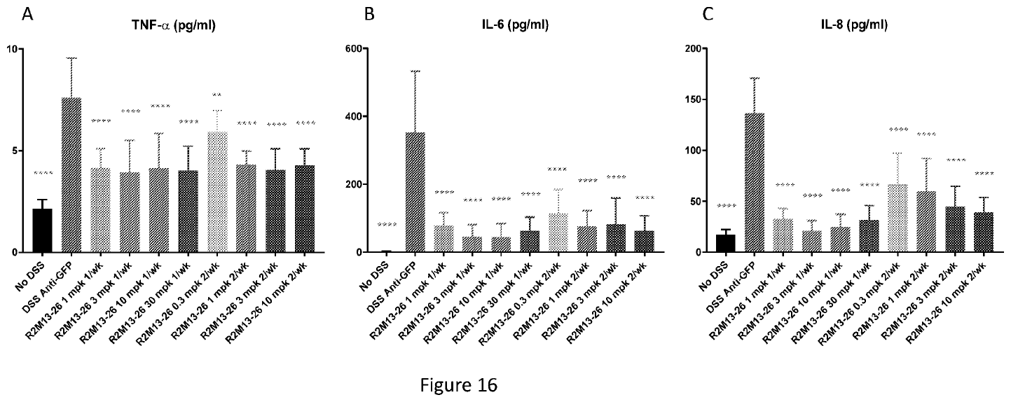

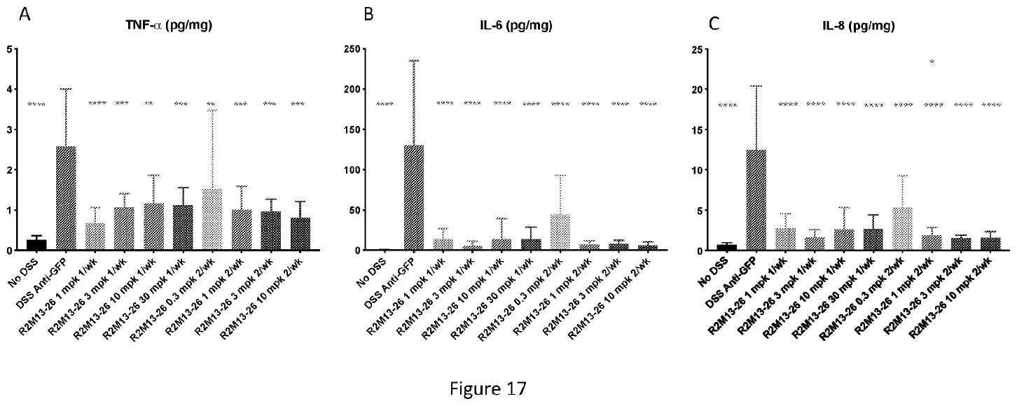

R2M13-26 treatment at 0.3, 1, 3, 10 mpk, twice weekly, improved DAI with a dose response pattern (FIG. 14A). R2M13-26 treatment at further concentrations of 1, 3, 10, 30 mpk, once weekly, improved DAI with a dose response pattern (FIG. 14B). Histological evaluation of the cross sections of transverse colon of DSS model mice showed neutrophils infiltration, edema, crypt hyperplasia, goblet cell loss and ulceration. The R2M13-26 treatments, with different dose and frequency, all showed improvement on epithelial erosion, goblet cell loss and neutrophils migration in the DSS colitis mice (FIGS. 15A-15J). The inflammatory cytokines in the serum and colon tissue were analyzed by Proinflammatory Panel 1 kits (Meso Scale Diagnostics, K15048D), and the results indicated that R2M13-26 treatment, with different dose and frequency, all significantly decreased TNF-α, IL-6 and IL-8 level in serum FIGS. 16A-16C) and in the colon tissue (FIG. 17A-17C).

R2M13-26, with a wide dose range, reduced intestinal inflammation and repaired epithelial damage in DSS colitis mouse model, further validating the FZD5, 8 specific molecule (R2M13-26) in treating acute colitis through improvement of the intestinal epithelial barrier.

### X. Efficacy of Different FZD5,8-Specific WNT Agonists in the Acute DSS Model

Activities of four FZD5,8-specific WNT agonists, 57SE8-26, 57SB8-26, 174R-E01-26 and 57SA10-26 were examined in the human hepatic cell line, Huh7 cells to determine their ability to activate WNT signaling. Table 4 provides the sequences of components of the FZD5, 8 WNT agonists. These WNT agonists comprise a FZD binding domain that is a Fab containing a Heavy Chain (HC) and Light Chain (LC) and a LRP binding domain that is a VHH attached to the FZD Fab at the N-terminus of the LC via a linker. The WNT agonists include two of the indicated LC chains and two of the indicated HC chains.

TABLE 4
FZD5,8 Specific WNT Agonists

| | SEQ | |
|---|---|---|
| WNT | ID | |
| AGONIST | NO: | SEQUENCE |
| 57SE8-26 | 7 | EVQLVQSGAEVKKPGASVKVSCKASGYTFTNYYMHWVRQA |
| HC | | PGQGLEWMGWINPNSGGTNYAQKFQGRVTMTRDTSTSTVY |
| | | MELSSLRSEDTAVYYCARGHWYFDLWGRGTLVTVSSASTKGP |
| | | SVFPLAPSSKSTSGGTAALGCLVKDYFPEPVTVSWNSGALTSGVHTFP |
| | | 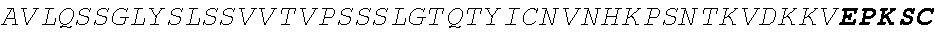 |
| | | 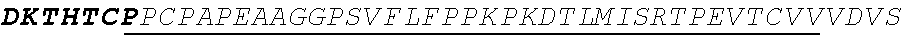 |
| | | HEDPEVKFNWYVDGVEVHNAKTKPREEQYNSTYRVVSVLTVLHQDW |
| | | LNGKEYKCKVSNKALGAPIEKTISKAKGQPREPQVYTLPPSREEM |
| | | TKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGK |
| 57SE8-26 LC | 8 | 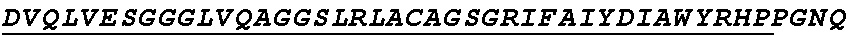 |
| | | 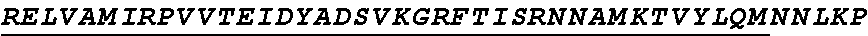 |
| | | 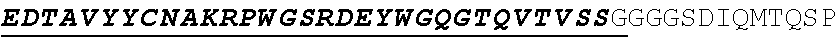 |
| | | 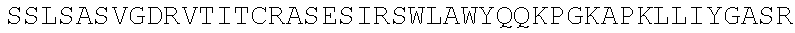 |
| | | 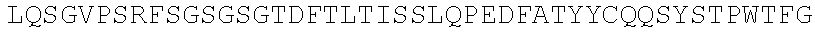 |
| | | 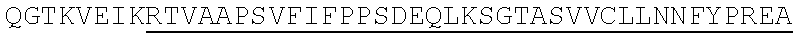 |
| | | 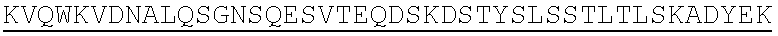 |
| | | 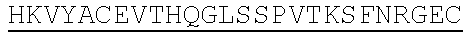 |
| 57SB8-26 | 9 | EVQLVQSGAEVKKPGSSVKVSCKASGYTFTKDYMHWVRQAP |
| HC | | GQGLEWMGGIIPIFGTANYAQRFQGRVTITADESTSTAYMEL |
| | | SSLRSEDTAVYYCARGLPPAAGGGGYFQHWGQGTLVTVSSAS |
| | | TKGPSVFPLAPSSKSTSGGTAALGCLVKDYFPEPVTVSWNSGALTSGV |
| | | 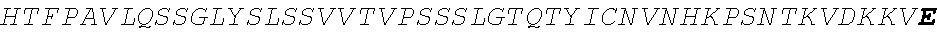 |
| | | 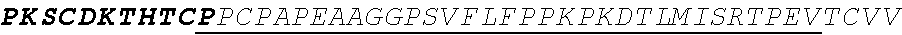 |
| | | VDVSHEDPEVKFNWYVDGVEVHNAKTKPREEQYNSTYRVVSVLTVLH |
| | | QDWLNGKEYKCKVSNKALGAPIEKTISKAKGQPREPQVYTLPPSR |
| | | EEMTKNQVSLTCLVKGFYPSDIAVEWESNGQPENNYKTTPPV |
| | | LDSDGSFFLYSKLTVDKSRWQQGNVFSCSVMHEALHNHYTQ |
| | | KSLSLSPGK |
| 57SB8-26 | 10 | 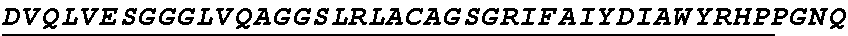 |
| LC | | 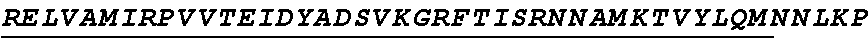 |
| | | 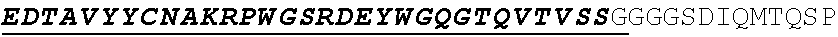 |
| | | 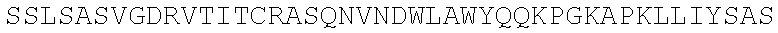 |
| | | 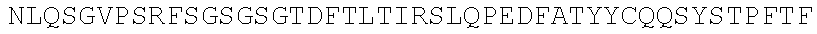 |
| | | 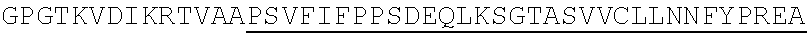 |
| | | 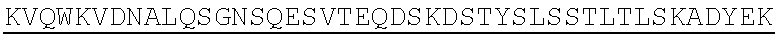 |
| | | 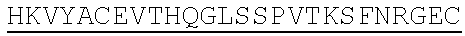 |
| 174R-E01-26 | 11 | EVQLVQSGGGVVQPGRSLRLSCAASGFTFSSYGMHWVRQAP |
| HC | | GKGLEWVAVISYDGSNKYYADSVKGRFTISRDNSKNTLYLQM |
| | | NSLRAEDTAVYYCAGDTFGVGHFYWGQGTLVTVSSASTKGPS |
| | | VFPLAPSSKSTSGGTAALGCLVKDYFPEPVTVSWNSGALTSGVHTFPA |
| | | 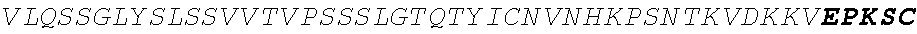 |
| | | 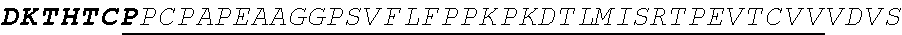 |
| | | HEDPEVKFNWYVDGVEVHNAKTKPREEQYNSTYRVVSVLTVLHQDW |
| | | LNGKEYKCKVSNKALGAPIEKTISKAKGQPREPQVYTLPPSREEM |
| | | TKNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGK |
| 174R-E01-26 | 12 | 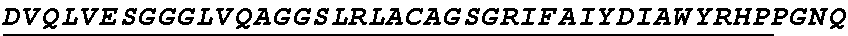 |
| HC | | 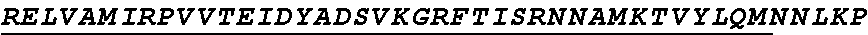 |
| | | 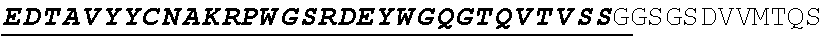 |
| | | 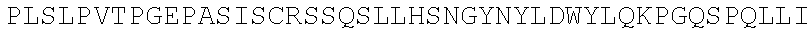 |
| | | 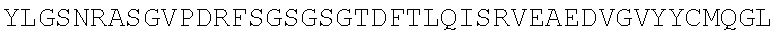 |
| | | 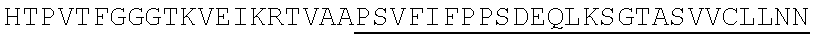 |
| | | 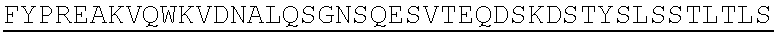 |
| | | 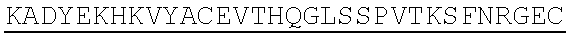 |
| 57SA10-26 | 13 | EVQLVQSGAEVKKPGSSVKVSCKASGGTFSSSVISWVRQAPG |
| HC | | QGLEWMGWISVYNGNTNYAEKFQGRVTITADESTSTAYMEL |
| | | SSLRSEDTAVYYCARFAMVRGGVYYFDYWGQGTLVTVSSAST |
| | | KGPSVFPLAPSSKSTSGGTAALGCLVKDYFPEPVTVSWNSGALTSGVH |
| | | 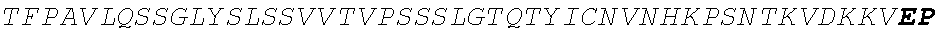 |
| | | 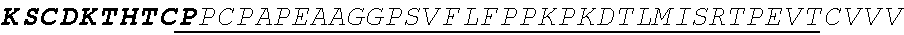 |
| | | DVSHEDPEVKFNWYVDGVEVHNAKTKPREEQYNSTYRVVSVLTVLH |
| | | QDWLNGKEYKCKVSNKALGAPIEKTISKAKGQPREPQVYTLPPSR |
| | | EEMTKNQVSLTCLVKGFYPSDIAVEWESNGQPENNYKTTPPV |
| | | LDSDGSFFLYSKLTVDKSRWQQGNVFSCSVMHEALHNHYTQ |
| | | KSLSLSPGK |
| 57SA10-26 | 14 | 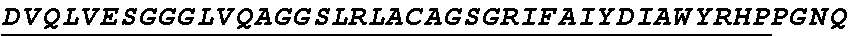 |
| LC | | 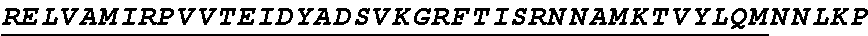 |
| | | 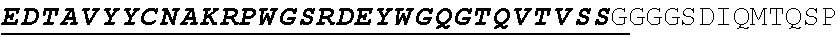 |
| | | 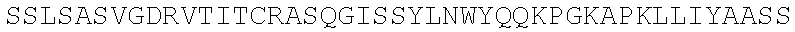 |
| | | 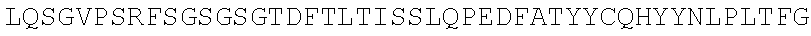 |
| | | 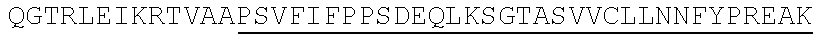 |
| | | 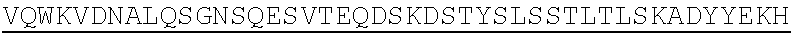 |
| | | 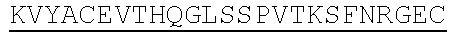 |

The FZD-VH sequence is indicated in bold; the FZD-CH1 sequence is indicated in italics; the hinge sequence is indicated in bold italics; the CH2 sequence is indicated in underlined italics; the CH3 sequence is indicated in bold underline; the LRP (26) VHH sequence, which is attached to the N-terminus of the VL via a linker, is indicated in bold italic underline; the linker sequence is underline only; the FZD-VL is shaded gray; and the FZD-CL is shaded gray and underlined.

FZD5/8 specific binding domains that specifically bind FZD5 and FZD8 (and do not significantly bind other FZDs) are shown in Table 5.

TABLE 5
FZD5 8 Specific Binding Domains

| FZD5/8 | SEQ | |
|---|---|---|
| Binding | ID | |
| Domain | NO: | SEQUENCE |
| 57SE8 | 25 | CAGGTGCAGCTGGTGCAGTC |
| VH | | TGGGGCTGAGGTGAAGAAGC |
| (poly | | CTGGGGCNTCAGTGAAGGTT |
| nucleotide) | | TCNTGCAAGGCATCTGGATA |
| | | CACNTTCACCAACTACTATA |
| | | TGCACTGGGTGCGTCAGGCC |
| | | CCTGGACAAGGGCTTGAGTG |
| | | GATGGGATGGATCAACCCTA |
| | | ACAGTGGTGGCACAAATTAT |
| | | GCACAGAAGTTTCAGGGCCG |
| | | TGTCACCATGACCCGCGACA |
| | | CGTCCACGAGCACAGTCTAC |
| | | ATGGAGCTGAGCAGCCTGCG |
| | | TTCTGAGGACACGGCCGTGT |
| | | ATTACTGTGCGAGAGGGCAC |
| | | TGGTACTTCGATCTCTGGGG |
| | | CCGTGGCACCCTGGTCACCG |
| | | TCTCCTCA |
| 57SE8 | 26 | GACATCCGGATGACCCAGTC |
| VL | | TCCATCCTCCCTGTCTGCAT |
| (poly | | CTGTAGGAGACAGAGTCACC |
| nucleotide) | | ATCACTTGCCGGGCCAGTGA |
| | | GAGTATTAGGAGCTGGTTGG |
| | | CCTGGTATCAGCAGAAACCA |
| | | GGGAAAGCCCCTAAGCTCCT |
| | | GATCTATGGTGCATCGCGTT |
| | | TGCAAAGTGGGGTCCCATCA |
| | | AGGTTCAGTGGCAGTGGATC |
| | | TGGGACAGATTTCACTCTCA |
| | | CCATCAGCAGTCTGCAACCT |
| | | GAAGATTTTGCAACTTACTA |
| | | CTGTCAACAGAGTTACAGTA |
| | | CCCCTTGGACGTTCGGCCAA |
| | | GGTACCAAGGTGGAAATCAA |
| | | A |
| 57SB8 | 27 | GAGGTGCAGCTGGTGCAGTC |
| VH | | TGGGGCTGAGGTGAAGAAGC |
| (poly | | CTGGGTCCTCGGTGAAGGTC |
| nucleotide) | | TCCTGCAAGGCTTCTGGATA |
| | | CACCTTCACCAAAGACTATA |
| | | TGCACTGGGTGCGGCAGGCC |
| | | CCTGGACAAGGGCTTGAGTG |
| | | GATGGGAGGGATCATCCCTA |
| | | TATTTGGTACAGCAAACTAC |
| | | GCACAGAGGTTCCAGGGCCG |
| | | GGTCACGATTACCGCGGACG |
| | | AATCCACGAGCACAGCCTAC |
| | | ATGGAGCTGAGCAGCCTGCG |
| | | GTCTGAGGACACGGCCGTGT |
| | | ATTACTGTGCGAGAGGACTC |
| | | CCACCAGCAGCTGGTGGCGG |
| | | CGGATACTTCCAGCACTGGG |
| | | GCCAGGGCACCCTGGTCACC |
| | | GTCTCCTCA |
| 57SB8 | 28 | GACATCCAGATGACCCAGTC |
| VL | | TCCATCCTCCCTGTCTGCAT |
| (poly | | CTGTAGGAGACAGAGTCACC |
| nucleotide) | | ATCACTTGCCGGGCCAGTCA |
| | | GAATGTTAATGACTGGTTGG |
| | | CCTGGTATCAGCAGAAACCA |
| | | GGGAAAGCCCCTAAGCTCCT |
| | | GATCTATAGTGCATCCAATT |
| | | TGCAATCTGGGGTCCCATCA |
| | | AGGTTCAGTGGCAGTGGATC |
| | | TGGGACAGATTTCACTCTCA |
| | | CCATCCGCAGTCTGCAACCT |
| | | GAAGATTTTGCAACTTACTA |
| | | CTGTCAACAGAGCTACAGTA |
| | | CCCCATTCACTTTCGGCCCT |
| | | GGTACCAAAGTGGATATCAA |
| | | A |
| 174R-E01 | 29 | GAGGTCCAGCTGGTGCAGTC |
| VH | | TGGGGGAGGCGTGGTCCAGC |
| (poly | | CTGGGAGGTCCCTGAGACTC |
| nucleotide) | | TCCTGTGCAGCCTCTGGATT |
| | | CACCTTCAGTAGCTATGGCA |
| | | TGCACTGGGTCCGCCAGGCT |
| | | CCAGGCAAGGGGCTGGAGTG |
| | | GGTGGCAGTTATATCATATG |
| | | ATGGAAGTAATAAATACTAT |
| | | GCAGACTCCGTGAAGGGCCG |
| | | ATTCACCATCTCCAGAGACA |
| | | ATTCCAAGAACACGCTTTAT |
| | | CTGCAAATGAACAGCCTCAG |
| | | AGCCGAGGACACGGCCGTGT |
| | | ATTACTGTGCGGGGGACACC |
| | | TTTGGAGTGGGACACTTCTA |
| | | CTGGGGCCAGGGAACCCTGG |
| | | TCACCGTCTCAAGC |
| 174R-E01 | 30 | GATGTTGTGATGACTCAGTC |
| VL | | TCCACTCTCCCTGCCCGTCA |
| (poly | | CCCCTGGAGAGCCGGCCTCC |
| nucleotide) | | ATCTCCTGCAGGTCTAGTCA |
| | | GAGCCTCCTGCATAGTAATG |
| | | GATACAACTATTTGGATTGG |
| | | TACCTGCAGAAGCCAGGGCA |
| | | GTCTCCACAGCTCCTGATCT |
| | | ATTTGGGTTCTAATCGGGCC |
| | | TCCGGGGTCCCTGACAGGTT |
| | | CAGTGGCAGTGGATCAGGCA |
| | | CAGACTTTACACTGCAAATC |
| | | AGCAGAGTGGAGGCTGAGGA |
| | | TGTTGGGGTCTATTACTGCA |
| | | TGCAAGGACTTCACACTCCG |
| | | GTCACTTTCGGCGGAGGGAC |
| | | CAAGGTGGAGATCAAA |
| 57A10 | 31 | CAGGTGCAGCTGGTGCAGTC |
| VH | | TGGGGCTGAGGTGAAGAAGC |
| (poly | | CTGGGTCCTCGGTGAAGGTC |
| nucleotide) | | TCCTGCAAGGCTTCTGGAGG |
| | | CACCTTCAGCAGCTCTGTTA |
| | | TCAGCTGGGTGCGGCAGGCC |
| | | CCTGGACAAGGGCTTGAGTG |
| | | GATGGGATGGATCAGTGTTT |
| | | ACAATGGTAACACAAACTAT |
| | | GCAGAGAAGTTCCAGGGCCG |
| | | GGTCACGATTACCGCGGACG |
| | | AATCCACGAGCACAGCCTAC |
| | | ATGGAGCTGAGCAGCCTGCG |
| | | GTCTGAGGACACGGCCGTGT |
| | | ATTACTGTGCGAGATTTGCT |
| | | ATGGTTCGGGGAGGGGTCTA |
| | | CTACTTTGACTACTGGGGCC |
| | | AGGGAACCCTGGTCACCGTC |
| | | TCCTCA |
| 57A10 | 32 | GACATCCAGATGACCCAGTC |
| VL | | TCCATCCTCCCTGTCTGCAT |
| (poly | | CTGTAGGAGACAGAGTCACC |
| nucleotide) | | ATCACTTGCCGGGCGAGTCA |
| | | GGGCATTAGCAGTTATTTAA |
| | | ATTGGTATCAGCAGAAACCA |
| | | GGGAAAGCCCCTAAGCTCCT |
| | | GATCTATGCTGCATCCAGTT |
| | | TGCAAAGTGGGGTCCCATCA |
| | | AGGTTCAGTGGCAGTGGATC |
| | | TGGGACAGATTTCACTCTCA |
| | | CCATCAGCAGTCTGCAACCT |
| | | GAAGATTTTGCAACTTACTA |
| | | CTGTCAACATTATTATAATC |
| | | TCCCGCTCACCTTCGGCCAA |
| | | GGTACCCGACTGGAGATTAA |
| | | A |
| 57SE8 | 33 | EVQLVQSGAEVKKPGASVKV |
| VH | | SCKASGYTFTNYYMHWVRQA |
| (poly | | PGQGLEWMGWINPNSGGTNY |
| peptide) | | AQKFQGRVTMTRDTSTSTVY |
| | | MELSSLRSEDTAVYYCARGH |
| | | WYFDLWGRGTLVTVSS |
| 57SE8 | 34 | DIQMTQSPSSLSASVGDRVT |
| VL | | ITCRASESIRSWLAWYQQKP |
| (poly | | GKAPKLLIYGASRLQSGVPS |
| peptide) | | RFSGSGSGTDFTLTISSLQP |
| | | EDFATYYCQQSYSTPWTFGQ |
| | | GTKVEIK |
| 57SB8 | 35 | EVQLVQSGAEVKKPGSSVKV |
| VH | | SCKASGYTFTKDYMHWVRQA |
| (poly | | PGQGLEWMGGIIPIFGTANY |
| peptide) | | AQRFQGRVTITADESTSTAY |
| | | MELSSLRSEDTAVYYCARGL |
| | | PPAAGGGGYFQHWGQGTLVT |
| | | VSS |
| 57SB8 | 36 | DIQMTQSPSSLSASVGDRVT |
| VL | | ITCRASQNVNDWLAWYQQKP |
| (poly | | GKAPKLLIYSASNLQSGVPS |
| peptide) | | RFSGSGSGTDFTLTIRSLQP |
| | | EDFATYYCQQSYSTPFTFGP |
| | | GTKVDIK |
| 174R-E01 | 37 | EVQLVQSGGGVVQPGRSLRL |
| VH | | SCAASGFTFSSYGMHWVRQA |
| (poly | | PGKGLEWVAVISYDGSNKYY |
| peptide) | | ADSVKGRFTISRDNSKNTLY |
| | | LQMNSLRAEDTAVYYCAGDT |
| | | FGVGHFYWGQGTLVTVSS |
| 174R-E01 | 38 | DVVMTQSPLSLPVTPGEPAS |
| VL | | ISCRSSQSLLHSNGYNYLDW |
| (poly | | YLQKPGQSPQLLIYLGSNRA |
| peptide) | | SGVPDRFSGSGSGTDFTLQI |
| | | SRVEAEDVGVYYCMQGLHTP |
| | | VTFGGGTKVEIK |
| 57A10 | 39 | EVQLVQSGAEVKKPGSSVKV |
| VH | | SCKASGGTFSSSVISWVRQA |
| (poly | | PGQGLEWMGWISVYNGNTNY |
| peptide) | | AEKFQGRVTITADESTSTAY |
| | | MELSSLRSEDTAVYYCARFA |
| | | MVRGGVYYFDYWGQGTLVTV |
| | | SS |
| 57A10 | 40 | DIQMTQSPSSLSASVGDRVT |
| VL | | ITCRASQGISSYLNWYQQKP |
| (poly | | GKAPKLLIYAASSLQSGVPS |
| peptide) | | RFSGSGSGTDFTLTISSLQP |
| | | EDFATYYCQHYYNLPLTFGQ |
| | | GTRLEIK |

In certain embodiments, the disclosure provides for polypeptides comprising a sequence having at least 80%, at least 85%, at least 90%, at least 95%, at least 98%, or at least 99% identity to any of SEQ ID NOs: 7-14 or 33-40. In certain embodiments, the disclosure provides for a WNT agonist comprising a FZD binding domain comprising a sequence having at least 80%, at least 85%, at least 90%, at least 95%, at least 98%, or at least 99% identity to any of SEQ ID NOs: 7-14 or 33-40. In certain embodiments, the disclosure provides for an antibody or antigen binding fragment thereof comprising a sequence having at least 80%, at least 85%, at least 90%, at least 95%, at least 98%, or at least 99% identity to any of SEQ ID NOs: 33-40. In certain embodiments, the disclosure provides for a combination molecule comprising a FZD binding domain comprising a sequence having at least 80%, at least 85%, at least 90%, at least 95%, at least 98%, or at least 99% identity to any of SEQ ID NOs: 7-14 or 33-40.

Table 6 provides the CDR sequences of the VH and VL of the above FZD5, 8 binding domains. In certain embodiments, the disclosure provides for polypeptides comprising either or both the VH and/or VL CDR sequences of any of the FZD5, 8 binding domains. In certain embodiments, the disclosure provides for a WNT agonist comprising a FZD binding domain comprising either or both the heavy chain (CDRH1-3) and/or light chain (CDRL1-3) CDR sequences of any of the FZD5, 8 binding domains identified herein, e.g., 57SE8, 57SB8174RE, or 57SA10. In certain embodiments, the disclosure provides for an antibody or antigen binding fragment thereof comprising either or both the VH and/or VL CDR sequences of any of the FZD5, 8 binding domains. In certain embodiments, the disclosure provides for a WNT agonist comprising either or both the VH and/or VL CDR sequences of any of the FZD5, 8 binding domains. In certain embodiments, the disclosure provides for a combination molecule comprising a FZD binding domain comprising either or both the VH and/or VL CDR sequences of any of the FZD5, 8 binding domains. In other embodiments, the polypeptide, antibody or binding fragment thereof, WNT agonist, or combination molecule comprises at least 5 of the six CDRs present in any of the binding domains. In other embodiments, the polypeptide, antibody or binding fragment thereof, WNT agonist, or combination molecule comprises the six CDRs present in any of the binding domains, wherein the CDRs collectively comprise one or more, e.g., one, two, three, four, five six or more amino acid modifications as compared to the native CDRs. In particular embodiments, a WNT agonist or combination molecule comprises two heavy chains and two lights chains collectively having any of the disclosed CDRs or variants thereof.

TABLE 6
CDR Sequences of FZD5, 8 binding domains

| Fzd5 | | | | | | |
|---|---|---|---|---|---|---|
| binders | CDRH1 | CDRH2 | CDRH3 | CDRL1 | CDRL2 | CDRL3 |
| 57SE8 | YTFTN | GWINP | CARGH | RASES | GASRL | CQQSY |
| | YYMH | NSGGT | WYFDL | IRSWL | QS | STPWT |
| | (SEQ | NYA | W | A | (SEQ | F |
| | ID | (SEQ | (SEQ | (SEQ | ID | (SEQ |
| | NO: | ID | ID | ID | NO: | ID |
| | 41) | NO: | NO: | NO: | 57) | NO: |
| | | 45) | 49) | 53) | | 61) |
| 57SB8 | YTFTK | GGIIP | CARGL | RASQN | SASNL | CQQSY |
| | DYMH | IFGTA | PPAAG | VNDWL | QS | STPFT |
| | (SEQ | NYA | GGGYF | A | (SEQ | F |
| | ID | (SEQ | QHW | (SEQ | ID | (SEQ |
| | NO: | ID | (SEQ | ID | NO: | ID |
| | 42) | NO: | ID | NO: | 58) | NO: |
| | | 46) | NO: | 54) | | 62) |
| | | | 50) | | | |
| 174RE01 | FTFSS | AVISY | CAGDT | RSSQS | LGSNR | CMQGL |
| | YGMH | DGSNK | FGVGH | LLHSN | AS | HTPVT |
| | (SEQ | YYA | FYW | GYNYL | (SEQ | F |
| | ID | (SEQ | (SEQ | D | ID | (SEQ |
| | NO: | ID | ID | (SEQ | NO: | ID |
| | 43) | NO: | NO: | ID | 59) | NO: |
| | | 47) | 51) | NO: | | 63) |
| | | | | 55) | | |
| 57SA10 | GTFSS | GWISV | CARFA | RASQG | AASSL | QHYYN |
| | SVIS | YNGNT | MVRGG | ISSYL | QS | LPLTF |
| | (SEQ | NYA | VYYFD | N | (SEQ | |
| | ID | (SEQ | YW | (SEQ | ID | (SEQ |
| | NO: | ID | (SEQ | ID | NO: | ID |
| | 44) | NO: | ID | NO: | 60) | NO: |
| | | 48) | NO: | 56) | | 64) |
| | | | 52) | | | |

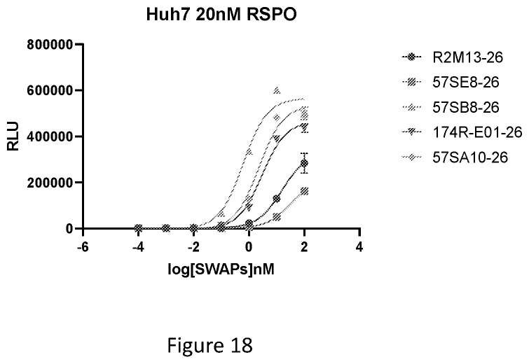

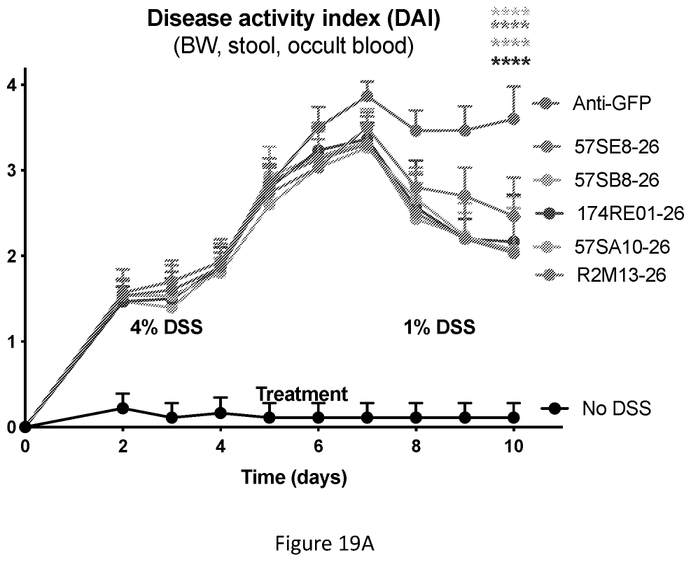

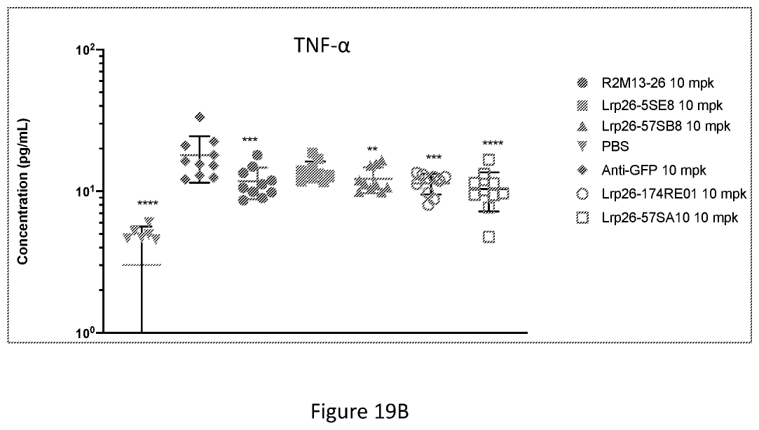

Cells were seeded at 1 million per 96-well plate on Day 1 and grown overnight. Proteins were added to the cells in 10 fold dilutions starting from 100 nM in triplicates in the presence of 20 nM R-spondin. Luciferase reporter activities were assayed in the wells with Luciferase Assay System (Promega) and read on a SpectraMax plate reader (Molecular Devices). Mean absolute RLU values of the triplicates for each protein dilution are shown (FIG. 18). R2M13-26 was included in the same assay for comparison.

To determine the efficacy of additional FZD5,8-specific WNT agonists in the DSS mouse model of IBD, six-week old C57Bl/6J female mice were obtained from Jackson Laboratories (Bar Harbor, Me., USA) and were housed 5 per cage. All animal experimentation was in accordance with the criteria of the “Guide for the Care and Use of Laboratory Animals” prepared by the National Academy of Sciences. Protocols for animal experimentation were approved by the Surrozen Institutional Animal Care and Use Committee.

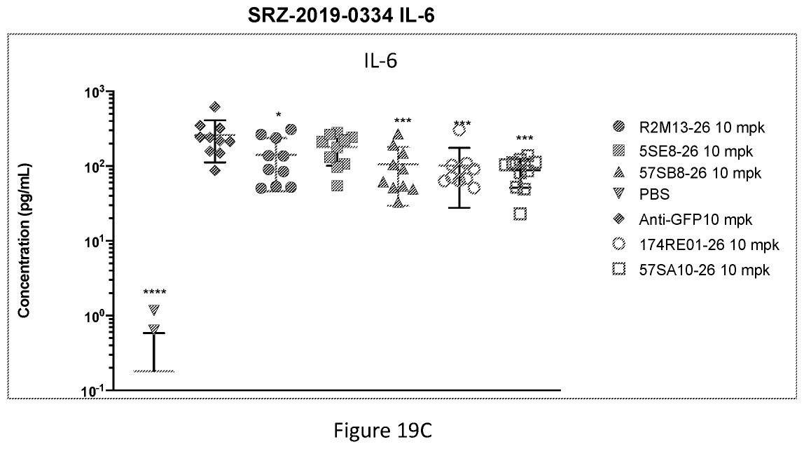

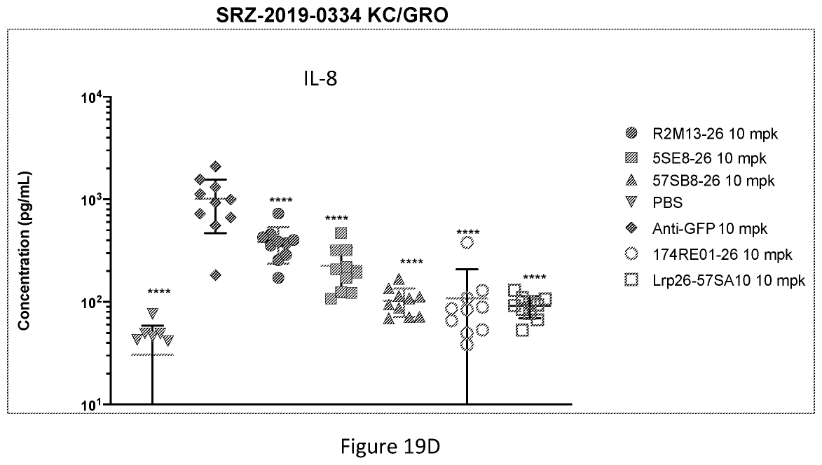

To induce acute colitis, 7- to 8-week-old female mice were given drinking water containing 4.0% (w/v) Dextran Sulfate Sodium (DSS, MP Biomedicals, MFCD00081551) ad libitum for 7 days and drinking water containing 1.0% (w/v) DSS for 3 days. Control mice subjected to DSS developed severe colitis characterized by profound and sustained weight loss and bloody diarrhea, resulting in the increase of disease activity index (DAI, FIG. 19A).

FZD5,8-specific WNT agonists treatment at 10 mpk, twice weekly, improved DAI, similar to R2M13-26 (FIG. 19A). The inflammatory cytokines in the serum were analyzed by Proinflammatory Panel 1 kits (Meso Scale Diagnostics, K15048D), and the results indicated that the FZD5,8-specific WNT agonists, except 58SE8-26, all significantly decreased TNF-α, IL-6 and IL-8 level in serum FIGS. 16A-16C) and in the colon tissue (FIG. 19B-19D).

FZD5,8-specific WNT agonists reduced intestinal inflammation and repaired epithelial damage leading to improved DAI in DSS colitis mouse model, further validating the FZD5, 8 specific WNT agonist molecules described herein, in treating acute colitis through improvement of the intestinal epithelial barrier.

### XI. Tissue Specific WNT Signal Enhancing Molecules Effectively Activated WNT Signaling and Stimulated Intestinal Organoid Growth In Vitro

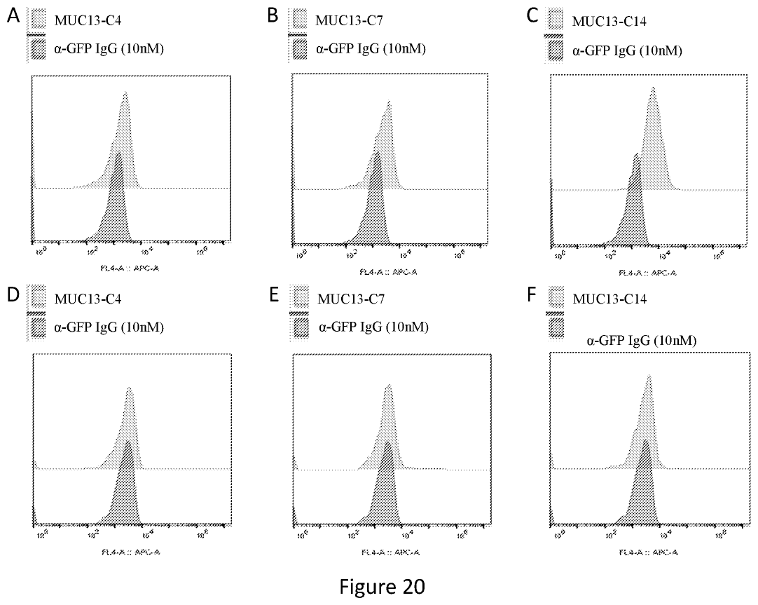

The MUC-13 binders C4, C7, and C14 (see, e.g., WO2016168607A1) were cloned and produced in the full-length IgG format and their binding capacity to MUC-13 was determined by FACS analysis to MUC-13 expressing HT29 cells. Their potential binding to MUC-13 non-expressing HEK293 cells was also analyzed as a negative control. Cells were harvested and washed 2× with FACS buffer (PBS (—Ca²⁺, —Mg²⁺), 0.1% BSA, 0.5% sodium Azide) and resuspended in FACS buffer at 10⁶ cells/ml. 60 μl of the cell suspension was aliquoted to each well of a 96-well v bottom plate, and the plate was spun for 3 min at 1500 rpm to remove the FACS buffer before adding corresponding MUC-13 antibody or anti-GFP control antibody at 10 nM diluted in FACS buffer and incubated at 4C for 1 hour. Plate was then spun to remove the primary antibodies and washed 1× with FACS buffer before adding Alexa Fluor® 488 goat anti-human secondary antibody (ThermoFisher Scientific) and incubating at 4C for 30 min. This media was then removed after spinning, and the plate washed 1× in FACS buffer. Cells were then resuspended in 150 FACS buffer and analyzed on a BD Accuri™ Cell Analyzers (Becton Dickinson) at 10,000 events. Comparing FACS plots for the HT29 (FIGS. 20A-20C) and for the HEK293 cells (FIGS. 20D-20F), only one of the MUC-13 binders tested, C14, showed specific FACS shift in HT29 cells, indicating MUC-13 specific binding activity of C14. Table 7 provides the sequences of the MUC-13 binders tested.

TABLE 7
Tissue Targeted WNT Enhancers

| | SEQ | |
|---|---|---|
| WNT | ID | |
| ENHANCER | NO: | SEQUENCE |
| C4-MUC-13 | 15 | DVQLQESGPGLVKPSQSLSL |
| HC | | TCSVTGYSITSGYYWNWIRQ |
| | | FPGNKLEWMGYISYDGSNNY |
| | | NPSLKNRISITRDTSKNQFF |
| | | LKLNSVTTEDTATYYCVRVP |
| | | TMITSYYFDYWGQGTTLTVS |
| | | SASTKGPSVFPLAPSSKSTS |
| | | GGTAALGCLVKDYFPEPVTV |
| | | SWNSGALTSGVHTFPAVLQS |
| | | SGLYSLSSVVTVPSSSLGTQ |
| | | TYICNVNHKPSNTKVDKKVE |
| | | PKSCDKTHTCPPCPAPEAAG |
| | | GPSVFLFPPKPKDTLMISRT |
| | | PEVTCVVVDVSHEDPEVKFN |
| | | WYVDGVEVHNAKTKPREEQY |
| | | NSTYRVVSVLTVLHQDWLNG |
| | | KEYKCKVSNKALGAPIEKTI |
| | | SKAKGQPREPQVYTLPPSRE |
| | | EMTKNQVSLTCLVKGFYPSD |
| | | IAVEWESNGQPENNYKTTPP |
| | | VLDSDGSFFLYSKLTVDKSR |
| | | WQQGNVFSCSVMHEALHNHY |
| | | TQKSLSLSPGK |
| C4-MUC-13 | 16 | QIVLTQSPAIMSASPGEKVT |
| LC | | ISCSASSSVGYIYWYQQKPG |
| | | SSPKPWIYRTSNLASGVPAR |
| | | FSGSGSGTSYSLTISSMEAE |
| | | DAATYYCQQYHSYPPTFGGG |
| | | TKLEIKRADRTVAAPSVFIF |
| | | PPSDEQLKSGTASVVCLLNN |
| | | FYPREAKVQWKVDNALQSGN |
| | | SQESVTEQDSKDSTYSLSST |
| | | LTLSKADYEKHKVYACEVTH |
| | | QGLSSPVTKSFNRGEC |
| C4-mutRSPO2 | 17 | DVQLQESGPGLVKPSQSLSL |
| HC | | TCSVTGYSITSGYYWNWIRQ |
| | | FPGNKLEWMGYISYDGSNNY |
| | | NPSLKNRISITRDTSKNQFF |
| | | LKLNSVTTEDTATYYCVRVP |
| | | TMITSYYFDYWGQGTTLTVS |
| | | SASTKGPSVFPLAPSSKSTS |
| | | GGTAALGCLVKDYFPEPVTV |
| | | SWNSGALTSGVHTFPAVLQS |
| | | SGLYSLSSVVTVPSSSLGTQ |
| | | TYICNVNHKPSNTKVDKKVE |
| | | PKSCDKTHTCPPCPAPEAAG |
| | | GPSVFLFPPKPKDTLMISRT |
| | | PEVTCVVVDVSHEDPEVKFN |
| | | WYVDGVEVHNAKTKPREEQY |
| | | NSTYRVVSVLTVLHQDWLNG |
| | | KEYKCKVSNKALGAPIEKTI |
| | | SKAKGQPREPQVYTLPPSRE |
| | | EMTKNQVSLTCLVKGFYPSD |
| | | IAVEWESNGQPENNYKTTPP |
| | | VLDSDGSFFLYSKLTVDKSR |
| | | WQQGNVFSCSVMHEALHNHY |
| | | TQKSLSLSPGKGGGGSGSGG |
| | | SGGGGSNPICKGCLSCSKDN |
| | | GCSRCQQKLFFFLRREGMRQ |
| | | YGECLHSCPSGYYGHRAPDM |
| | | NRCARCRIENCDSCRSKDAC |
| | | TKCKVGFYLHRGRCFDECPD |
| | | GFAPLEETMECVE |
| C7-MUC-13 | 18 | QVQLQQSGAELVRPGASVTL |
| HC | | SCKASGYTFHDYEIHWVKQT |
| | | PVYGLEWIGAIDPETGGTAY |
| | | NQKFKDKATLTADKSSSKAY |
| | | VEFRSLTSEDSAVYYCTIVR |
| | | GFWGQGTLVTVSAASTKGPS |
| | | VFPLAPSSKSTSGGTAALGC |
| | | LVKDYFPEPVTVSWNSGALT |
| | | SGVHTFPAVLQSSGLYSLSS |
| | | VVTVPSSSLGTQTYICNVNH |
| | | KPSNTKVDKKVEPKSCDKTH |
| | | TCPPCPAPEAAGGPSWLFPP |
| | | KPKDTLMISRTPEVTCVVVD |
| | | VSHEDPEVKFNWYVDGVEVH |
| | | NAKTKPREEQYNSTYRVVSV |
| | | LTVLHQDWLNGKEYKCKVSN |
| | | KALGAPIEKTISKAKGQPRE |
| | | PQVYTLPPSREEMTKNQVSL |
| | | TCLVKGFYPSDIAVEWESNG |
| | | QPENNYKTTPPVLDSDGSFF |
| | | LYSKLTVDKSRWQQGNVFSC |
| | | SVMHEALHNHYTQKSLSLSP |
| | | GK |
| C7-MUC-13 | 19 | DVLMTQTPLSLPVSLGDQAS |
| LC | | ISCRSGQTIVHSDGNIYLEW |
| | | YLQKPGQSPKLLIYKVSNRF |
| | | SGVPDRFSGSASGTDFTLKI |
| | | SRVEAEDLGVYYCFQGSHIP |
| | | FTFGGGTELEIKRADRTVAA |
| | | PSVFIFPPSDEQLKSGTASV |
| | | VCLLNNFYPREAKVQWKVDN |
| | | ALQSGNSQESVTEQDSKDST |
| | | YSLSSTLTLSKADYEKHKVY |
| | | ACEVTHQGLSSPVTKSFNRG |
| | | EC |
| C7-mutRSPO2 | 20 | QVQLQQSGAELVRPGASVTL |
| HC | | SCKASGYTFHDYEIHWVKQT |
| | | PVYGLEWIGAIDPETGGTAY |
| | | NOKFKDKATLTADKSSSKAY |
| | | VEFRSLTSEDSAVYYCTIVR |
| | | GFWGQGTLVTVSAASTKGPS |
| | | VFPLAPSSKSTSGGTAALGC |
| | | LVKDYFPEPVTVSWNSGALT |
| | | SGVHTFPAVLQSSGLYSLSS |
| | | VVTVPSSSLGTQTYICNVNH |
| | | KPSNTKVDKKVEPKSCDKTH |
| | | TCPPCPAPEAAGGPSVFLFP |
| | | PKPKDTLMISRTPEVTCVVV |
| | | DVSHEDPEVKFNWYVDGVEV |
| | | HNAKTKPREEQYNSTYRVVS |
| | | VLTVLHQDWLNGKEYKCKVS |
| | | NKALGAPIEKTISKAKGQPR |
| | | EPQVYTLPPSREEMTKNQVS |
| | | LTCLVKGFYPSDIAVEWESN |
| | | G |
| | | QPENNYKTTPPVLDSDGSFF |
| | | LYSKLTVDKSRWQQGNVFSC |
| | | SVMHEALHNHYTQKSLSLSP |
| | | GKGGGGSGSGGSGGGGSNPI |
| | | CKGCLSCSKDNGCSRCQQKL |
| | | FFFLRREGMRQYGECLHSCP |
| | | SGYYGHRAPDMNRCARCRIE |
| | | NCDSCRSKDACTKCKVGFYL |
| | | HRGRCFDECPDGFAPLEETM |
| | | ECVE |
| C14-MUC-13 | 21 | QVQLQQSGAELVRPGSSVKI |
| HC | | SCKASGYAFSTYWMNWVKQR |
| | | PGQGLEWIGQIYPGDGDTYY |
| | | NGNFKGKATLTADKSSSTAY |
| | | MQLSSLTSEDSAVYFCAVFW |
| | | DGYWGQGTTLTVSSASTKGP |
| | | SVFPLAPSSKSTSGGTAALG |
| | | CLVKDYFPEPVTVSWNSGAL |
| | | TSGVHTFPAVLQSSGLYSLS |
| | | SVVTVPSSSLGTQTYICNVN |
| | | HKPSNTKVDKKVEPKSCDKT |
| | | HTCPPCPAPEAAGGPSVFLF |
| | | PPKPKDTLMISRTPEVTCVV |
| | | VDVSHEDPEVKFNWYVDGVE |
| | | VHNAKTKPREEQYNSTYRVV |
| | | SVLTVLHQDWLNGKEYKCKV |
| | | SNKALGAPIEKTISKAKGOP |
| | | REPQVYTLPPSREEMTKNQV |
| | | SLTCLVKGFYPSDIAVEWES |
| | | NGQPENNYKTTPPVLDSDGS |
| | | FFLYSKLTVDKSRWQQGNVF |
| | | SCSVMHEALHNHYTQKSLSL |
| | | SPGK |
| C14-MUC-13 | 22 | QIVLTQSPTIMSASPGEKVT |
| LC | | MTCSASSSVTYIHWYQQKSG |
| | | TSPKRWIYDTSKLASGVPAR |
| | | FGGSGSGTSYSLTINSMETE |
| | | DAATYYCQQWSSNPFTFGSG |
| | | TKLEIKRADRTVAAPSVFIF |
| | | PPSDEQLKSGTASWCLLNNF |
| | | YPREAKVQWKVDNALQSGNS |
| | | QESVTEQDSKDSTYSLSSTL |
| | | TLSKADYEKHKVYACEVTHQ |
| | | GLSSPVTKSFNRGEC |
| C14-mutRSPO2 | 23 | QVQLQQSGAELVRPGSSVKI |
| HC | | SCKASGYAFSTYWMNVVVKQ |
| | | RPGQGLEWIGQIYPGDGDTY |
| | | YNGNFKGKATLTADKSSSTA |
| | | YMQLSSLTSEDSAVYFCAVF |
| | | WDGYWGQGTTLTVSSASTKG |
| | | PSVFPLAPSSKSTSGGTAAL |
| | | GCLVKDYFPEPVTVSWNSGA |
| | | LTSGVHTFPAVLQSSGLYSL |
| | | SSVVTVPSSSLGTQTYICNV |
| | | NHKPSNTKVDKKVEPKSCDK |
| | | THTCPPCPAPEAAGGPSVFL |
| | | FPPKPKDTLMISRTPEVTCV |
| | | VVDVSHEDPEVKFNWYVDGV |
| | | EVHNAKTKPREEQYNSTYRV |
| | | VSVLTVLHQDWLNGKEYKCK |
| | | VSNKALGAPIEKTISKAKGQ |
| | | PREPQVYTLPPSREEMTKNQ |
| | | VSLTCLVKGFYPSDIAVEWE |
| | | SNGQPENNYKTTPPVLDSDG |
| | | SFFLYSKLTVDKSRWQQGNV |
| | | FSCSVMHEALHNHYTQKSLS |
| | | LSPGKGGGGSGSGGSGGGGS |
| | | NPICKGCLSCSKDNGCSRCQ |
| | | QKLFFFLRREGMRQYGECLH |
| | | SCPSGYYGHRAPDMNRCARC |
| | | RIENCDSCRSKDACTKCKVG |
| | | FYLHRGRCFDECPDGFAPLE |
| | | ETMECVE |

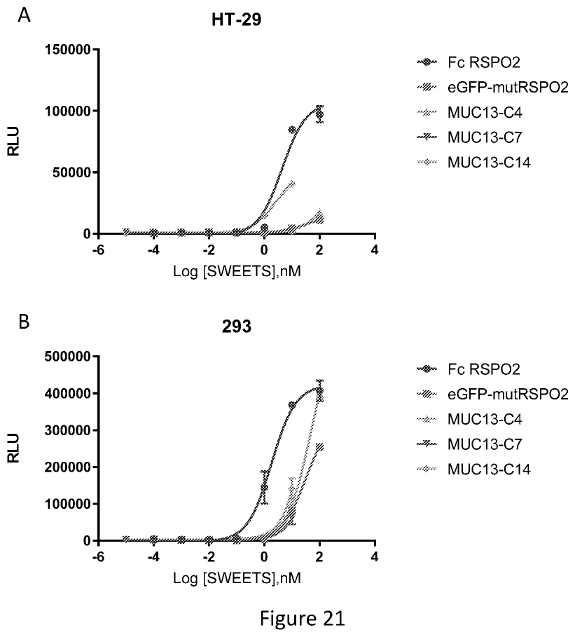

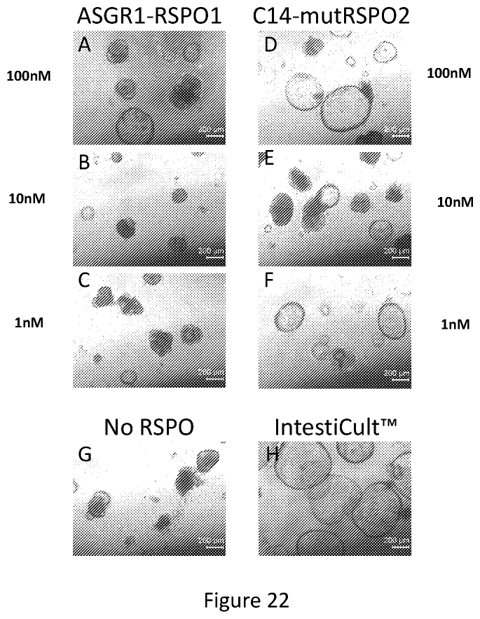

To examine whether the above noted MUC-13 binders can serve to drive tissue specificity for intestine specific WNT signaling enhancing molecules, the mutant (F105R/F109A) RSPO2 (mutRSPO2, which has the amino acid mutations in the Furin2 binding domain, thus reducing binding to LGR1-4 (see, e.g., WO2020014271)) was fused to the C-terminus of the heavy chain of the MUC-13 IgG antibodies (or GFP antibody as a negative control) with a 15 amino acid GS linker. The signaling activity of these of these MUC-13 targeted mutRSPO2 (mutRSPO) molecules was tested by Super TOPFlash luciferase reporter (STF) assay in HT29 cells or HEK293 cells, as described above. Dose response curves for C4-mutRSPO2, C7-mutRSPO2, and C14-mutRSPO2 luciferase reporter activities were measured (FIG. 21). Again, among the MUC-13 targeted WNT enhancing molecules, only C14-mutRSPO2 demonstrated a specific left shift of the dose response curve in HT29 cells but not in HEK293 cells, with an EC50 comparable to wildtype Fc-RSPO2 (SEQ ID NO:24). This is consistent with the MUC-13 binding activity of C-14 as IgG suggesting that when targeted by the MUC-13 binding, the WNT enhancing molecule which lacks Lgr4-6 binding capacity, can function like native RSPO2 to modulate WNT signaling in cells.

The signaling activities of the MUC-13 targeted WNT signaling enhancing molecules were also examined in human small intestine organoids. Growth and maintenance of organoid cultures was described above. Growth of human small intestine organoids was maintained when wildtype RSPO was replaced with C14-mutRSPO2 in the media. Human small intestine organoids were grown in basal media in which RSPO-1 was replaced by a non-intestine epithelial cell targeted mutRSPO1 (ASGR1-mutRSPO1, see, e.g., WO2020014271) at the concentration dilution series indicated (FIGS. 21A-21C) or by C14-mutRSPO2 at the same concentrations (FIGS. 21D-21F). While organoids grown in ASGR1-mutRSPO1 stopped growing and started to degenerate, similar to what observed when growing in basal media without any RSPO (FIG. 21G), C14-mutRSPO was able to maintain organoid growth similar to IntestiCult™ (StemCell Technologies) organoid growth media which contains wildtype RSPO (FIG. 21H). This assay demonstrated the MUC-13 targeted WNT signaling enhancing molecule can function on intact epithelium in human small intestine mini-tissue.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to

employ concepts of the various patents, applications and publications to provide yet further embodiments. These and other changes can be made to the embodiments in light of the above-detailed description.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method of treating a subject suffering from a gastrointestinal disorder comprising administering the subject an engineered WNT signaling modulator.

2. The method of claim 1, wherein the engineered WNT signaling modulator is an engineered WNT agonist.

3. The method of claim 1, wherein the engineered WNT signaling modulator is selected from the group consisting of an engineered polypeptide, an engineered antibody containing at least one epitope binding domain, a small molecule, an siRNA, and an antisense nucleic acid molecule.

4. The method of claim 2, wherein the engineered WNT agonist comprises one or more binding composition that binds to one or more FZD receptors (FZD1-10) and one or more binding composition that binds to one or more LRP receptors (LRP5-6).

5. The method of claim 4, wherein the binding compositions of the engineered WNT agonist comprise:
a) one or more binding composition that binds to:
i) FZD5;
ii) FZD 8;
iii) FZD 1;
iv) FZD 2;
vi) FZD 7;
vi) FZD 5 and FZD 8;
vii) FZD 1, FZD 2, and FZD 7;
viii) FZD 1, FZD 2, FZD 7, FZD 5 and FZD 8;
ix) FZD4;
x) FZD9; or
xi) FZD 10; and
b) one or more binding composition that binds to:
i) LRP5;
ii) LRP6; or
iii) LRP5/6.

6. The method of claim 5, wherein the WNT agonist comprises one or more binding composition that binds FZD5 and FZD8, and one or more binding composition that binds LRP5 or LRP6.

7. The method of claim 6, wherein the WNT agonist comprises a binding composition that binds to FZD5 and FZD8, and a binding composition that binds LRP6.

8. The method of claim 5, wherein the WNT agonist comprises a variable heavy chain sequence of SEQ ID NO: 1, 3, 5, 7, 9, 11, or 13; and a variable light chain sequence of SEQ ID NO: 2, 4, 6, 8, 10, 12, or 14.

9. The method of any of claims 1-8 wherein the engineered WNT signaling modulator repairs intestinal epithelium and/or reduces expression inflammatory cytokine.

10. The method of any of claims 1-8, wherein the engineered WNT signaling modulator comprises a tissue targeting molecule.

11. The method of claim 10, wherein the tissue targeting molecule is an antibody or fragment thereof that binds to a tissue specific cell surface antigen.

12. The method of claim 11, wherein the tissue targeting molecule is selected from the group consisting GPA33, CDH17, and MUC-13 polypeptides, and functional fragments or variants thereof.

13. The method of any of claims 1-8, wherein the WNT signaling modulator is administered with a binding composition that specifically binds an inflammatory molecule.

14. The method of claim 13, wherein the binding composition specifically binding the inflammatory molecule is an antagonist of the inflammatory molecule.

15. The method of claim 14, wherein the antagonist of the inflammatory molecule is an antagonist of TNFα, IL-12, IL-12 and IL-23, or IL-23.

16. The method of any of claims 1-8, wherein the gastrointestinal disease is inflammatory bowel disease.

17. The method of claim 16, wherein the inflammatory bowel disease is selected from the group consisting of: Crohn's disease (CD), CD with fistula formation, and ulcerative colitis (UC).

18. A method of treating a subject suffering from a gastrointestinal disorder comprising administering to the subject, a tissue-specific WNT signal enhancing molecule.

19. The method of claim 18, wherein the WNT signal enhancing molecule is an engineered molecule comprising:
a. a first domain that binds to one or more E3 ubiquitin ligases; and
b. a second domain that binds to a tissue specific receptor.

20. The method of claim 19, wherein the one or more E3 ubiquitin ligases are selected from the group consisting of: Zinc and Ring Finger Protein 3 (ZNRF3) and Ring Finger Protein 43 (RNF43).

21. The method of claim 19, wherein the first domain comprises an R-spondin (RSPO) polypeptide.

22. The method of claim 21, wherein the RSPO polypeptide is selected from the group consisting of RSPO-1, RSPO-2, RSPO-3, and RSPO-4.

23. The method of claim 21, wherein the RSPO polypeptide comprises a first furin domain and a second furin domain.

24. The method of claim 23, wherein the second furin domain is wild-type or is mutated to have lower binding to Leucine-rich repeat-containing G protein coupled receptors 4-6 (LGR4-6).

25. The method of claim 18, wherein the WNT signal enhancing molecule comprises a tissue targeting molecule.

26. The method of claim 25, wherein the tissue targeting molecule is an antibody or fragment thereof that binds to a tissue specific cell surface antigen.

27. The method of claim 26, wherein the tissue targeting molecule is selected from the group consisting of GPA33, CDH17, and MUC-13 polypeptides, and functional fragments and variants thereof.

28. The method of claim 27, wherein the WNT signal enhancing molecule comprises a heavy chain sequence of SEQ ID NO: 17, 20, or 23; and a light chain sequence of SEQ ID NO: 16, 19, or 22.

29. The method of any of claims 18-28, wherein the WNT signal enhancing molecule is administered with a binding composition that specifically binds an inflammatory molecule.

30. The method of claim 29, wherein the binding composition that specifically binds the inflammatory molecule is an antagonist of the inflammatory molecule.

31. The method of claim 30, wherein the antagonist of the inflammatory molecule is an antagonist of TNFα, IL-12, IL-12 and IL-23, or IL-23.

32. The method of any of claims 18-28, wherein the gastrointestinal disease is inflammatory bowel disease.

33. The method of claim 32, wherein the inflammatory bowel disease is selected from the group consisting of: Crohn's disease (CD), CD with fistula formation, and ulcerative colitis (UC).

34. A method of treating a subject suffering from a gastrointestinal disorder comprising administering to the subject an engineered WNT agonist and an engineered tissue specific WNT signal enhancing combination molecule.

35. The method of claim 34, wherein the combination molecule comprises:
a) the engineered WNT agonist selected from the group consisting of a FZD 5 binding composition, a FZD 8 binding composition, a FZD 1 binding composition, a FZD 2 binding composition, a FZD 7 binding composition, a LRP5 binding composition, a LRP6 binding composition, and a LRP5/6 binding composition; and
b) the engineered WNT signal enhancing molecule comprising a first domain that binds to one or more E3 ubiquitin ligases; and a second domain that binds to a tissue specific receptor.

36. The method of claim 35, wherein the E3 ubiquitin ligases are selected from the group consisting of Zinc and Ring Finger Protein 3 (ZNRF3) and Ring Finger Protein 43 (RNF43).

37. The method of claim 35, wherein the first domain comprises an R-spondin (RSPO) polypeptide.

38. The method of claim 37, wherein the RSPO polypeptide is selected from the group consisting of RSPO-1, RSPO-2, RSPO-3, and RSPO-4.

39. The method of claim 37, wherein the RSPO polypeptide comprises a first furin domain and a second furin domain.

40. The method of claim 39, wherein the second furin domain is wild-type or is mutated to have lower binding to Leucine-rich repeat-containing G protein coupled receptors 4-6 (LGR4-6).

41. The method of claim 34, wherein the combination molecule incorporates a tissue targeting molecule.

42. The method of claim 41, wherein the tissue targeting molecule is an antibody or fragment thereof that binds to a tissue specific cell surface antigen.

43. The method of claim 42, wherein the tissue targeting molecule is selected from the group consisting of GPA33, CDH17, and MUC-13 polypeptides, and functional fragments and variants thereof.

44. The method of any of claims 34-42, wherein the combination molecule is administered with a binding composition that specifically binds an inflammatory molecule.

45. The method of claim 44, wherein the binding composition specific for the inflammatory molecule is an antagonist of the inflammatory molecule.

46. The method of claim 45, wherein the antagonist of the inflammatory molecule is an antagonist of TNFα, IL-12, IL-12 and IL-23, or IL-23.

47. The method of any of claims 34-42, wherein the gastrointestinal disease is inflammatory bowel disease.

48. The method of claim 47, wherein the inflammatory bowel disease is selected from the group consisting of: Crohn's disease (CD), CD with fistula formation, and ulcerative colitis (UC).

49. A polypeptide that specifically binds Frizzed 5 (FZD5) and Frizzled 8 (FZD8), wherein the polypeptide comprises one or more sequence having at least 80%, at least 90%, or at least 95% homology to a sequence set forth in any of SEQ ID NOs: 33-40 or encoded by any of SEQ ID NOs: 33-40.

50. The polypeptide of claim 49, wherein said polypeptide comprises an antibody or antibody binding fragment.

51. The polypeptide of claim 50, wherein said antibody or antibody binding fragment comprises at least 5 or all six of the CDRs present in any of the following combinations of sequence: SEQ ID NOs:33 and 34; SEQ ID NOs:35 and 36; SEQ ID NOs:37 and 38; or SEQ ID NOs:39 and 40.

52. The polypeptide of claim 50, wherein said polypeptide comprises six of the CDRs present in any of the of the following combinations of sequence: SEQ ID NOs:33 and 34; SEQ ID NOs:35 and 36; SEQ ID NOs:37 and 38; or SEQ ID NOs:39 and 40, wherein one or more of the CDRs comprises one, two, or three amino acid modifications, optionally a point mutation, an amino acid deletion, or an amino acid insertion.

53. An engineered WNT agonist comprising:
(a) one or more binding domains that bind to FZD5 and FZD8, wherein at least one of the one or more binding domains comprises a polypeptide of any one of claims 49-52; and
(b) one or more binding domains that bind to LRP5, LRP6, or both LRP5 and LRP6.

54. An engineered WNT agonist comprising a polypeptide sequence having at least 80%, at least 90%, or at least 95% homology to any one of SEQ ID NOs: 7-14.

55. The engineered WNT agonist of claim 54, comprising:
(a) a polypeptide sequence having at least 80%, at least 90%, or at least 95% homology to SEQ ID NO: 7 and a polypeptide sequence having at least 80%, at least 90%, or at least 95% homology to SEQ ID NO:8;
(b) a polypeptide sequence having at least 80%, at least 90%, or at least 95% homology to SEQ ID NO: 9 and a polypeptide sequence having at least 80%, at least 90%, or at least 95% homology to SEQ ID NO:10;
(c) a polypeptide sequence having at least 80%, at least 90%, or at least 95% homology to SEQ ID NO: 11 and a polypeptide sequence having at least 80%, at least 90%, or at least 95% homology to SEQ ID NO:12; or
(d) a polypeptide sequence having at least 80%, at least 90%, or at least 95% homology to SEQ ID NO: 13 and a polypeptide sequence having at least 80%, at least 90%, or at least 95% homology to SEQ ID NO:14.

56. A combination molecule comprising:
a) the engineered WNT agonist of any one of claims 53-55; and
b) an engineered WNT signal enhancing molecule comprising a first domain that binds to one or more E3 ubiquitin ligases; and a second domain that binds to a tissue specific receptor.

57. A pharmaceutical composition comprising the polypeptide of any one of claims 49-52, the engineered WNT agonist of any one of claims 53-55, or the combination molecule of claim 56.

58. A method of treating a subject suffering from a gastrointestinal disorder comprising administering to the subject the engineered WNT agonist of any one of claims 53-55, the combination molecule of claim 56, or the pharmaceutical composition of claim 57.

59. The method of claim 58, wherein the gastrointestinal disorder is an inflammatory bowel disease, optionally selected from the group consisting of: Crohn's disease (CD), CD with fistula formation, and ulcerative colitis (UC).


SEQUENCE LISTING (64 sequences)

SEQ ID NO: 1 - protein, 452 aa, Artificial Sequence
  Made in Lab - synthesized wnt agonist
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCASSKEKATYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 340 aa, Artificial Sequence
  Made in Lab - synthesized wnt agonist
DVQLVESGGGLVQAGGSLRLACAGSGRIFAIYDIAWYRHPPGNQRELVAMIRPVVTEIDYADSVKGRFTISRNNAMKTVYLQMNNLKPEDTAVYYCNAKRPWGSRDEYWGQGTQVTVSSGSGSGQAVVLQEPSLSVSPGGTVTLTCGLSSGSVSTNYYPSWYQQTPGQAPRTLIYYTNTRSSDVPERFSGSIVGNKAALTITGAQPDDESVYFCLLYLGRGIWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 452 aa, Artificial Sequence
  Made in Lab - synthesized wnt agonist
EVQLLQSGAEVKKPGSSVKVSCKASGGTFTYRYLHWVRQAPGQGLEWMGGIIPIFGTGNYAQKFQGRVTITADESTSTAYMELSSLRSEDTAVYYCASSMVRVPYYYGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 339 aa, Artificial Sequence
  Made in Lab - synthesized wnt agonist
DVQLVESGGGLVQPGGSLRLSCTSSANINSIETLGWYRQAPGKQRELIANMRGGGYMKYAGSLKGRFTMSTESAKNTMYLQMNSLKPEDTAVYYCYVKLRDDDYVYRGQGTQVTVSSGGSGSGSGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 454 aa, Artificial Sequence
  Made in Lab - synthesized wnt agonist
QVQLQQWGAGLLKPSETLSLTCAVSGASFSGHYWTWIRQPPGKGLEWIGEIDHTGSTNYEPSLRSRVTISVDTSKNQFSLNLKSVTAADTAVYYCARGGQGGYDWGHYHGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 335 aa, Artificial Sequence
  Made in Lab - synthesized wnt agonist
DVQLVESGGGLVQPGGSLRLSCTSSANINSIETLGWYRQAPGKQRELIANMRGGGYMKYAGSLKGRFTMSTESAKNTMYLQMNSLKPEDTAVYYCYVKLRDDDYVYRGQGTQVTVSSGSGSGSYVLTQPPSVSVSPGQTASITCSGDKVGHKYASWYQQKPGQSPVLVIYEDSQRPSGIPVRFSGSNSGNTATLTISGTQAMDEADYYCQAWDSSTDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 446 aa, Artificial Sequence
  Made in Lab - synthesized wnt agonist
EVQLVQSGAEVKKPGASVKVSCKASGYTFTNYYMHWVRQAPGQGLEWMGWINPNSGGTNYAQKFQGRVTMTRDTSTSTVYMELSSLRSEDTAVYYCARGHWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 338 aa, Artificial Sequence
  Made in Lab - synthesized wnt agonist
DVQLVESGGGLVQAGGSLRLACAGSGRIFAIYDIAWYRHPPGNQRELVAMIRPVVTEIDYADSVKGRFTISRNNAMKTVYLQMNNLKPEDTAVYYCNAKRPWGSRDEYWGQGTQVTVSSGGGGSDIQMTQSPSSLSASVGDRVTITCRASESIRSWLAWYQQKPGKAPKLLIYGASRLQSGVPSRFSGSGSGTDFTLTISSLQPEDFATYYCQQSYSTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 453 aa, Artificial Sequence
  Made in Lab - synthesized wnt agonist
EVQLVQSGAEVKKPGSSVKVSCKASGYTFTKDYMHWVRQAPGQGLEWMGGIIPIFGTANYAQRFQGRVTITADESTSTAYMELSSLRSEDTAVYYCARGLPPAAGGGGYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 338 aa, Artificial Sequence
  Made in Lab - synthesized wnt agonist
DVQLVESGGGLVQAGGSLRLACAGSGRIFAIYDIAWYRHPPGNQRELVAMIRPVVTEIDYADSVKGRFTISRNNAMKTVYLQMNNLKPEDTAVYYCNAKRPWGSRDEYWGQGTQVTVSSGGGGSDIQMTQSPSSLSASVGDRVTITCRASQNVNDWLAWYQQKPGKAPKLLIYSASNLQSGVPSRFSGSGSGTDFTLTIRSLQPEDFATYYCQQSYSTPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 448 aa, Artificial Sequence
  Made in Lab - synthesized wnt agonist
EVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAGDTFGVGHFYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 343 aa, Artificial Sequence
  Made in Lab - synthesized wnt agonist
DVQLVESGGGLVQAGGSLRLACAGSGRIFAIYDIAWYRHPPGNQRELVAMIRPVVTEIDYADSVKGRFTISRNNAMKTVYLQMNNLKPEDTAVYYCNAKRPWGSRDEYWGQGTQVTVSSGGSGSDVVMTQSPLSLPVTPGEPASISCRSSQSLLHSNGYNYLDWYLQKPGQSPQLLIYLGSNRASGVPDRFSGSGSGTDFTLQISRVEAEDVGVYYCMQGLHTPVTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 452 aa, Artificial Sequence
  Made in Lab - synthesized wnt agonist
EVQLVQSGAEVKKPGSSVKVSCKASGGTFSSSVISWVRQAPGQGLEWMGWISVYNGNTNYAEKFQGRVTITADESTSTAYMELSSLRSEDTAVYYCARFAMVRGGVYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 338 aa, Artificial Sequence
  Made in Lab - synthesized wnt agonist
DVQLVESGGGLVQAGGSLRLACAGSGRIFAIYDIAWYRHPPGNQRELVAMIRPVVTEIDYADSVKGRFTISRNNAMKTVYLQMNNLKPEDTAVYYCNAKRPWGSRDEYWGQGTQVTVSSGGGGSDIQMTQSPSSLSASVGDRVTITCRASQGISSYLNWYQQKPGKAPKLLIYAASSLQSGVPSRFSGSGSGTDFTLTISSLQPEDFATYYCQHYYNLPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 451 aa, Artificial Sequence
  Made in Lab - synthesized wnt enhancer
DVQLQESGPGLVKPSQSLSLTCSVTGYSITSGYYWNWIRQFPGNKLEWMGYISYDGSNNYNPSLKNRISITRDTSKNQFFLKLNSVTTEDTATYYCVRVPTMITSYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 216 aa, Artificial Sequence
  Made in Lab - synthesized wnt enhancer
QIVLTQSPAIMSASPGEKVTISCSASSSVGYIYWYQQKPGSSPKPWIYRTSNLASGVPARFSGSGSGTSYSLTISSMEAEDAATYYCQQYHSYPPTFGGGTKLEIKRADRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 573 aa, Artificial Sequence
  Made in Lab - synthesized wnt enhancer
DVQLQESGPGLVKPSQSLSLTCSVTGYSITSGYYWNWIRQFPGNKLEWMGYISYDGSNNYNPSLKNRISITRDTSKNQFFLKLNSVTTEDTATYYCVRVPTMITSYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGSGGSGGGGSNPICKGCLSCSKDNGCSRCQQKLFFFLRREGMRQYGECLHSCPSGYYGHRAPDMNRCARCRIENCDSCRSKDACTKCKVGFYLHRGRCFDECPDGFAPLEETMECVE

SEQ ID NO: 18 - protein, 443 aa, Artificial Sequence
  Made in Lab - synthesized wnt enhancer
QVQLQQSGAELVRPGASVTLSCKASGYTFHDYEIHWVKQTPVYGLEWIGAIDPETGGTAYNQKFKDKATLTADKSSSKAYVEFRSLTSEDSAVYYCTIVRGFWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 222 aa, Artificial Sequence
  Made in Lab - synthesized wnt enhancer
DVLMTQTPLSLPVSLGDQASISCRSGQTIVHSDGNIYLEWYLQKPGQSPKLLIYKVSNRFSGVPDRFSGSASGTDFTLKISRVEAEDLGVYYCFQGSHIPFTFGGGTELEIKRADRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 565 aa, Artificial Sequence
  Made in Lab - synthesized wnt enhancer
QVQLQQSGAELVRPGASVTLSCKASGYTFHDYEIHWVKQTPVYGLEWIGAIDPETGGTAYNQKFKDKATLTADKSSSKAYVEFRSLTSEDSAVYYCTIVRGFWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGSGGSGGGGSNPICKGCLSCSKDNGCSRCQQKLFFFLRREGMRQYGECLHSCPSGYYGHRAPDMNRCARCRIENCDSCRSKDACTKCKVGFYLHRGRCFDECPDGFAPLEETMECVE

SEQ ID NO: 21 - protein, 444 aa, Artificial Sequence
  Made in Lab - synthesized wnt enhancer
QVQLQQSGAELVRPGSSVKISCKASGYAFSTYWMNWVKQRPGQGLEWIGQIYPGDGDTYYNGNFKGKATLTADKSSSTAYMQLSSLTSEDSAVYFCAVFWDGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 216 aa, Artificial Sequence
  Made in Lab - synthesized wnt enhancer
QIVLTQSPTIMSASPGEKVTMTCSASSSVTYIHWYQQKSGTSPKRWIYDTSKLASGVPARFGGSGSGTSYSLTINSMETEDAATYYCQQWSSNPFTFGSGTKLEIKRADRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 566 aa, Artificial Sequence
  Made in Lab - synthesized wnt enhancer
QVQLQQSGAELVRPGSSVKISCKASGYAFSTYWMNWVKQRPGQGLEWIGQIYPGDGDTYYNGNFKGKATLTADKSSSTAYMQLSSLTSEDSAVYFCAVFWDGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGSGGSGGGGSNPICKGCLSCSKDNGCSRCQQKLFFFLRREGMRQYGECLHSCPSGYYGHRAPDMNRCARCRIENCDSCRSKDACTKCKVGFYLHRGRCFDECPDGFAPLEETMECVE

SEQ ID NO: 24 - protein, 349 aa, Artificial Sequence
  Made in Lab - synthesized wnt enhancer
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVE

SEQ ID NO: 25 - DNA, 348 bp, Artificial Sequence
  Synthesized polynucleotide
  misc_feature (48)..(48): n is a, c, g, or t
  misc_feature (63)..(63): n is a, c, g, or t
  misc_feature (84)..(84): n is a, c, g, or t
caggtgcagctggtgcagtctggggctgaggtgaagaagcctggggcntcagtgaaggtttcntgcaaggcatctggatacacnttcaccaactactatatgcactgggtgcgtcaggcccctggacaagggcttgagtggatgggatggatcaaccctaacagtggtggcacaaattatgcacagaagtttcagggccgtgtcaccatgacccgcgacacgtccacgagcacagtctacatggagctgagcagcctgcgttctgaggacacggccgtgtattactgtgcgagagggcactggtacttcgatctctggggccgtggcaccctggtcaccgtctcctca

SEQ ID NO: 26 - DNA, 321 bp, Artificial Sequence
  Synthesized polynucleotide
gacatccggatgacccagtctccatcctccctgtctgcatctgtaggagacagagtcaccatcacttgccgggccagtgagagtattaggagctggttggcctggtatcagcagaaaccagggaaagcccctaagctcctgatctatggtgcatcgcgtttgcaaagtggggtcccatcaaggttcagtggcagtggatctgggacagatttcactctcaccatcagcagtctgcaacctgaagattttgcaacttactactgtcaacagagttacagtaccccttggacgttcggccaaggtaccaaggtggaaatcaaa

SEQ ID NO: 27 - DNA, 369 bp, Artificial Sequence
  Synthesized polynucleotide
gaggtgcagctggtgcagtctggggctgaggtgaagaagcctgggtcctcggtgaaggtctcctgcaaggcttctggatacaccttcaccaaagactatatgcactgggtgcggcaggcccctggacaagggcttgagtggatgggagggatcatccctatatttggtacagcaaactacgcacagaggttccagggccgggtcacgattaccgcggacgaatccacgagcacagcctacatggagctgagcagcctgcggtctgaggacacggccgtgtattactgtgcgagaggactcccaccagcagctggtggcggcggatacttccagcactggggccagggcaccctggtcaccgtctcctca

SEQ ID NO: 28 - DNA, 321 bp, Artificial Sequence
  Synthesized polynucleotide
gacatccagatgacccagtctccatcctccctgtctgcatctgtaggagacagagtcaccatcacttgccgggccagtcagaatgttaatgactggttggcctggtatcagcagaaaccagggaaagcccctaagctcctgatctatagtgcatccaatttgcaatctggggtcccatcaaggttcagtggcagtggatctgggacagatttcactctcaccatccgcagtctgcaacctgaagattttgcaacttactactgtcaacagagctacagtaccccattcactttcggccctggtaccaaagtggatatcaaa

SEQ ID NO: 29 - DNA, 354 bp, Artificial Sequence
  Synthesized polynucleotide
gaggtccagctggtgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctttatctgcaaatgaacagcctcagagccgaggacacggccgtgtattactgtgcgggggacacctttggagtgggacacttctactggggccagggaaccctggtcaccgtctcaagc

SEQ ID NO: 30 - DNA, 336 bp, Artificial Sequence
  Synthesized polynucleotide
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagactttacactgcaaatcagcagagtggaggctgaggatgttggggtctattactgcatgcaaggacttcacactccggtcactttcggcggagggaccaaggtggagatcaaa

SEQ ID NO: 31 - DNA, 366 bp, Artificial Sequence
  Synthesized polynucleotide
caggtgcagctggtgcagtctggggctgaggtgaagaagcctgggtcctcggtgaaggtctcctgcaaggcttctggaggcaccttcagcagctctgttatcagctgggtgcggcaggcccctggacaagggcttgagtggatgggatggatcagtgtttacaatggtaacacaaactatgcagagaagttccagggccgggtcacgattaccgcggacgaatccacgagcacagcctacatggagctgagcagcctgcggtctgaggacacggccgtgtattactgtgcgagatttgctatggttcggggaggggtctactactttgactactggggccagggaaccctggtcaccgtctcctca

SEQ ID NO: 32 - DNA, 321 bp, Artificial Sequence
  Synthesized polynucleotide
gacatccagatgacccagtctccatcctccctgtctgcatctgtaggagacagagtcaccatcacttgccgggcgagtcagggcattagcagttatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacattattataatctcccgctcaccttcggccaaggtacccgactggagattaaa

SEQ ID NO: 33 - protein, 116 aa, Artificial Sequence
  Synthesized polypeptide
EVQLVQSGAEVKKPGASVKVSCKASGYTFTNYYMHWVRQAPGQGLEWMGWINPNSGGTNYAQKFQGRVTMTRDTSTSTVYMELSSLRSEDTAVYYCARGHWYFDLWGRGTLVTVSS

SEQ ID NO: 34 - protein, 107 aa, Artificial Sequence
  Synthesized polypeptide
DIQMTQSPSSLSASVGDRVTITCRASESIRSWLAWYQQKPGKAPKLLIYGASRLQSGVPSRFSGSGSGTDFTLTISSLQPEDFATYYCQQSYSTPWTFGQGTKVEIK

SEQ ID NO: 35 - protein, 123 aa, Artificial Sequence
  Synthesized polypeptide
EVQLVQSGAEVKKPGSSVKVSCKASGYTFTKDYMHWVRQAPGQGLEWMGGIIPIFGTANYAQRFQGRVTITADESTSTAYMELSSLRSEDTAVYYCARGLPPAAGGGGYFQHWGQGTLVTVSS

SEQ ID NO: 36 - protein, 107 aa, Artificial Sequence
  Synthesized polypeptide
DIQMTQSPSSLSASVGDRVTITCRASQNVNDWLAWYQQKPGKAPKLLIYSASNLQSGVPSRFSGSGSGTDFTLTIRSLQPEDFATYYCQQSYSTPFTFGPGTKVDIK

SEQ ID NO: 37 - protein, 118 aa, Artificial Sequence
  Synthesized polypeptide
EVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAGDTFGVGHFYWGQGTLVTVSS

SEQ ID NO: 38 - protein, 112 aa, Artificial Sequence
  Synthesized polypeptide
DVVMTQSPLSLPVTPGEPASISCRSSQSLLHSNGYNYLDWYLQKPGQSPQLLIYLGSNRASGVPDRFSGSGSGTDFTLQISRVEAEDVGVYYCMQGLHTPVTFGGGTKVEIK

SEQ ID NO: 39 - protein, 122 aa, Artificial Sequence
  Synthesized polypeptide
EVQLVQSGAEVKKPGSSVKVSCKASGGTFSSSVISWVRQAPGQGLEWMGWISVYNGNTNYAEKFQGRVTITADESTSTAYMELSSLRSEDTAVYYCARFAMVRGGVYYFDYWGQGTLVTVSS

SEQ ID NO: 40 - protein, 107 aa, Artificial Sequence
  Synthesized polypeptide
DIQMTQSPSSLSASVGDRVTITCRASQGISSYLNWYQQKPGKAPKLLIYAASSLQSGVPSRFSGSGSGTDFTLTISSLQPEDFATYYCQHYYNLPLTFGQGTRLEIK

SEQ ID NO: 41 - protein, 9 aa, Artificial Sequence
  Synthesized polypeptide - CDRH1
YTFTNYYMH

SEQ ID NO: 42 - protein, 9 aa, Artificial Sequence
  Synthesized polypeptide - CDRH1
YTFTKDYMH

SEQ ID NO: 43 - protein, 9 aa, Artificial Sequence
  Synthesized polypeptide - CDRH1
FTFSSYGMH

SEQ ID NO: 44 - protein, 9 aa, Artificial Sequence
  Synthesized polypeptide - CDRH1
GTFSSSVIS

SEQ ID NO: 45 - protein, 13 aa, Artificial Sequence
  Synthesized polypeptide - CDRH2
GWINPNSGGTNYA

SEQ ID NO: 46 - protein, 13 aa, Artificial Sequence
  Synthesized polypeptide - CDRH2
GGIIPIFGTANYA

SEQ ID NO: 47 - protein, 13 aa, Artificial Sequence
  Synthesized polypeptide - CDRH2
AVISYDGSNKYYA

SEQ ID NO: 48 - protein, 13 aa, Artificial Sequence
  Synthesized polypeptide - CDRH2
GWISVYNGNTNYA

SEQ ID NO: 49 - protein, 11 aa, Artificial Sequence [sequence omitted]

SEQ ID NO: 50 - protein, 18 aa, Artificial Sequence
  Synthesized polypeptide - CDRH3
CARGLPPAAGGGGYFQHW

SEQ ID NO: 51 - protein, 13 aa, Artificial Sequence
  Synthesized polypeptide - CDRH3
CAGDTFGVGHFYW

SEQ ID NO: 52 - protein, 17 aa, Artificial Sequence
  Synthesized polypeptide - CDRH3
CARFAMVRGGVYYFDYW

SEQ ID NO: 53 - protein, 11 aa, Artificial Sequence
  Synthesized polypeptide - CDRL1
RASESIRSWLA

SEQ ID NO: 54 - protein, 11 aa, Artificial Sequence
  Synthesized polypeptide - CDRL1
RASQNVNDWLA

SEQ ID NO: 55 - protein, 16 aa, Artificial Sequence
  Synthesized polypeptide - CDRL1
RSSQSLLHSNGYNYLD

SEQ ID NO: 56 - protein, 11 aa, Artificial Sequence
  Synthesized polypeptide - CDRL1
RASQGISSYLN

SEQ ID NO: 57 - protein, 7 aa, Artificial Sequence
  Synthesized polypeptide - CDRL2
GASRLQS

SEQ ID NO: 58 - protein, 7 aa, Artificial Sequence
  Synthesized polypeptide - CDRL2
SASNLQS

SEQ ID NO: 59 - protein, 7 aa, Artificial Sequence
  Synthesized polypeptide - CDRL2
LGSNRAS

SEQ ID NO: 60 - protein, 7 aa, Artificial Sequence
  Synthesized polypeptide - CDRL2
AASSLQS

SEQ ID NO: 61 - protein, 11 aa, Artificial Sequence [sequence omitted]

SEQ ID NO: 62 - protein, 11 aa, Artificial Sequence [sequence omitted]

SEQ ID NO: 63 - protein, 11 aa, Artificial Sequence [sequence omitted]

SEQ ID NO: 64 - protein, 10 aa, Artificial Sequence
  Synthesized polypeptide - CDRL3
QHYYNLPLTF